(19) United States
(12) Patent Application Publication
(10) Pub. No.: US 2019/0167732 A1
(43) Pub. Date: Jun. 6, 2019

(54) THERAPEUTIC USES OF MICROVESICLES AND RELATED MICRORNAS

(71) Applicant: The University Court of the University of Glasgow, Glasgow (GB)

(72) Inventors: Paul Shiels, Glasgow (GB); Wayne Davies, Glasgow (GB)

(21) Appl. No.: 16/034,059

(22) Filed: Jul. 12, 2018

Related U.S. Application Data
(63) Continuation of application No. 15/829,160, filed on Dec. 1, 2017.
Application No. 15/829,160 is a continuation of application No. 15/487,223, filed on Apr. 13, 2017, now abandoned.
Application No. 15/487,223 is a continuation of application No. 14/987,931, filed on Jan. 5, 2016, now abandoned.
Application No. 14/987,931 is a continuation of application No. 13/766,666, filed on Feb. 13, 2013, now abandoned.
Application No. 13/766,666 is a continuation of application No. PCT/IB2011/002028, filed on Aug. 12, 2011.
(60) Provisional application No. 61/380,766, filed on Sep. 8, 2010.
(60) Provisional application No. 61/373,715, filed on Aug. 13, 2010.

(51) Int. Cl. A61K 35/407; A61K 35/39; C12N 15/88; G01N 33/50; A61K 35/28; A61K 9/50; A61K 35/14; C12N 15/113; A61K 35/22

(52) U.S. Cl. CPC A61K 35/407; A61K 35/39; C12N 15/88; G01N 33/5061; G01N 33/507; G01N 33/5073; A61K 2035/124; A61K 9/5068; A61K 35/14; C12N 15/113; A61K 35/22; C12N 2320/11; A61K 35/28

(57) ABSTRACT

The present invention provides improved methods and compositions based on microvesicles for the treatment of various diseases, disorders and conditions. In particular, the present invention encompasses the recognition that microvesicles contain specific microRNAs which may function as intercellular regulators involved in cell or tissue regeneration, remodeling, reconstruction, reprogramming or transdifferentiation. Thus, among other things, the present invention provides methods and compositions based on microvesicles and/or associated microRNAs that provide more predictable and effective therapeutic results.

### CROSS REFERENCE TO RELATED APPLICATIONS

This patent application is a continuation of international application No. PCT/IB2011/002028 filed on Aug. 12, 2011, which claims the benefit of U.S. Provisional Patent Application Ser. Nos. 61/373,715, filed Aug. 13, 2010 and 61/380,766, filed Sep. 8, 2010, the entirety of each of which is incorporated herein by reference.

This application relates to international application PCT/IB2011/002048, and entitled “Cellular and Molecular Therapies” filed on Aug. 12, 2011, the entirety of which is incorporated herein by reference.

### SEQUENCE LISTING

The present specification makes reference to a Sequence Listing (submitted electronically as a .txt file named “Sequence Listing.txt on Feb. 13, 2013). The .txt file was generated on Feb. 13, 2013 and is 93.1 kb in size. The entire contents of the Sequence Listing are herein incorporated by reference.

### BACKGROUND

Microvesicles were historically regarded as cellular debris with no apparent function. However, and more recently, a growing body of experimental data suggest that microvesicles have numerous biological activities. For example, platelet-derived microvesicles were shown to stimulate selected cells via surface proteins on the microvesicles e.g., CD154, RANTES, and/or PF-4; see Thromb. Haemost. (1999), 82:794, or J. Biol. Chem. (1999), 274:7545). In other examples, specific effects of bioactive lipids (e.g., sphingosine-1-phosphate, HETE, or arachidonic acid) in platelet microvesicles on certain target cells were reported (see e.g., J. Biol. Chem. (2001), 276: 19672; or Cardiovasc. Res. (2001), 49(5):88). Furthermore, platelet microvesicles increased adhesion of mobilized CD34+endothelial cells by transfer of certain microvesicle surface components to the mobilized cells (see e.g., Blood (2001), 89:3143).

Various clinical uses of microvesicles have been proposed. While such proposed uses provide at least some promising perspectives, several largely unexplained problems remain. For example, biological activity of microvesicles is often difficult to predict. Moreover, currently contemplated therapeutic use typically necessitates sterilization and antiviral treatment to prevent infections of the people receiving microvesicle containing preparations, which is time-consuming and inefficient. Therefore, there is still a need for improved compositions and methods of use based on microvesicles.

### SUMMARY OF THE INVENTION

The present invention provides improved methods and compositions based on microvesicles for the treatment of various diseases, disorders and conditions. In particular, the present invention encompasses the recognition that microvesicles contain specific microRNAs which may function as intercellular regulators involved in cell or tissue regeneration, remodeling, reconstruction, reprogramming or transdifferentiation. Thus, the present invention provides methods and compositions based on microvesicles and/or associated microRNAs that provide more predictable and effective therapeutic results.

In some embodiments, the present invention provides a method of treating a disease, disorder or condition comprising administering to a patient in need of treatment a therapeutically effective amount of microvesicles. In some embodiments, inventive methods according to the present invention can be used to treat a disease, disorder or condition selected from the group consisting of diabetes mellitus, myocardial infarct, kidney disease, wound healing, Fistulas regeneration, neural regeneration (e.g., CNS regeneration, or peripheral nervous system regeneration), breast augmentation following mastectomy, conditions associated with a cosmetic surgical procedure, and combination thereof.

In some embodiments, the present invention provides a method of inducing tissue repair, remodeling, differentiation or transdifferentiation in vivo comprising administering to a patient in need of treatment a therapeutically effective amount of microvesicles. In some embodiments, suitable microvesicles are derived from a tissue that is the same as the diseased tissue (i.e., target tissue). In some embodiments, suitable microvesicles are derived from a tissue that is different from the diseased tissue(i.e., target tissue). In some embodiments, suitable microvesicles are derived from pancreatic cells, kidney cells, liver cells, spleen cells, lymph nodes, myometrium cells, peripheral blood cells, chord blood cells, bone marrow cells, serum, or combination thereof. In some embodiments, suitable microvesicles are derived from pancreas-derived pathfinder cells. In some embodiments, suitable microvesicles are derived from autologous cells. In some embodiments, suitable microvesicles are derived from non-autologous cells.

In some embodiments, suitable microvesicles are derived from cells grown on a nonwoven substrate. In some embodiments, the nonwoven substrate comprise an aliphatic polyester fiber. In some embodiments, a aliphatic polyester fiber suitable for the present invention is selected from the group consisting of homopolymers or copolymers of lactide (which includes lactic acid and meso lactide), glycolide (including glycolic acid), epsilon-caprolactone, p-dioxanone (1,4-dioxan-2-one), trimethylene carbonate (1,3-dioxan-2-one), and combinations thereof.

In some embodiments, suitable microvesicles are derived from cells grown under a culture condition where oxygen pressure is less than or equal to 5%. In some embodiments, suitable microvesicles are derived from cells grown under room air oxygen conditions. In some embodiments, suitable microvesicles are derived from cells grown to approximately 80-99% of confluence.

In some embodiments, suitable microvesicles are derived from cells grown under serum starvation conditions. In some embodiments, suitable microvesicles are derived from cells grown under serum starvation conditions for about 24 hours. In some embodiments, suitable microvesicles are derived from cells grown under serum replete conditions.

In some embodiments, suitable microvesicles are isolated or purified by differential ultracentrifugation. In some embodiments, suitable microvesicles are isolated or purified by precipitation.

In some embodiments, suitable microvesicles contain one or more microRNAs selected from those listed in Table 1 and Tables 7-13.

In some embodiments, suitable microvesicles contains one or more microRNAs selected form the group consisting of miRNA-122, miRNA-127, miRNA-133b, miRNA-323, miRNA-433, miRNA-451, miRNA-466h, miRNA-467c, miRNA-467e, miRNA-468, miRNA-491, miRNA-495, miRNA-546, miRNA-666, miRNA-680, miRNA-346, miRNA-136, miRNA-202, miRNA-369, miRNA-370, miRNA-375, miRNA-376b, miRNA-38I, miRNA-434, miRNA-452, miRNA-465a, miRNA-465b, miRNA-470, miRNA-487b, miRNA-543, miRNA-547, miRNA-590, miRNA-741, miRNA-881, miRNA-206, miRNA-224, miRNA-327, miRNA-347, and combination thereof.

In some embodiments, suitable microvesicles contain one or more microRNAs selected form the group consisting of miRNA-122, miRNA-127, miRNA-133b, miRNA-323, miRNA-433, miRNA-451, miRNA-466h, miRNA-467c, miRNA-467e, miRNA-468, miRNA-491, miRNA-495, miRNA-546, miRNA-666, miRNA-680, miRNA-346, and combination thereof.

In some embodiments, suitable microvesicles do not contain miRNA-129-5p, miRNA-190, miRNA-203, miRNA-32, miRNA-34c, miRNA-376c, miRNA-384-3p, miRNA-499b, miRNA-455, miRNA-582-5p, miRNA-615-3p, miRNA-615-5p, miRNA-7b, miRNA-17-3p, miRNA-381, and miRNA-505.

In some embodiments, a therapeutically effective amount of microvesicles ranges from 1 fg-1 mg/kg body weight (e.g., 10 fg-1 mg/kg, 100 fg-1 mg/kg, 1 pg-1 mg/kg, 10 pg-1 mg/kg, 100 pg-1 mg/kg body weight). In some embodiments, the microvesicles are administered intravenously, intra-arterially, intramuscularly, subcutaneously, cutaneously, intradermally, intracranially, intratheccally, intrapleurally, intra-orbitalty, intra nasally, orally, intra alimentrally, colorectally, and/or intra-cerebrospinally.

In some embodiments, the microvesicles are administered daily. In some embodiments, the microvesicles are administered weekly. In some embodiments, the microvesicles are administered biweekly. In some embodiments, the microvesicles are administered monthly.

In some embodiments, the present invention provides a method of treating a disease, disorder or condition by administering one or more microRNAs obtained, isolated or purified from microvesicles. In some embodiments, the microvesicles are derived from cells grown under serum starvation conditions. In some embodiments, the microvesicles are derived from cells grown under serum starvation conditions for about 24 hours. In some embodiments, the microvesicles are derived from cells grown under serum replete conditions. In some embodiments, the microRNAs obtained, isolated or purified from microvesicles are differentially expressed in cells and/or microvesicles derived from cells grown under stress conditions (e.g., oxygen pressure, cell culture confluence, serum amounts in medium, etc.). In some embodiments, the present invention provides a method of treating a disease, disorder or condition comprising administering to a patient in need of treatment a therapeutically effective amount of one or more microRNAs having a sequence at least 70% (e.g., 75%, 80%, 85%, 90%, 95%, 98%, 99%) identical to any of SEQ ID NOs:1-72 (e.g., SEQ ID NOs:1-29). In some embodiments, the one or more microRNAs have a sequence identical to any of SEQ ID NO:1-72 (e.g., SEQ ID NOs:1-29). In some embodiments, the present invention provides a method of treating a disease, disorder or condition comprising administering to a patient in need of treatment a therapeutically effective amount of one or more microRNAs having a sequence at least 70% (e. 75%, 80%, 85%, 90%, 95%, 98%, 99%) identical to any of the sequences in Tables 7-13.

In some embodiments, the present invention provides a method of inducing tissue repair, remodeling, differentiation or transdiferentiation in vivo comprising administering to a patient in need of treatment a therapeutically effective amount of one or more microRNAs having a sequence at least 70% (e.g., 75%, 80%, 85%, 90%, 95%, 98%, 99%) identical to any one of SEQ ID NO:1-72 (e.g., SEQ ID INOs:1-29). In some embodiments, the one or more microRNAs have a sequence identical to any of SEQ ID NO:1-72 (e.g., SEQ ID NOs: 1-29). In some embodiments, the present invention provides a method of inducing tissue repair, remodeling, differentiation or transdiferentiation in vivo comprising administering to a patient in need of treatment a therapeutically effective amount of one or more microRNAs having a sequence at least 70% (e.g., 75%, 80%, 85%, 90%, 95%, 98%, 99%) identical to any of the sequences in Tables 7-13.

In some embodiments, inventive methods according to the present invention can be used to treat a disease, disorder or condition selected from the group consisting of diabetes mellitus, myocardial infarct, kidney disease, wound healing, Fistulas regeneration, neural regeneration (e.g., CNS regeneration, or peripheral nervous system regeneration), breast augmentation following mastectomy, conditions associated with a cosmetic surgical procedure, and combination thereof.

In some embodiments, the therapeutically effective amount of the one or more miRNAs ranges from 1 fg-1 mg/kg body weight (e.g., 10 fg-1 mg/kg, 100 fg-1 mg/kg, 1 pg-1 mg/kg, 10 pg-1 mg/kg, 100 pg-1 mg/kg body weight). In some embodiments, the one or more miRNAs are administered intravenously, intra-arterially, intramuscularly, subcutaneously, cutaneously, intradermally, intracranially, intratheccally, intrapleurally, intra-orbitally, intra nasally, orally, intra alimentrally, colorectally, and/or intra-cerebrospinally. In some embodiments, the one or more miRNAs are administered intravenously, intra-arterially, intramuscularly, subcutaneously, cutaneously, intradermally, intracranially, intratheccally, intrapleurally, intra-orbitally, intra nasally, orally, intra alimentrally, colorectally, and/or intra-cerebrospinally. In some embodiments, the one or more miRNAs are administered daily, weekly, biweekly, or monthly.

In some embodiments, the present invention provides a pharmaceutical composition comprising a therapeutically effective amount of microvesicles for the treatment of various diseases, disorders or conditions. In some embodiments, the present invention provides a pharmaceutical composition comprising a therapeutically effective amount of microvesicles for the treatment of diabetes mellitus, myocardial infarct, kidney disease, wound healing, Fistulas regeneration, neural regeneration (e.g., CNS regeneration, or peripheral nervous system regeneration), breast augmentation following mastectomy, conditions associated with a cosmetic surgical procedure, and combination thereof.

In some embodiments, the present invention provides a pharmaceutical composition comprising one or more microRNAs having a sequence at least 70% (e.g., 75%, 80%, 85%, 90%, 95%, 98%, 99%) identical to any one of SEQ ID NO:1-72 (e.g., SEQ ID NOs:1-29) and a pharmaceutically acceptable carrier. In some embodiments, the present invention provides a pharmaceutical composition comprising one or more microRNAs having a sequence identical to any one of SEQ ID NO:1-72 (e.g., SEQ ID NOs:1-29) and a pharmaceutically acceptable carrier. In some embodiments, the present invention provides a pharmaceutical composition comprising one or more microRNAs having a sequence at least 70% (e.g., 75%, 80%, 85%, 90%, 95%, 98%, 99%) identical to any of the sequences in Tables 7-13 and a pharmaceutically acceptable carrier. In some embodiments, the present invention provides a pharmaceutical composition comprising one or more microRNAs having a sequence identical to any of the sequences in Tables 7-13 and a pharmaceutically acceptable carrier. In some embodiments, the one or more miRNAs are present in a therapeutically effective amount for the treatment of diabetes mellitus, myocardial infarct, kidney disease, wound healing, Fistulas regeneration, neural regeneration (e.g., CNS regeneration, or peripheral nervous system regeneration), breast augmentation following mastectomy, conditions associated with a cosmetic surgical procedure, or combination thereof.

In some embodiments, the present invention provides a method for identifying a miRNA that induces cell growth and/or regeneration, comprising providing cells grown in a microvesicle-depleted medium; adding an miRNA to the medium; determining if the addition of the miRNA increases cell proliferation rate as compared to a control, thereby identifying if the miRNA induces cell growth and/or regeneration. In sonic embodiments, the cells are pancreas-derived pathfinder cells. In some embodiments, the cell proliferation rate is determined by doubling time. In some embodiments, the miRNA is isolated from microvesicles.

In some embodiments, the present invention provides a method for identifying a miRNA that induces cell growth and/or regeneration, comprising creating a wounded area in cells grown to confluence; treating the cells with an miRNA; determining a rate of re-growth of the treated cells across the wounded area as compared to a control, thereby identifying if the miRNA induces cell growth and/or regeneration. In some embodiments, the cells are fibroblasts or cardiomyocytes. In some embodiments, the rate of re-growth is determined quantitatively,

In some embodiments, the control is untreated cells but otherwise grown under identical conditions. In some embodiments, the miRNA is isolated from microvesicles.

In some embodiments, the present invention provides an miRNA that induces cell growth and/or regeneration identified using a method described herein.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow, It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIGS. 1A and 1B depict exemplary scanning electron microscopy pictures of sub-confluent rat PDPCs adapted for growth in medium with fetal bovine serum (FBS) depleted for bovine microvesicles. Nascent microvesicles can be seen at the surfaces of cells in both figures.

FIGS. 2A and 2B show exemplary effects of MVs on growth rates of rat PDPCs. FIG. 2A depicts the effect of bovine MV depletion on doubling time of rat PDPCs. (Plotted on the y-axis is electrical impedence; negative values indicate cell death and therefore negative growth.) MV depletion was performed at 43 hours. A negative effect on doubling time was seen, with a later recovery. FIG. 2B depicts dose-dependent recovery of rat PDPC doubling time after addition of rat PDPC-derived MVs. Cultures were MV-depleted at 48 hours, and then exogenous MVs were added 10 hours later. The rapid recovery of doubling time of cells receiving exogenous MV occurred well in advance of the normal recovery time.

FIG. 3 depicts an exemplary differential centrifugation fractionation of microvesicle-containing cell culture medium.

FIG. 4 shows an exemplary diagram comparing miRNA expression profiles for rat PCs, MV fractions, and exosome fractions. The diagram shows the number of miRNAs whose expression is altered by growth under serum starvation conditions for 24 hours as compared with growth under serum replete conditions. Total rat miRNA genes analyzed =584. Total human miRNA genes analyzed =761. Data presented is from an N=1 experiment with a single gene expression analysis on the TLDA card.

FIG. 5 shows an exemplary graph comparison of miRNA expression profiled for rat PCs, MV fractions, and exosome fractions. The graph shows miRNAs with increased gene expression following growth under serum starvation conditions for 24 hours as compared with growth under serum replete conditions. Total rat miRNA genes analyzed =584. Data presented is from an N=1 experiment with a single gene expression analysis on the TLDA card.

FIG. 6 shows an exemplary diagram comparing miRNA expression profiles for rat PCs, rat MSC, and human PC. The chart shows the number of miRNAs whose expression is altered by growth under serum starvation conditions for 24 hours as compared with growth under serum replete conditions. Total rat miRNA genes analyzed =584. Total human miRNA genes analyzed =761. Data presented is from an N=1 experiment with a single gene expression analysis on the TLDA card.

FIG. 7 shows an exemplary diagram comparing miRNA expression profiles for human PCs and microvesicles (MVs) obtained from human PCs. The chart shows the number of miRNAs whose expression is altered by growth under serum starvation conditions for 24 hours as compared with growth under serum replete conditions. Total human miRNA genes analyzed =761. Data presented is from an N=1 experiment with a single gene expression analysis on the TLDA card.

FIG. 8 shows an exemplary diagram comparing miRNA expression profiles for MVs obtained from rat PCs and MVs obtained from human PCs. The diagram shows the number of miRNAs whose expression is altered by growth under serum starvation conditions for 24 hours as compared with growth under serum replete conditions. Total rat and mouse miRNA genes analyzed =584, Total human miRNA genes analyzed =761. Data presented is from an N=1 experiment with a single gene expression analysis on the TLDA card.

FIG. 9 shows an exemplary graph comparison of miRNA expression profile for MVs obtained from rat PCs and MVs obtained from human PCs. The graph shows miRNAs with increased or decreased gene expression following growth under serum starvation conditions for 24 hours as compared with growth under serum replete conditions. Total rat and mouse miRNA genes analyzed =584. Data presented is from an N=1 experiment with a single gene expression analysis on the TLDA card.

### DEFINITIONS

In order for the present invention to be more readily understood, certain terms are first defined below. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 1.9%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Autoimmune disorder: As used herein, the term “autoimmune disorder” refers to a disorder resulting from attack of a body's own tissue by its immune system. In some embodiments, autoimmune diseases is diabetes mellitus, multiple sclerosis, premature ovarian failure, scleroderma, Sjogren's disease, lupus, alopecia (baldness), polyglandular failure, Grave's disease, hypothyroidism, polymyosititis, Celiac disease, Crohn's disease, inflammatory bowel disease, ulcerative colitis, autoimmune hepatitis, hypopituitaristn, Guillain-Barre syndrome, myocardititis, Addison's disease, a.utoimmune skin diseases (e.g., psoriasis), uveititis, pernicious anemia, polymyalgia rheumatica, Goodpasture's syndrome, hypoparathyroidism, Hashimoto's thyoriditis, Ra.ynaud's phenomenon, potymyagli a rheumatica, and rheumatoid arthritis.

Autologous and non-autologus: As used herein, the term “autologous” means from the same organism. In the context of the present application, the term is used to mean that the population of cells and/or microvesicles referred to as “autologous” to each other do not contain any material which could be regarded as allogenic or xenogenic, that is to say derived from a “foreign” cellular source. As used herein, the term “non-autologous” means not from the same organism.

Diabetes mellitus: As used herein, the term “diabetes mellitus” refers to a metabolic disease characterized by abnormally high levels of glucose in the blood, caused by an inherited inability to produce insulin (Type 1) or an acquired resistance to insulin (Type 2). Type 1 diabetes is a severe, chronic form of diabetes caused by insufficient production of insulin and resulting in abnormal metabolism of carbohydrates, fats, and proteins. The disease, which typically appears in childhood or adolescence, is characterized by increased sugar levels in the blood and urine, excessive thirst, frequent urination, acidosis, and wasting. Type 1 diabetes is also called insulin-dependent diabetes. Type 2 diabetes is a mild form of diabetes that typically appears first in adulthood and is exacerbated by obesity and an inactive lifestyle. This disease often has no symptoms, is usually diagnosed by tests that indicate glucose intolerance, and is treated with changes in diet and an exercise regimen. Type 2 diabetes is also called non-insulin-dependent diabetes.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control. In some embodiments, a control is also referred to as a reference,

Cosmetic surgical procedure: As used herein, the term “cosmetic surgical procedure” refers to a procedure that is not directed to the therapy of a disease but is, rather, directed to the improvement of an individual's aesthetic appearance, particularly the appearance of the skin or hair of an individual. Examples of cosmetic surgical procedures include procedures that result in reduction in skin wrinkles, an increase in skin firmness, an increase in hair growth or shine, a reduction in grey hairs, a regrowth of hair in cases of baldness (especially male pattern baldness), reduction in hair growth (especially facial hair growth), an aesthetic enhancement of breast size or shape, and a reduction in cellulite.

Crude: As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

Derivative thereof: As used herein, the term “derivative thereof,” when used in connection with microvesicles or cells, refers to a fraction or extract (especially those containing RNA and/or DNA and/or protein) of the original microvesicle or population of cells which retains at least some biological activity (especially the ability to induce differentiation and/or the ability to provide therapeutic benefit) of the original. The term also include complexed, encapsulated or formulated microvesicles or cells (for example, microvesicles that have been encapsulated, complexed or formulated to facilitate administration). Examples of derivatives include lysates, lyophilates and homogenates.

Dysfunction: As used herein, the term “dysfunction” refers to an abnormal function. Dysfunction of a molecule (e.g., a protein) can be caused by an increase or decrease of an activity associated with such molecule. Dysfunction of a molecule can be caused by defects associated with the molecule itself or other molecules that directly or indirectly interact with or regulate the molecule.

Functional: As used herein, a “functional” biological molecule is a biological molecule in a form in which it exhibits a property and/or activity by which it is characterized.

Functional derivative: As used herein, the term “functional derivative” denotes, in the context of a functional derivative of a nucleotide sequence (e.g., microRNA), a molecule that retains a biological activity (either function or structural) that is substantially similar to that of the original sequence. A functional derivative or equivalent may be a natural derivative or is prepared synthetically. Exemplary functional derivatives include nucleotide sequences having substitutions, deletions, or additions of one or more nucleotides, provided that the biological activity of the nucleic acids (e.g., microRNAs) is conserved.

Inflammation: As used herein, the term “inflammation” includes inflammatory conditions occurring in many disorders which include, but are not limited to: Systemic Inflammatory Response (SIRS); Alzheimer's Disease (and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyper responsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma tymphomatosa (Hashimoto's thyroiditis, lymphadenoid goiter), sleep disorders and chronic fatigue syndrome and obesity (non-diabetic or associated with diabetes), Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

Inducer: As used herein, the term “inducer” refers to any molecule or other substance capable of inducing a change in the fate of differentiation of a cell to which it is applied.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal,

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man, Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

microRNA: As used herein, the term “microRNAs (miRNA.$)” refers to post-transcriptional regulators that typically bind to complementary sequences in the three prime untranslated regions (3′ UTRs) of target messenger RNA transcripts (mRNAs), usually resulting in gene silencing. Typically, tniRNAs are short ribonucleic acid (RNA) molecules, for example, 21 or 22 nucleotides long. The terms “microRNA” and “miRNA” are used interchangeably.

Microvesicle: As used herein, the term “microvesicle” refers to a membranaceus particle comprising fragments of plasma membrane derived from various cell types. Typically, microvesicles have a diameter (or largest dimension where the particle is not spheroid) of between about 10 nm to about 5000 nm (e.g., between about 50 nm and 1500 nm, between about 75 nm and 1500 nm, between about 75 nrn and 1250 nm, between about 50 nrn and 1250 nm, between about 30 nm and 1000 nm, between about 50 nm and 1000 nm, between about 100 nm and 1000 nm, between about 50 nm and 750 nm, etc.). Typically, at least part of the membrane of the microvesicle is directly obtained from a cell (also known as a donor cell). Microvesicles suitable for use in the present invention may originate from cells by membrane inversion, exocytosis, shedding, blebbing, and/or budding. Depending on the manner of generation (e.g., membrane inversion, exocytosis, shedding, or budding), the microvesicles contemplated herein may exhibit different surface/lipid characteristics. Alternative names for microvesicles include, but are not limited to, exosomes, ectosomses, membrane particles, exosome-like particles, and apoptotic vesicles. As used herein, an abbreviated form “MV” is sometime used to refer to microvesicle.

Pail finder cells: As used herein, the term “pathfinder cells” refers to cells that have the capacity to induce or stimulate tissue repair, regeneration, remodeling or differentiation. Typically, pathfinder cells induce or stimulate tissue repair, regeneration, remodeling or differentiation without being a source of new tissue themselves. In some embodiments, pathfinder cells are also referred to as “progenitor cells.” As used herein, an abbreviated form “PC” is sometime used to refer to pathfinder cell.

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition,

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition,

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent of the invention refers to a peptide inhibitor or derivatives thereof according to the invention.

Transdifferentiation: As used herein, the term “transdifferentiation” refers to a process in which a non-stem cell transforms into a different type of cell, or an already differentiated stem cell creates cells outside its already established differentiation path. Typically, transdifferentiation include de- and then re-differentiation of adult cell types (or differentiated cell types).

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Detailed Description of Certain Embodiments

The present invention provides, among other things, improved compositions and methods based on microvesicles or microvesicles-associated microRNAs for inducing tissue repair, remodeling, reconstruction, differentiation or transdifferentiation, and/or for treating associated diseases, disorders and conditions.

Various aspects of the invention are described in detail in the following sections. The use of sections is not meant to limit the invention. Each section can apply to any aspect of the invention. In this application, the use of “or” means “and/or” unless stated otherwise.

### I. Microvesicles

As used herein, the term “microvesicle” refers to a membranaceus particle comprising fragments of plasma membrane derived from various cell types. Typically, microvesicles are small particles that have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the microvesicle is directly obtained from a cell (also known as a donor cell). Microvesicles suitable for use in the present invention may originate from cells by membrane inversion, exocytosis, shedding, blebbing, and/or budding. Depending on the manner of generation (e.g., membrane inversion, exocytosis, shedding, or budding), the microvesicles contemplated herein may exhibit different surface/lipid characteristics. Alternative names for microvesicles include, but are not limited to, exosomes, ectosomses, membrane particles, exosome-like particles, and apoptotic vesicles.

It is contemplated that microvesicles can serve as a means by which RNA and protein molecules can pass between cells. Without wishing to be bound by any particular theory, it is contemplated that microvesicles derived from pancreas-derived Pathfinder cells (PDPCs) may stimulate repair processes through the transfer of specific mRNAs, miRNAs, and/or proteins. Prior to the present invention, however, the specific microRNAs associated with microvesicles have not yet been characterized. As discussed in the microRNA and the Examples sections, the present inventors have developed an effective in vitro assay to analyze and identify microRNAs. Unexpectedly, the inventors found that certain microRNAs are specifically present in microvesicles (i.e., present only in microvesicles and not cells). This finding demonstrated for the first time that microvesicles do not just contain randomly sampled cytoplasmic or endosomal contents. It is contemplated that those microRNAs that are specifically present in the microvesicles may be intracelullar regulators important for inducing tissue repair, remodeling, reconstruction, differentiation or transdifferentiation.

Donor Cells

Microvesicles used in accordance with the present invention may be obtained from any cell types. As used herein, cells that produce microvesicles are also referred to as donor cells. Suitable donor cells may include prokaryotic cells, archaebacterial cells, fungal cells, and single- and multi-cellular eukaryotic cells. In some embodiments, microvesicles are obtained from a eukaryotic cell (e.g., a eukaryotic cell from a multi-cellular organism, and particularly, a vertebrate cell (e.g., mammal)). Furthermore, it should be recognized that the donor cell may be nucleated or non-nucleated. Thus, suitable donor cells include lymphocytes (e.g., polynucleated, polymorpho-nuclear lymphocytes, etc), fibroblasts, hepatocytes, as well as erythrocytes, and thrombocytes.

Suitable donor cells may be derived from any desirable developmental stage with respect to its cell lineage. For example, suitable donor cells may include stem cells (which may or may not be committed to a particular cell line), partially differentiated stem cell, and fully differentiated cells. In some embodiments, suitable donor cells may be human embryonic stem cell-derived mesenchymal stem cells. In some embodiments, suitable donor cells are pathfinder cells. As used herein, the term “pathfinder cells” encompasses pluripotent cells that have the capacity to induce or stimulate tissue repair, regeneration, remodeling or differentiation. Pathfinder cells may be obtained from any of a variety of tissue types, including, but not limited to, pancreas, kidney, lymph node, liver, spleen, myometrium, blood cells (including cells from peripheral blood and chord blood), and bone marrow.

Suitable donor cells may also be in any stage of their individual cellular age, ranging from just separated from their progenitor cell to a senescent or even dead cell. In some embodiments, shedding of microvesicles may be associated with apoptotic blebbing (which may be from the plasma membrane and/or the nucleus). Thus, donor cells may include pre-apoptotic donor cells, or cell committed to apoptosis.

Furthermore, it is contemplated that suitable donor cells also include non-diseased and diseased cells, wherein diseased cells may be affected by one or more pathogens and/or conditions. For example, a diseased donor cell may be infected with a virus, an intracellular parasite, or bacterium. In other examples, a diseased cell may be a metabolically diseased cell (e.g., due to genetic defect, due to an enzyme, receptor, and/or transporter dysfunction, or due to metabolic insult), a neoplastic cell, or cell that has one or more mutations that render the cell susceptible to uncontrolled cell growth. Similarly, donor cells may be native (e.g., obtained by biopsy), cultured (e.g., native, or immortalized), or treated. For example, donor cells may be chemically and/or mechanically treated, resulting in a donor cell that exhibits a cell-specific stress response. In some embodiments, suitable donor cells may be treated with a natural or synthetic ligand to which the cell has a receptor or otherwise complementary structure. In some embodiments, a donor cell may also be treated with a drug or compound that alters at least one of a metabolism, cell growth, cell division, cell structure, and/or secretion.

In some embodiments, suitable donor cells are recombinant cells. For example, recombinant donor cells may contain one or more nucleic acid molecules introduced by recombinant DNA technology. All known manners of introducing nucleic acids are deemed suitable for use herein (e.g., viral transfection, chemical transfection, electroporation, ballistic transfection, etc.). Where the nucleic is a DNA, it is contemplated that the DNA may be integrated into the genome of the donor cell, or that the DNA may reside as extrachromosomal unit within the cell. Such DNA may be employed as a template for RNA production, which may have regulatory and/or protein encoding function. Similarly where the nucleic acid is an RNA, such RNA may be used as a regulatory entity (e.g., via antisense or interference) and/or as a protein encoding entity. As used herein, nucleic acids encompass all known nucleic acid analogs (e.g., phosphorothioate analogs, peptide nucleic acid analogs, etc.)

Suitable donor cells may have any desirable origin, including endothelial, mesothelial, and ectothelial origin. Thus, suitable donor cells include those found in a gland, an organ, muscle, a structural tissue, etc. Suitable donor cells may be heterologous (or non-autologous) or autologous relative to recipient. For example, suitable donor cells may be derived from a tissue the same as or different than the recipient tissue (e.g., a diseased tissue to be treated). As a non-limiting example, microvesicles obtained from donor cells such as fibroblast may be used to treat recipient diseased tissue pancreatic. In some embodiments, donor cells may be derived from a different organism (i.e., non-autologous). For example, a donor cell may be a porcine pancreatic cell, while the recipient is human pancreatic.

In some embodiments, microvesicles are obtained from whole blood, serum, plasma, or any other biological fluid, including urine, ascites fluid, milk, tears, spinal fluid, amniotic fluid, etc., which may be obtained from a living mammal. Alternatively, microvesicles may also be obtained from stored materials (e.g., biological fluids, tissues, organs, etc.). Such storage may include storage at reduced temperature (e.g., 4° C.) or even storage in frozen form. Similarly, microvesicles may also be obtained from an in vitro source, and most typically from cell or tissue culture (see the Cell Culture Condition section below), or even organ culture.

Cell Culture Conditions

In some embodiments, microvesicles are obtained from cultured donor cells. For example, suitable donor cells may be cultured in a liquid medium that contains nutrients for the cells and are incubated in an environment where the temperature and/or gas composition is controlled. As will be appreciated by one of ordinary skill in the art, specific cell culture conditions may vary depending on the type of cells used. For example, cell culture conditions for pathfinder cells have been described. See, e.g., International Patent Publication WO2006120476, the entire contents of which are herein incorporated by reference. An exemplary suitable medium for culture of pathfinder cells contains is CMRL 1066 medium (Invitrogen) supplemented with fetal bovine serum (e.g., at 10%). In some embodiments, media is supplemented with glutamine or glutamine-containing mixtures such as GLUTAMAX™ (Invitrogen) and/or with antibiotics e.g., amphotericin, penicillin, and/or streptomycin).

In some embodiments, cells are grown such they are attached on a surface. In some such embodiments, cells are grown as a monolayer on the surface. In some embodiments, cells are grown until they are confluent, i.e., until they cover the entire surface on which they are growing and there is nowhere else on the surface for cells to grow. In some embodiments, cells are grown until they are close to but not yet at confluence, i.e., until they cover most of the surface on which they are growing, but there is still some room for cells to grow. In some embodiments, cells are grown until they are approximately or more than 50%, 60%, 70%, 80%, 85%, 90%, 95%, 97%, 98%, 99%, or more confluent, wherein x% confluent is defined as coverage of approximately x% of the growing surface. In some embodiments, cells are grown until they are approximately 50-99% (e.g., 60-99%, 70-99%, 75-99%, 80-99%, 85-99%, 90-99%, or 95-99%) confluent.

In some embodiments, cells are grown on a substrate that may affect one or more properties of the cell, such as microvesicle production rate, cell proliferation rate, or miRNA expression pattern. In some embodiments, cells are grown on a nonwoven substrate such as a nonwoven fabric comprised of fibers. As used herein, the term “nonwoven fabric” includes, but is not limited to, bonded fabrics, formed fabrics, or engineered fabrics, that are manufactured by processes other than, weaving or knitting. In some embodiments, the term “nonwoven fabric” refers to a porous, textile-like material, usually in flat sheet form, composed primarily or entirely of fibers, such as staple fibers assembled in a web, sheet or batt. The structure of the nonwoven fabric is based on the arrangement of, for example, staple fibers that are typically arranged more or less randomly. Nonwoven fabrics can be created by a variety of techniques known in the textile industry. Various methods may create carded, wet laid, melt blown, spunbonded, or air laid nonwovens. Exemplary methods and substrates are described in U.S. Application Publication No. 20100151575, the teachings of which are incorporated herein by reference. The density of the nonwoven fabrics may be varied depending upon the processing conditions. In one embodiment, the nonwoven fabrics have a density of about about 60 mg/mL to about 350 mg/mL.

In some embodiments, the nonwoven substrates are biocompatible and/or bioabsorbable. Examples of suitable biocompatible, bioabsorbable polymers that could be used include polymers selected from the group consisting of aliphatic polyesters, poly(amino acids), copoly(ether-esters), polyalkylene oxalates, polyamides, poly(iminocarbonates), polyorthoesters, polyoxaesters, polyamidoesters, polyoxaesters containing amine groups, poly(anhydrides), polyphosphazenes, and blends thereof.

In some embodiments, the aliphatic polyesters are homopolymers and/or copolymers of monomers selected from the group consisting of lactide (which includes lactic acid, and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, delta-valerolactone, beta-butyrolactone, gamma-butyrolactone, epsilon-decalactone, hydroxybutyrate (repeating units), hydroxyvalerate (repeating units), 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof. In another embodiment, aliphatic polyesters which include, but are not limited to homopolymers and/or copolymers of lactide (which includes lactic acid, D-, L- and meso lactide), glycolide (including glycolic acid), epsilon-caprolactone, p-dioxanone (1,4-dioxan-2-one), trimethylene carbonate (1,3-dioxan-2-one) and combinations thereof.

In some embodiments, the aliphatic polyesters are homopolymers and/or copolymers of monomers selected from the group consisting of lactide (which includes lactic acid, D-, L- and meso lactide), glycolide (including glycolic acid), epsilon-caprolactone, p-dioxanone (1,4-dioxan-2-one), trimethylene carbonate (1,3-dioxan-2-one) and combinations thereof. In yet another embodiment, the aliphatic polyesters are homopolymers and/or copolymers of monomers selected from the group consisting of lactide (which includes lactic acid, D-, L- and meso lactide), glycolide (including glycolic acid), and p-dioxanone (1,4-dioxan-2-one) and combinations thereof. Non-limiting examples of suitable fabrics include those that comprise aliphatic polyester fibers, e.g., fibers that comprise homopolymers or copolymers of lactide (e.g., lactic acid D-. L- and meso lactide), glycolide (e.g., glycolic acid), epsilon-caprolactone, p-dioxanone (1,4-dioxan-2-one), trimethylene carbonate (1,3-dioxan-2-one), and combinations thereof. For example, suitable fabrics may contain poly(glycolide-co-lactide) (PGA/PLA), poly(lactide-co-glycolide) (PLA/PGA); 1,3 propanediol (PDO), and/or blends thereof.

In some embodiments, cells are grown on a solid surface that has been textured in a particular way so as to confer special properties to the surface (e.g., repulsion or attraction of certain substances, reduced adsorption of proteins, etc.), which in turn may influence behavior of cells on such surfaces. For example, cells may be grown on a nano-textured surface (“nanosurface”). See, e.g., U.S. Pat. No. 7,597,950; Sun et al. (2009) “Combining nanosurface chemistry and microfluidics for molecular analysis and cell biology,” Analytical Chimica Acta, 650(1):98-105; the entire contents of each of which are herein incorporated by reference. Nanosurfaces and other textured surfaces may be generated, for example by any of a variety of methods known in the art, including sanding, chemical etching, sandblasting, and/or dewetting.

In some embodiments, cells are grown in suspension.

Various growth medium may be used to culture donor cells. Growth medium, generally refers to any substance or preparation used for the cultivation of living cells. In some embodiments, the growth medium is renal growth medium. In some embodiments the growth medium is Dulbecco's Modification of Eagle's medium (DMEM). In some embodiments, cells are grown in media that does not contain serum. In some embodiments, cells are grown for at least a period of time in media that has been depleted of microvesicles from media components. For example, media containing fetal bovine serum may be depleted of bovine microvesicles. Alternatively or additionally, commercially available medium that is depleted of microvesicles (e.g., bovine microvesicles) is used.

In some embodiments, cells are grown at or about 37° C. In some embodiments, cells are grown in the presence of at or about 5% CO₂. In some embodiments, cells are grown under room air oxygen conditions. In some embodiments, cells are grown under conditions where the oxygen pressure is less than or equal to 5% O₂. In some embodiments, cells are grown in conditions of normal oxygen (e.g., about 5% O₂). In some embodiments, cells are grown in hypoxic conditions (e.g., low oxygen such as <5%, <4%, <3%, <2%, or 1% O₂).

In some embodiments, donor cells are grown under serum starvation conditions. As used herein, the term “serum starvation” includes, but is not limited to, serum repletion, serum-free medium or conditions. Various serum starvation conditions are known in the art and can be used to practice the present invention. In some embodiments, cells may be grown under serum starvation conditions for about 6, about 12, about 18, about 24, about 30, about 36, about 42, about 48 hours, or longer. In some embodiments, cells may be grown under conditions where the serum concentration is less than or equal to 10%, less than or equal to 9%, less than or equal to 8%, less than or equal to 7%, less than or equal to 6%, less than or equal to 5%, less than or equal to 4%, less than or equal to 3%, less than or equal to 2%, less than or equal to 1.5%, less than or equal to 1%, or less than or equal to 0.5%. In some embodiments, cells may be grown under conditions where the serum concentration is 0% (i.e., serum is absent). In some embodiments, cells may be grown under conditions where the serum concentration is decreased in a step-wise manner over time. For example, in some embodiments, cells may be grown under conditions where the serum concentration is between about 2% to about 11% (e.g., about 2%, 3%, 4%, 5%, 6%, 7%, 8%, 9%, 10%, or 11%) and is subsequently reduced in one or more steps to a serum concentration between about 0% to about 5% (e.g., about 0%, 0.5%, 1%, 1.5%, 2%, 3%, 4%, or 5%).

Preparation of Microvesicles

Various methods of isolating or enriching microvesicles known in the art may be used to practice the present invention. As used herein, the terms “isolation” or “isolating” in conjunction with microvesicles are interchangeably used with the terms “enrichment” or “enriching,” and refer to one or more process steps that result in an increase of the fraction of microvesicles in a sample as compared to the fraction of microvesicles in the obtained biological sample. Thus, microvesicles may be purified to homogeneity, purified to at least 90% (with respect to non-microvesicle particulate matter), at least 80%, at least 70%, at least 60%, at least 50%, at least 40%, at least 30%, or at least 20% (or even less). For example, physical properties of microvesicles—may be employed to separate them from a medium or other source material. For example, microvesicles may be separated on the basis of electrical charge (e.g., electrophoretic separation), size (e.g., filtration, molecular sieving, etc), density (e.g., regular or gradient centrifugation), Svedberg constant (e.g., sedimentation with or without external force, etc).

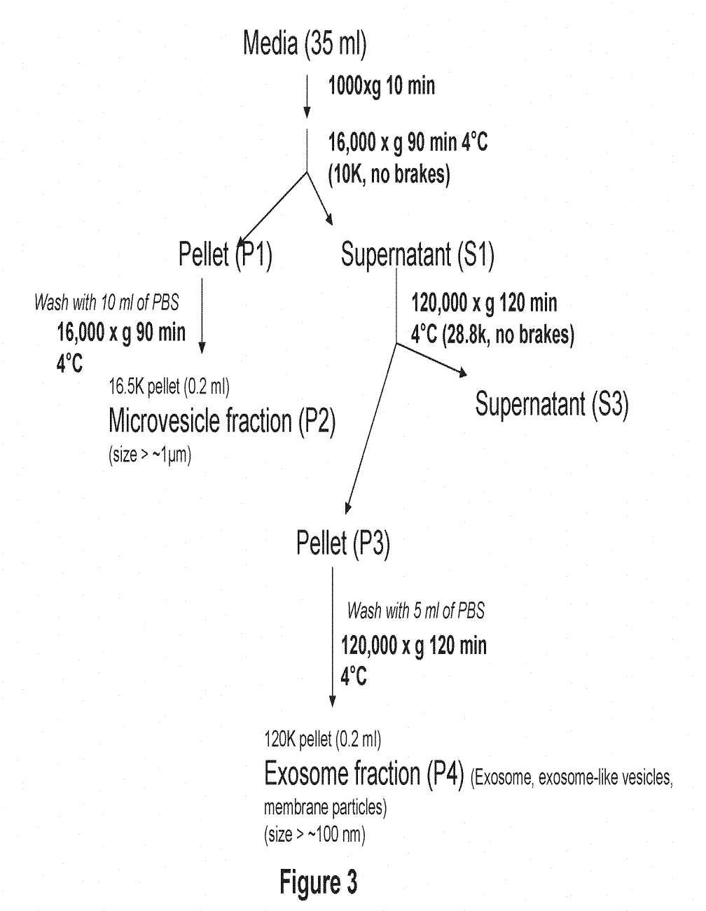

In some embodiments, microvesicles are isolated or purified by centrifugation (e.g., ultracentrifugation). It will be appreciated that various centrifugation conditions (e.g., speed, centrifugal force, centrifugation time, etc.) may be used in order to obtain a desired fraction of isolated or purified microvesicles. For example, in some embodiments, a sample may be centrifuged at a fairly low centrifugal force (e.g., approximately 16,000× g) sufficient to pellet larger microvesicles (e.g., approximately 1000 nm or more). In some embodiments, a sample (e.g., the resulting supernatant from the initial low speed spin) may be centrifuged at a higher centrifugal force (e.g., approximately 120,000× g) sufficient to pellet microvesicles of a smaller size (e.g., less then 1000 nm). In some embodiments, a microvesicle preparation prepared using this method may contain substantially small particles, for example, particles with a size ranging from about 10 nm to 1000 nm (e.g., about 50-1000 nm, 75-1000 nm, 100-1000 nm, 10-750 nm, 50-750 nm, 100-750mn, 100-500 nm), An exemplary microvesicle fractionation schematic is depicted in FIG. 3. In some embodiments, such small particles are also referred to as exosomes, exosome-like vesicles, and/or membrane particles. In some embodiments, such fraction is referred to as exosome fraction.

In some embodiments, microvesicles are isolated or purified by precipitation. It will be appreciated that various precipitation conditions may be used in order to obtain a desired fraction of isolated or purified microvesicles. For example, various kits are available for exosome precipitation, such as ExoQuick™ and Exo-Quick-TC™ (available from System Biosciences, Mountain View, Calif.) and may be used in accordance with the present invention.

Alternatively, or additionally, isolation may be based on one or more biological properties, and may employ surface markers (e.g., for precipitation, reversible binding to solid phase, FACS separation, specific ligand binding, non-specific ligand binding such as annexin V, etc.). In yet further contemplated methods, the microvesicles may also be fused using chemical and/or physical methods, including PEG-induced fusion and/or ultrasonic fusion.

In some embodiments, microvesicles are obtained from conditioned media from cultures of microvesicle-producing cells.

Synthetic Microvesicles

In some embodiments, microvesicles suitable for the present invention may be synthetically produced. Synthetic microvesicles typically include one or more membrane components obtained from a donor cell. In some embodiments, synthetic microvesicles include at least one microRNA described herein. For example, synthetic microvesicles may be prepared by disintegration of a donor cell (e.g., via detergent, sonication, shear forces, etc.) arid use of the crude preparation or an at least partially enriched membrane fraction to reconstitute one or more microvesicles. In some embodiments, exogenous microRNAs may be added to microvesicles.

IL Micro RNAs

In some embodiments, microvesicles comprise one or more specific microRNAs. As used herein, microvesicle-specific microRNAs include those microRNAs only present in microvesicles not in cells and those microRNAs that are substantially enriched in microvesicles as compared to cells. Microvesicle-specific microRNAs encompass microRNAs isolated or purified from microvesicles or synthesized using recombinant or chemical techniques. For example, microRNA molecules may be generated by in vitro transcription of DNA sequences encoding the relevant molecule. Such DNA sequences may be incorporated into a wide variety of vectors with suitable RNA polymerase promoters such as T7, T3, or SP6. As used herein, the term “microRNAs (miRNAs)” refers to post-transcriptional regulators that typically bind to complementary sequences in the three prime untranslated regions (3′ UTRs) of target messenger RNA transcripts (mRNAs), usually resulting in gene silencing. Typically, miRNAs are short ribonucleic acid (RNA) molecules. For example, microRNAs may be approximately 18-25 nucleotides long (e.g., approximately 18, 19, 20, 21, 22, 23, 24 or 25 nucleotides long).

It is contemplated that microvesicle specific microRNAs, individually or in combination, may be used to induce or stimulate tissue or cell growth, remodeling, reconstruction, differentiation and/or transdifferentiation, among other functions. Thus, the present invention provides, among other things, methods of identifying microvesicle-specific microRNAs or any microRNAs that can induce or stimulate tissue or cell growth, remodeling, reconstruction, differentiation and/or transdifferentiation.

In some embodiments, inventive methods according to the present invention may include one or more of the following steps of: providing cells grown in a microvesicle-depleted medium, adding an miRNA to the medium, and determining if addition of the miRNA increases cell proliferation rate as compared to a control, thereby identifying if the miRNA induces cell growth and/or regeneration. In some embodiments, doubling time (e.g., the time it takes to double the population of cells in a cell culture vessel) is used as an indication of cell proliferation rate.

Cell proliferation assays are known in the art, and any of a variety of such assays may be employed to determine cell proliferation rates. For example, cell numbers (e.g., per volume of media; or for an entire cell culture vessel, etc.) may be counted using standard cell counting techniques known in the art. In some such cell counting methods, cells are labeled with a dye to ease detection. In some methods of assessing cell proliferation, cells are brought into a suspension of a known volume and the density (e.g., optical density) of at least an aliquot of the cell suspension is measured using standard spectrophotometry techniques.

Some cell proliferation assays measure DNA synthesis. For example, incorporation of a labeled nucleotide or nucleotide analog (e.g., BrdU (bromodeoxyuridine) tritium-labeled thymidine, etc. can be employed in a cell proliferation assay. Some cell proliferation assays measure conversion of a substrate by a metabolic enzyme. For example, an “MTT” assay measures the cleavage of a tetrazolium salt WST-1 to formazan by cellular mitochondrial dehydrogenases.

In some embodiments, cell viability is also measured and taken into account such that only viable cells are counted. For example, the ability to exclude trypan blue dye is taken as a sign of membrane integrity and therefore cell viability, and cell counting methods typically include using trypan blue.

In some embodiments, inventive methods for identifying microRNA according to the present invention may include one or more of the following steps of: creating a wounded area in cells grown to confluence; treating the cells with an miRNA; and determining a rate of re-growth of the treated cells across the wounded area as compared to a control, thereby identifying if the miRNA induces cell growth and/or regeneration.

Re-growth over wounded areas in a confluent cell culture can be measured by methods known in the art. In some embodiments, re-growth is measured quantitatively. For example, re-growth can be measured quantitatively using, e.g., an XCELLIGENCE™ System (Roche Applied Science)

In some embodiments, methods are performed in a high-throughput fashion, e.g., with many miRNAs being tested in parallel. Multi-well plates (e.g., 24-well, 48-well. 96-well, 324-well, etc.) may facilitate such parallel testing, as each miRNA may be tested in an individual well.

Any type of cells that can be grown in culture can be used in methods of the invention. For example, various donor cells described herein may be used. In some embodiments, suitable cells include pancreas-derived pathfinder cells, fibroblasts, and cardiomyocytes.

Various candidate miRNAs may be tested using inventive methods described herein. For example, miRNAs that are isolated from microvesicles may be used. Alternatively or additionally, miRNAs that have been identified in the literature or in other experiments as being of potential interest (e.g., as being associated with a disease, with transdifferentiation, with potential therapeutic applications, etc.) may be used in methods of the invention to determine of such miRNAs induce cell growth and/or regeneration. In some embodiments, a miRNA library is used. For example, a collection of cloned miRNAs from an expression library may be used in accordance with methods of the invention to identify one or more miRNAs that induce cell growth and/or regeneration. In some embodiments, an miRNA expression library from a cell type of interest is used.

Appropriate controls in the step of determining include, but are not limited to, untreated cells that are otherwise grown under identical conditions (e.g., cells to which no miRNA is added), and/or cells to which a “control” miRNA is added that are otherwise grown under identical conditions. The “control” miRNA, if used, generally has a known effect on cell growth and/or regeneration. In some embodiments, more than one control is used. In some embodiments, a negative control (one for which no inducement of cell growth and/or regeneration is expected) is used. In some embodiments, a positive control (one for which inducement of cell growth and/or regeneration is expected) is used. In some embodiments, both a positive and negative control is used.

Table 1 shows exemplary microRNAs that are specifically present in microvesicles. In some embodiments, it was found that miRNA-122, miRNA-127, miRNA-133b, miRNA-323, miRNA-433, miRNA-451, miRNA-466h, miRNA-467c, miRNA-467e, miRNA-468, miRNA-491, miRNA-495, miRNA-546, miRNA-666, miRNA-680, and miRNA-346 (SEQ ID NOs:1-29) are present in microvesicles at relatively higher concentrations. Additional microRNAs identified according to the present invention are listed in Tables 3-13. Table 1 lists exemplary miRNA sequences for each miRNA of interest; corresponding miRNA sequences in other species, including, but not limited to, Homo sapiens, Rattus norvegicus, Mus musculus, Danio rerio, and Gallus gallus, are publicly available (e.g., see http://diana.cslab.ece.ntua.gr/mirgen1). As can be seen in Table 1 and Tables 7-13, some miRNA sequences are well conserved across species, and some miRNA sequence variants exist even in the same species. Tables 7-13 show exemplary microRNAs that may be used in accordance with the present invention.

TABLE 1
microRNA sequences

| | Sequence |
|---|---|
| microRNA | (species, variant (if applicable)) |
| miR122 | UGGAGUGUGACAAUGGUGUUUG (SEQ ID NO: 1) |
| | (Homo sapiens) |
| | UGGAGUGUGACAAUGGUGUUUG (SEQ ID NO: 2) |
| | (Rattus norvegicus) |
| miR127 | CUGAAGCUCAGAGGGCUCUGAU (SEQ ID NO: 3) |
| | (Homo sapiens, miR127-5p) |
| | UCGGAUCCGUCUGAGCUUGGCU (SEQ ID NO: 4) |
| | (Homo sapiens, miR127-3p) |
| | UCGGAUCCGUCUGAGCUUGGCU (SEQ ID NO: 5) |
| | (Rattus norvegicus) |
| miR133b | UUUGGUCCCCUUCAACCAGCUA (SEQ ID NO: 6) |
| | (Homo sapiens) |
| | UUUGGUCCCCUUCAACCAGCUA (SEQ ID NO: 7) |
| | (Rattus norvegicus) |
| miR323 | AGGUGGUCCGUGGCGCGUUCGC (SEQ ID NO: 8) |
| | (Homo sapiens, miR323-5p) |
| | CACAUUACACGGUCGACCUCU (SEQ ID NO: 9) |
| | (Homo sapiens, miR323-3p) |
| | CACAUUACACGGUCGACCUCU (SEQ ID NO: 10) |
| | (Rattus norvegicus) |
| | AGGUGGUCCGUGGCGCGUUCGC (SEQ ID NO: 11) |
| | (Rattus norvegicus, variant) |
| miR346 | UGUCUGCCCGCAUGCCUGCCUCU (SEQ ID NO: 12) |
| | (Homo sapiens) |
| | UGUCUGCCUGAGUGCCUGCCUCU (SEQ ID NO: 13) |
| | (Rattus norvegicus) |
| miR433 | AUCAUGAUGGGCUCCUCGGUGU (SEQ ID NO: 14) |
| | (Homo sapiens) |
| | AUCAUGAUGGGCUCCUCGGUGU (SEQ ID NO: 15) |
| | (Rattus norvegicus) |
| miR451 | AAACCGUUACCAUUACUGAGUU (SEQ ID NO: 16) |
| | (Homo sapiens) |
| | AAACCGUUACCAUUACUGAGUU (SEQ ID NO: 17) |
| | (Rattus norvegicus) |
| miR466h | UGUGUGCAUGUGCUUGUGUGUA (SEQ ID NO: 18) |
| | (Mus musculus) |
| miR467c | UAAGUGCGUGCAUGUAUAUGUG (SEQ ID NO: 19) |
| | (Mus musculus) |
| miR467e | AUAAGUGUGAGCAUGUAUAUGU (SEQ ID NO: 20) |
| | (Mus musculus) |
| | AUAUACAUACACACACCUAUAU (SEQ ID NO: 21) |
| | (Mus musculus, variant) |
| miR468 | UAUGACUGAUGUGCGUGUGUCUG (SEQ ID NO: 22) |
| | (Mus musculus) |
| miR491 | AGUGGGGAACCCUUCCAUGAGG (SEQ ID NO: 23) |
| | (Homo sapiens, miR491-5p) |
| | CUUAUGCAAGAUUCCCUUCUAC (SEQ ID NO: 24) |
| | (Homo sapiens, miR491-3p) |
| miR495 | AAACAAACAUGGUGCACUUCUU (SEQ ID NO: 25) |
| | (Homo sapiens) |
| | AAACAAACAUGGUGCACUUCUU (SEQ ID NO: 26) |
| | (Rattus norvegicus) |
| miR546 | AUGGUGGCACGGAGUC (SEQ ID NO: 27) |
| | (Mus musculus) |
| miR666 | AGCGGGCACGGCUGUGAGAGCC (SEQ ID NO: 28) |
| | (Rattus norvegicus) |
| miR680 | GGGCAUCUGCUGACAUGGGGG (SEQ ID NO: 29) |
| | (Mus musculus) |
| miR136 | ACUCCAUUUGUUUUGAUGAUGGA (SEQ ID NO: 30) |
| | (Homo sapiens) |
| | CAUCAUCGUCUCAAAUGAGUCU (SEQ ID NO: 31) |
| | (Homo sapiens, variant) |
| miR202 | AGAGGUAUAGGGCAUGGGAA (SEQ ID NO: 32) |
| | (Homo sapiens) |
| | UUCCUAUGCAUAUACUUCUUUG (SEQ ID NO: 33) |
| | (Homo sapiens, variant) |
| | UUCCUAUGCAUAUACUUCUUU (SEQ ID NO: 34) |
| | (Rattus norvegicus) |
| miR206 | UGGAAUGUAAGGAAGUGUGUGG (SEQ ID NO: 35) |
| | (Homo sapiens) |
| | UGGAAUGUAAGGAAGUGUGUGG (SEQ ID NO: 36) |
| | (Rattus norvegicus) |
| miR224 | CAAGUCACUAGUGGUUCCGUU (SEQ ID NO: 37) |
| | (Homo sapiens) |
| | AAAAUGGUGCCCUAGUGACUACA (SEQ ID NO: 38) |
| | (Homo sapiens, variant) |
| | CAAGUCACUAGUGGUUCCGUUUA (SEQ ID NO: 39) |
| | (Rattus norvegicus) |
| miR327 | CCUUGAGGGGCAUGAGGGU (SEQ ID NO: 40) |
| | (Rattus norvegicus) |
| miR347 | UGUCCCUCUGGGUCGCCCA (SEQ ID NO: 41) |
| | (Rattus norvegicus) |
| miR369 | AGAUCGACCGUGUUAUAUUCGC (SEQ ID NO: 42) |
| | (Homo sapiens, miR369-5p) |
| | AAUAAUACAUGGUUGAUCUUU (SEQ ID NO: 43) |
| | (Homo sapiens, miR369-3p) |
| | AGAUCGACCGUGUUAUAUUCGC (SEQ ID NO: 44) |
| | (Rattus norvegicus, miR369-5p) |
| | AAUAAUACAUGGUUGAUCUUU (SEQ ID NO: 45) |
| | (Rattus norvegicus, miR369-3p) |
| miR370 | GCCUGCUGGGGUGGAACCUGGU (SEQ ID NO: 46) |
| | (Homo sapiens) |
| | GCCUGCUGGGGUGGAACCUGGUU (SEQ ID NO: 47) |
| | (Rattus norvegicus) |
| miR375 | UUUGUUCGUUCGGCUCGCGUGA (SEQ ID NO: 48) |
| | (Homo sapiens) |
| | UUUGUUCGUUCGGCUCGCGUGA (SEQ ID NO: 49) |
| | (Rattus norvegicus) |
| miR376b | AUCAUAGAGGAAAAUCCAUGUU (SEQ ID NO: 50) |
| | (Homo sapiens) |
| | GUGGAUAUUCCUUCUAUGGUUA (SEQ ID NO: 51) |
| | (Rattus norvegicus, miR376-5p) |
| | AUCAUAGAGGAACAUCCACUU (SEQ ID NO: 52) |
| | (Rattus norvegicus, miR376-3p) |
| miR381 | UAUACAAGGGCAAGCUCUCUGU (SEQ ID NO: 53) |
| | (Homo sapiens) |
| | UAUACAAGGGCAAGCUCUC (SEQ ID NO: 54) |
| | (Rattus norvegicus) |
| miR434 | UUUGAACCAUCACUCGACUCCU (SEQ ID NO: 55) |
| | (Rattus norvegicus) |
| miR452 | AACUGUUUGCAGAGGAAACUGA (SEQ ID NO: 56) |
| | (Homo sapiens) |
| | CUCAUCUGCAAAGAAGUAAGUG (SEQ ID NO: 57) |
| | (Homo sapiens, variant) |
| miR465a | UAUUUAGAAUGGCACUGAUGUGA (SEQ ID NO: 58) |
| | (Mus musculus, miR465a-5p) |
| | GAUCAGGGCCUUUCUAAGUAGA (SEQ ID NO: 59) |
| | (Mus musculus, miR465-3p) |
| miR465b | UAUUUAGAAUGGUGCUGAUCUG (SEQ ID NO: 60) |
| | (Mus musculus, miR465b-5p) |
| | GAUCAGGGCCUUUCUAAGUAGA (SEQ ID NO: 61) |
| | (Mus musculus, miR465b-3p) |
| miR470 | UUCUUGGACUGGCACUGGUGAGU (SEQ ID NO: 62) |
| | (Mus musculus) |
| | AACCAGUACCUUUCUGAGAAGA (SEQ ID NO: 63) |
| | (Mus musculus, variant) |
| miR487b | AAUCAUACAGGGACAUCCAGUU (SEQ ID NO: 64) |
| | (Homo sapiens) |
| miR543 | AAACAUUCGCGGUGCACUUCUU (SEQ ID NO: 65) |
| | (Homo sapiens) |
| | AAGUUGCCCGCGUGUUUUUCGC (SEQ ID NO: 66) |
| | (Rattus norvegicus) |
| | AAACAUUCGCGGUGCACUUCU (SEQ ID NO: 67) |
| | (Rattus norvegicus, variant) |
| miR547 | UUGGUACUUCUUUAAGUGAG (SEQ ID NO: 68) |
| | (Rattus norvegicus) |
| miR590 | GAGCUUAUUCAUAAAAGUGCAG (SEQ ID NO: 69) |
| | (Homo sapiens, miR590-5p) |
| | UAAUUUUAUGUAUAAGCUAGU SEQ ID NO: 70) |
| | (Homo sapiens, miR590-3p) |
| miR741 | UGAGAGAUGCCAUUCUAUGUAGA (SEQ ID NO: 71) |
| | (Mus musculus) |
| miR881 | AACUGUGGCAUUUCUGAAUAGA (SEQ ID NO: 72) |
| | (Rattus norvegicus) |

It is contemplated that one or more microRNAs identified according to the present invention (e.g., SEQ ID NOs. 1-72 and those listed in Tables 7-13, may be used to induce or stimulate tissue or cell growth, remodeling, reconstruction, differentiation and/or transdifferentiation, and/or to treat associated diseases, disorders or conditions. In some embodiments, functional variants of microRNAs described herein may be used. For example, suitable microRNAs may include microRNAs having a sequence at least 70% (e.g., 75%, 80%, 85%, 90%, 95%, 96%, 97%, 98%, 99%) identical to any one of microRNAs identified in Table 1 and Tables 7-13. In some embodiments, suitable microRNAs are functional variants of microRNAs that are present at a relatively higher concentration in microvesicles. Accordingly, in some embodiments, suitable microRNAs may include microRNAs having a sequence at least 70% (e.g., 75%, 80%, 85%, 90%, 95%, 96%, 97%, 98%, 99%) identical to any one of SEQ ID NO:1 to 16,

“Percent (%) nucleic acid sequence identity” with respect to microRNA sequences identified herein is defined as the percentage of nucleotides in a candidate sequence that are identical with the nucleotides in a reference sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology, 266, 460-480 (1996); http://blastwust1/edu/blast/README.html). WU-BLAST-2 uses several search parameters, most of which are set to the default values. The adjustable parameters are set with the following values: overlap span=1, overlap fraction=0.125, world threshold (T)=11. HSP score (5) and HSP S2 parameters are dynamic values and are established by the program itself, depending upon the composition of the particular sequence, however, the minimum values may be adjusted and are set as indicated above.

Suitable microRNAs may be comprised entirely of natural RNA nucleotides, or may instead include one or more nucleotide analogs and/or modifications. The microRNA structure may be stabilized, for example by including nucleotide analogs at one or more free strand ends in order to reduce digestion, e.g., by exonucleases. Suitable microRNAs may contain modified ribonucleotides, that is, ribonucleotides that contain a modification in the chemical structure of an unmodified nucleotide base, sugar and/or phosphate (or phospodiester linkage). As is known in the art, an “unmodified ribonucleotide” has one of the bases adenine, cytosine, guanine, and uracil joined to the 1′ carbon of beta-D-ribo-furanose. Modified microRNA molecules may also contain modified backbones or non-natural intemucleoside linkages, e.g., modified phosphorous-containing backbones and non-phosphorous backbones such as morpholino backbones; siloxane, sulfide, sulfoxide, sulfone, sulfonate, sulfonamide, and sulfatnate backbones; formacetyl and thioformacetyl backbones; alkene-containing backbones; methyleneimino and methylenehydrazino backbones; amide backbones, and the like.

### III. Therapeutic Applications

In some embodiments, the present invention provides methods of using microvesicles and/or microRNAs for inducing or stimulating tissue or cell growth, remodeling, reconstruction, differentiation and/or transdifferentiation, or treating associated diseases, disorders or conditions. While not wishing to be bound by a particular theory or hypothesis, it is contemplated that microvesicles may induce changes within target tissue or cells to convert them into active repair mode by providing microRNAs and/or other components (e.g., membrane associated polypeptide, transcription factors, etc.) that will regulate expression of genes relating to, e.g., increased cell mobility, tissue remodeling and reprogramming, growth, angiogenesis, cell adhesion and cell signaling, etc. It is further contemplated that microvesicles will typically not be part of the new tissue or cells. Thus, according to the present invention, microvesicles or microRNAs from different tissues, cell types or organisms may be used. In some embodiments, microvesicles or microRNAs may be used without inducing immuno reaction. In some embodiments, microvesicles or microRNAs may be used without an immunosuppressant.

Thus, suitable microvesicles or microRNAs can be derived from autologous cells (i.e., cells from the same individual as the patient) or non-autologous cells (i.e., cells from a different individual as the patient) or both. In some embodiments, microvesicles are derived from tissue that is the same as the diseased tissue. For example, in methods of treating a kidney disease, microvesicles may be taken from healthy kidney cells from the same or different individual being treated. In some embodiments, microvesicles are derived from tissue that is different than the diseased tissue.

In some embodiments, methods of treatment comprise one or more steps that are performed in vitro or ex vivo to induce cells (“recipient cells”) to differentiate or transdifferentiate into a desirable cell type. Such recipient cells can then be transferred into a patient.

In some embodiments, provided methods comprise co-culturing donor cells (i.e., cells that produce microvesicles) and recipient cells (i.e., cells that received microvesicles and/or contents of such microvesicles) ex vivo and then transferring recipient cells into an patient. In some embodiments, recipient cells are transferred back into the same individual from whom recipient cells were obtained. For example, pathfinder cells can be co-cultured with bone marrow cells obtained from an patient for a period of time ex vivo to allow transfer of microvesicles and/or their contents, then bone marrow cells may be transferred back into the individual.

In some embodiments, recipient cells are tested for expression of specific biomarkers such as certain microRNAs after co-culturing with donor cells before transfer into a patient.

In certain embodiments, methods of treatment comprise a step of administering to a patient in need of treatment a therapeutically effective amount of one or more microRNAs as described herein. miRNAs may be used in the absence or presence of microvesicles or derivatives thereof.

In some embodiments, methods and compositions (e.g., microvesicles and/or microRNAs) according to the present invention may be used to treat diseases, disorders, or conditions in various tissues including, but not limited to, central nervous system (CNS), peripheral nervous system, cardiovascular system, respiratory system, gastrointestinal tract and associated glands, integumentary system, musculoskeletal system, and other systems of the body. In some embodiments, methods and compositions (e.g., microvesicles and/or microRNAs) according to the present invention may be used to treat age-related degeneration. In some embodiments, methods and compositions (e.g., microvesicles and/or microRNAs) according to the present invention may be used to treat inflammation. In some embodiments, microvesicles and/or microRNAs according to the present invention may be suitable for cosmetic uses or for treating a condition or disorder associated with a cosmetic surgical procedure.

Inflammation

In some embodiments, methods and compositions of the present invention are used to treat or ameliorate inflammation. As used herein, the term “inflammation” includes inflammatory conditions occurring in many disorders which include, but are not limited to: Systemic inflammatory Response (SIRS); Alzheimer's Disease and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1. induced plasticity of hypothalamic neurons and chronic stress hyper responsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media. (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosotnes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

In particular embodiments, methods and compositions of the present invention can be used to treat or ameliorate inflammation associated with an immunodeficiency disease, disorder, or condition. Non-limiting examples of diseases, disorders, and conditions that may be characterized by immunodeficiency include hypgammaglobulinemia, agammaglobulinemia, ataxia telengiectasia, severe combined immunodeficiency disease (SCID), acquired immunodeficiency syndrome (AIDS) such as that caused by infection by human immunodeficiency virus (HIV), Chediak-Higashi syndrome, combined immunodeficiency disease, complement deficiencies, diGeorge syndrome, Job syndrome, leukocyte adhesion defects, panhypogammaglobulinemia (e.g., Bruton disease, congential agammaglobulinemia, selective deficiency of IgA, Wiscott-Aldrich syndrome. In some embodiments, pathfinder cells and/or cells differentiated from pathfinder cells treat or ameliorate immunodeficiency by stimulating reconstitution of one or more blood cell types, i.e., cells of the immune system. It is contemplated that pathfinder cell-associated microRNAs disclosed herein would similarly be useful in treating or ameliorating immunodeficiency.

In certain embodiments, methods and compositions of the present invention are used to treat or ammeliorate an autoimmune diesase, disorder or condition. In general, autoimmunity is the failure of an organism to recognize its own constituent parts as “self,” which results in an immune response against the organism's own tissues and cells. Exemplary autoimmune diseases and/or suspected autoimmune diseases include, but are not limited to, Acute disseminated encephalomyelitis (ADEM), Addison's disease, Alopecia universalis, Ankylosing spondylitisis, Antiphospholipid antibody syndrome (APS), Aplastic anemia, Autoimmune hemolytic anemia, Autoimmune hepatitis, Autoimmune inner ear disease (AIED), Autoimmune lymphoproliferative syndrome (ALPS), Autoimmune oophoritis, Balo disease, Behcet's disease, Bullous pemphigoid, Cardiomyopathy, Chagas' disease, Chronic fatigue immune dysfunction syndrome (CMS), Chronic inflammatory demyelinating polyneuropathy, Crohn's disease, Cicatrical pemphigoid, Coeliac sprue-dermatitis herpetiformis, Cold agglutinin disease, CREST syndrome, Degos disease, Diabetes mellitus, Discoid lupus, Dysautonomia, Endometriosis, Essential mixed cryoglobulinetnia, Fibromyalgia-fibromyositis, Goodpasture's syndrome, Grave's disease, Guillain-Barré syndrome (GBS), Hashimoto's thyroiditis, Hidradenitis suppurativa, Idiopathic and/or acute thrombocytopenic purpura, idiopathic pulmonary fibrosis, IgA neuropathy, Interstitial cytisis, Juvenile arthritis, Kawasaki's disease, Lichen planus, Lupus erythematosus, Lyme disease, Ménière disease, Mixed connective tissue disease (MCTD), Multiple sclerosis, Myasthenia gravis, Neuromyotonia, Opsoclonus myoclonus syndrome (OMS), Optic neuritis, Ord's thyroiditis, Osteoarthritis, Pemphigus vulgaris, Pernicious anemia, Polyarthritis, Polychondritis, Polymyositis and dermatomyositis, Primary biliary cirrhosis, Psoriasis, Polyarteritis nodosa, Polyglandular syndromes, Polymyalgia rheumatica, Primary agammaglobulinemia, Raynaud phenomenon, Reiter's syndrome, Rheumatic fever, Sarcoidosis, Schizophrenia, Scleroderma, Sjogren's syndrome, Stiff person syndrome, Takayasu's arteritis, Temporal arteritis (also known as “giant cell arteritis”), Ulcerative colitis, Uveitis, Vasculitis, Vitiligo, Vulvodynia (“vulvar vestibulitis”), and Wegener's granulomatosis.

Transplantation Stress

In certain embodiments, methods and compositions of the present invention are used to alleviate transplantation stress. It is contemplated that tissue/organ transplantation may cause acute tissue damage and microvesicles disclosed herein may be administered into an organ/tissue transplant recipient to stimulate tissue repair, regeneration, reconstitution, remodeling, and/or inducing immune tolerance, thereby alleviating transplantation stress. It is contemplated that the present invention may be used to facilitate any organ transplantation including, but not limited to, heart, kidney, liver, lung, pancreas, intestine, thymus, and skin transplantation.

In certain embodiments, methods and compositions of the present invention are used to treat or ameliorate a disease, disorder, or condition associated with graft rejection. In general, graft rejection may result from functional immune cells in a recipient recognizing a donor organ or tissue as a foreign entity and mounting of an immunologic attack on the donor organ or tissue. In some cases, graft rejection arises in an acute phase following transplantation of donor organs or tissues to a recipient. In some cases, graft rejection arises in a chronic phase following transplantation of donor organs or tissues to a recipient. It is to be understood that the present invention encompasses methods and compositions for treatment of acute and/or chronic graft rejection.

In certain embodiments, methods and compositions of the present invention are used to treat or ameliorate a graft versus host disease, disorder, or condition. In general, Graft versus Host disease (GI/HD) may result from functional immune cells in a transplanted tissue or organ from a donor recognizing the recipient as a foreign entity and mounting an immunologic attack on the recipient's cells and/or tissues. In some cases, GVHD arises in an acute phase following transplantation of donor organs or tissues to a recipient. In some cases, GVHD arises in a chronic phase following transplantation of donor organs or tissues to a recipient. It is to be understood that the present invention encompasses methods and compositions for treatment of acute and/or chronic GVHD.

Immune Tolerance

It is contemplated that pathfinder cells or their extracellular secretomes (e.g., microvesicles) induce immune tolerance and thus are particularly useful in treating inflammation and suppressing, inhibiting or reducing transplantation associated stress. Without wishing to be bound by particular theory, it is contemplated that the pathfinder cells or their extracellular secretomes (e.g., microvesicles) induce immune tolerance by inducing increased IL-2 response, resulting in expansion of regulatory T cells (e.g., increased level and/or activity of T regulatory cells), decreased level and/or activity of cytotoxic T cells and/or helper I cells, and/or suppression of T cell or non T cell lymphocyte responses. In some embodiments, pathfinder cells or their extracellular secretomes (e.g., microvesicles) suppress pro-inflammatory and/or anti-angiogenic cytokine or chemokine response. Pro-inflammatory and/or anti-angiogenic cytokines or chemokines are well known in the art. Exemplary pro-inflammatory and/or anti-angiogenic cytokines or chemokines include, but are not limited to, IL-4, IL-5, IL-10, IL-12, IL-13, GMCSF, TGF-β, TNF-α, IFN-γ, MCAF, and MIP1. In some embodiments, cells or their extracellular secretomes (e.g., microvesicles) increase anti-inflammatory and/or pro-angiogenic cytokine or chemokine response. Anti-inflammatory and/or pro-angiogenic cytokines or chemokines are known in the art. Exemplary anti-inflammatory and/or pro-angiogenic cytokines or chemokines include, but are not limited to, IL-1β, GSCF, and IL-8,

Accordingly, administration of pathfinder cells or their extracellular secretomes (e.g., microvesicles) according to the present invention does not result in severe adverse effects in the subject. As used herein, severe adverse effects include, but are not limited to, substantial immune response, toxicity, or death. As used herein, the term “substantial immune response” refers to severe or serious immune responses, such as adaptive T-cell immune responses.

Thus, in many embodiments, inventive methods according to the present invention do not involve concurrent immunosuppressant therapy (i.e., any immunosuppressant therapy used as pre-treatment/pre-conditioning or in parallel to the method). In some embodiments, inventive methods according to the present invention do not involve an immune tolerance induction in the subject being treated. In some embodiments, inventive methods according to the present invention do not involve a pre-treatment or preconditioning of the subject using T-cell immunosuppressive agent.

In some embodiments, however, administration of pathfinder cells or their extracellular secretomes e.g., microvesicles) according to the present invention can mount an immune response against these agents. Thus, in some embodiments, it may be useful to render the subject receiving the cells or their extracellular secretomes (e.g., microvesicles) tolerant to the therapy. Immune tolerance may be induced using various methods known in the art. Any immunosuppressant agent known to the skilled artisan may be employed together with a combination therapy of the invention. Such immunosuppressant agents include but are not limited to cyclosporine, FK506, rapamycin, CTLA4-Ig, and anti-TNF agents such as etanercept (see e.g. Moder, 2000, Ann. Allergy Asthma Immunol. 84, 280-284; Nevins, 2000, Curr. Opin. Pediatr. 12, 146-150; Kuriberg et al., 2000, Scand, J. Immunol. 51, 224-230; Ideguchi et al., 2000, Neuroscience 95, 217-226; Potteret al., 1999, Ann. N.Y. Acad. Sci. 875, 159-174; Slavik et al., 1999, Immunol. Res. 19, 1-24; Gaziev et al., 1999, Bone Marrow Transplant. 25, 689-696; Henry, 1999, Clin. Transplant. 13, 209-220; Gummert et al., 1999, J. Am. Soc, Nephrol. 10, 1366-1380; Qi et al., 2000, Transplantation 69, 1275-1283). The anti-IL2 receptor (alpha.-subunit) antibody daclizumab (e.g. Zenapax™), which has been demonstrated effective in transplant patients, can also be used as an immunosuppressant agent (see e.g. Wiseman et al., 1999, Drugs 58, 1029-1042; Beniaminovitz et al., 2000, N. Engl. J. Med. 342, 613-619; Ponticelli et al., 1999, Drugs R. D. 1, 55-60; Berard et al., 1999, Pharmacotherapy 19, 1127-1137; Eckhoff et al., 2000, Transplantation 69, 1867-1872; Ekberg et al., 2000, Transpl. Int. 13, 151-159). Additional immunosuppressant agents include but are not limited to anti-CD2 (Branco et al., 1999, Transplantation 68, 1588-1596; Przepiorka et al., 1998, Blood 92, 4066-4071), anti-CD4 (Marinova-Mutafchieva et al., 2000, Arthritis Rheum. 43, 638-644; Fishwild et al., 1999, Clin. Immunol. 92, 138-152), and anti-CD40 ligand (Hong et al., 2000, Semin. Nephrol. 20, 108-125; Chirmule et al., 2000, J. Virol. 74, 3345-3352; Ito et al., 2000, J. Immunol. 164, 1230-1235).

In addition, methods and compositions pathfinder cells, cells differentiated from pathfinder cells, microvesicles and/or microRNAs) according to the present invention may be used to treat diseases, disorders, or conditions in various tissues including, but not limited to, central nervous system (CNS), peripheral nervous system, cardiovascular system, respiratory system, gastrointestinal tract and associated glands, integumentary system, musculoskeletal system, and other systems of the body. In some embodiments, methods and compositions according to the present invention may be used to treat age-related degeneration as well as progerias. In some embodiments, methods and compositions according to the present invention may be used to treat inflammation. In some embodiments, cells and/or microRNAs according to the present invention may be suitable for cosmetic uses or for treating a condition or disorder associated with a cosmetic surgical procedure.

Central Nervous System (CNS)

Examples of CNS-related diseases, disorders or conditions that may be treated by the methods and compositions of the present invention include motor neurone disease, multiple sclerosis, degenerative diseases of the CNS, dementive illnesses such as Alzheimer's disease, age related dysfunction of the CNS, Parkinson's disease, cerebrovascular accidents, epilepsy, temporary ischaemic accidents, disorders of mood, psychotic illnesses, specific lobe dysfunction, pressure related injury, cognitive dysfunction or impairments, deafness, blindness anosmia, diseases of the special senses, motor deficits, sensory deficits, head injury and trauma to the CNS. Methods and products of the present invention may also be used to enhance brain function or ameliorate deficiencies at a functional level or to facilitate post surgical repair of the CNS.

Cardiovascular System

Examples of diseases, disorders or conditions of the cardiovascular system that may be treated by the methods and compositions of the present invention include arrhythmias, myocardial infarction and other heart attacks, pericarditis, congestive heart diseases, valve-related pathologies, myocardial, endocardial and pericardial dysfunctions or degeneration, age-related cardiovascular disorders, dysfunctions, degeneration or diseases, sclerosis and thickening of valve flaps, fibrosis of cardiac muscle, decline in cardiac reserve, congenital defects of the heart or circulatory system, developmental defects of the heart or circulatory system, repair of hypoxic or necrotic damage, blood vessel damage and cardiovascular diseases or dysfunction (e.g., angina, dissected aorta, thrombotic damage, aneurysm, atherosclerosis, emboli damage and other problems associated with blood flow, pressure or impediment). Methods and compositions of the present invention may also be used to enhance cardiovascular function or health and to revascularise tissues. Moreover, methods and compositions of the present invention may be used to repair, modify, enhance or regenerate traumatic damage to the heart or blood vessels and as a technique to enhance the transplantation/implantation of a whole organ or its parts. Examples of this latter embodiment include heart transplantation, valve replacement surgeries, implantation of prosthetic devices and the development of novel surgical techniques.

Respiratory System

Examples of diseases, disorders or conditions of the respiratory system that may be treated by the methods and compositions of the present invention include damage, pathology, ageing and trauma of the nose and paranasal sinuses, nasopharynx, oropharynx, laryngopharynx, larynx, vocal ligaments, vocal cords, vestibular folds, glottis, epiglottis, trachea, mucocilliary mucosa, trachealis muscle, primary bronchi, lobar bronchi, segmental bronchi, terminal bronchioles, respiratory zone structures and plural membranes. Examples of such damage include obstructive pulmonary diseases, restrictive disorders, emphysema, chronic bronchitis, pulmonary infections, asthma, tuberculosis, genetic disorders (e.g., cystic fibrosis), gas exchange problems, burns, barotraumas and disorders affecting blood supply to the respiratory system. Methods and medicaments of the present invention may also be used to repair, modify, enhance or regenerate the respiratory system following damage. Moreover, methods and compositions of the present invention may be used as a technique to enhance the transplantation/implantation of whole respiratory structures or organs or their parts.

Gastrointestinal Tract and Associated Glands

Examples of diseases, disorders or conditions of the gastrointestinal tract and associated glands that may be treated by the methods and medicaments of the present invention include disorders, damage and age related changes of both the gastrointestinal tract and the large accessory glands (liver and pancreas), salivary glands, mouth, teeth, oesophagus, stomach, duodenum, jejunum, ileum, ascending colon, transverse colon, descending colon, sigmoid colon, rectum and anal canal and enteric nervous system of the canal. In specific embodiments, these disorders, damage and age related changes include dental caries, periodontal disease, deglutition problems, ulcers, enzymatic disturbances/deficiencies, motility problems, paralysis, dysfunction of absorption or absorptive surfaces, diverticulosis, inflammatory bowel problems, hepatitis, cirrhosis and portal hypertension. Methods and medicaments of the present invention may also be used to repair, modify, enhance or regenerate the gastrointestinal tract following damage, or be used as a technique to enhance any of these processes following surgery, such as resection of the stomach, ileostomy and reconstructive surgery (e.g. ileoanal juncture). Examples of this latter embodiment include reconstructive surgery involving specific anatomical structures of the mouth, such as labia, vestibule, oral cavity proper, red margin, labial frenulum, hard palate palatine bones, soft palate, uvula, tongue, intrinsic muscles of the tongue and extrinsic muscles of the tongue.

Integumentary System

Examples of diseases, disorders or conditions of the integumentary system that may be treated by the methods and medicaments of the present invention include disorders, damage and age related changes of the skin and integumentary system, such as age related decline in thickness or function, disorders of sweat gland and sebaceous glands, piloerectile dysfunction, follicular problems, hair loss, epidermal disease, diseases of the dermis or hypodermis, burns, ulcers, sores and infections. Methods and products of the present invention may also be used to enhance, regenerate or repair skin structures or functions, for example in plastic reconstruction, cosmetic repair, tattoo removal, wound healing, modulation of wrinkles and in the treatment of striae, seborrhoea, rosacea, port wine stains, skin colour and the improvement of blood supply to the skin. Moreover, methods and products of the present invention may be used to enhance skin grafts, surgical reconstruction, cosmetic surgical procedures, wound healing and cosmetic appearance.

Musculoskeletal System

Examples of diseases, disorders or conditions of the musculoskeletal system that may be treated by the methods and products of the present invention include disease, damage and age related changes of the musculoskeletal system. In some embodiment, these may be in components of the axial skeleton, including the skull, cranium, face, skull associated bones, auditory ossicles, hyoid bone, sternum, ribs, vertebrae, sacrum and coccyx. In other embodiments they may be in components of the appendicular skeleton, including the clavicle, scapula, humerus, radius, ulna, carpal bones, metacarpal bones, phalanges (proximal, middle, distal), pelvic girdle, femur, patella, tibia, fibula, tarsal bones and metatarsal bones. Methods and compositions of the present invention may also be used to correct problems associated with ossification and osteogenesis, such as intrametnbranous ossification, endochondral ossification, bone remodeling and repair, osteoporosis, osteomalacia, rickets, pagets disease, rheumatism and arthritis. Moreover, methods and products of the present invention may be used to treat disease, damage and age related changes of the skeletal muscle, elastic cartilages, fibrocartilages, long bones, short bones, flat bones and irregular bones.

Other Systems of the Body

Diseases, disorders or conditions of other systems of the body may be treated by the methods and products of the present invention. For example, the present invention may be used to enhance function or treat disease, damage and age related changes in other systems of the body, including special senses, endocrine system, lymphatic system, urinary system, reproductive system and alterations in metabolism and energetics.

Treatment of General Age-Related Degeneration

Methods and compositions of the present invention may be used to treat, ameliorate, reduce or compensate for general age-related degeneration. Similarly, methods and compositions of the present invention can be used to retain youthful functions of the body. Moreover, methods and products of the present invention may be used to treat specific age related system dysfunction, such as cognitive impairment, hearing loss, loss of visual activity, endocrine imbalances, skeletal changes and loss of reproductive function.

Cosmetic Use

In some embodiments, methods and compositions of the present invention may be used to prevent or reduce scars at a site of injury or infection. For example, microvesicles or microRNAs may be employed to regenerate tissue that would otherwise scar or necrotize, including hepatic tissue in the treatment of hepatic fibrosis and/or cirrhosis, facial epidermal tissue to treat acne, and cardiac tissue in the treatment of ischemic infarction.

In some embodiments, methods and compositions (e.g., microvesicles and/or microRNAs) according to the present invention may be used to enhance breast augmentation following mastectomy,

### IV. Pharmaceutical Compositions

In certain embodiments, the present invention provides pharmaceutical compositions comprising a therapeutically effective amount of microvesicles or microRNAs for the treatment of various diseases, disorders or conditions described herein. In some embodiments, the present invention provides pharmaceutical compositions comprising a therapeutically effective amount of microvesicles or microRNAs for the treatment of diabetes mellitus, myocardial infarct, kidney disease, wound healing, fistulas generation or regeneration, neural regeneration, breast augmentation following mastectomy, and/or conditions associated with a cosmetic surgical procedure.

In certain embodiments, the present invention provides pharmaceutical compositions comprising one or more microRNAs having a sequence at least 70% (e.g., 75%, 80%, 85%, 90%, 95%, 96%, 97%, 98%, 99%) identical to any of microRNAs identified in Table 1 and Tables 7-13 (e.g., SEQ ID NOS. 1-29) and a pharmaceutically acceptable carrier. As used herein, the term “pharmaceutically acceptable carrier” includes carriers that are approved by a regulatory agency of government or listed in the United States Pharmocopeia, the European Pharmocopeia, the United Kingdom Pharmocopeia, or other generally recognized pharmocopeia for use in animals, and in particular humans. As used herein, the term “carrier” refers to a diluent, adjuvant, excipient, or vehicle with which a therapeutic agent (e.g., microvesicles and/or microRNAs) is administered.

Provided compositions may also contain minor amounts of wetting agents, emulsifying agents, and/or pH buffering agents. Provided compositions can take any of a variety of solid, liquid, or gel forms, including solutions, suspensions, emulsions, tablets, pills, capsules, powders, sustained-release formulations, and the like. Non-limiting examples of suitable pharmaceutical carriers are described in “Remington's Pharmaceutical Sciences” by E. W. Martin. Compositions will generally contain a therapeutically effective amount of microvesicles and/or microRNAs, optionally in purified form, together with a suitable amount of carrier so as to provide the form for proper administration to the patient.

Formulations are typically adapted to suit the mode of administration. For example, compositions for intravenous administration may be formulated as solutions in sterile isotonic aqueous buffer. Such compositions may also include a solubilizing agent and/or a local anesthetic such as lidocaine (also known as lignocaine, xylocaine, or xylocard) to ease pain at the site of injection.

As further example, compositions for topical and/or local use may be formulated, for example, as a lotion or cream comprising a liquid or semi-solid oil-in-water or water-in-oil emulsion and ointments. Such compositions may also comprise a preservative.

Compositions for delivery to the eye include may be formulated, for example, as eye drops that comprise the active ingredient in aqueous or oily solution and eye ointments that may be manufactured in sterile form. Compositions for delivery to the nose may be formulated, for example, as aerosols or sprays, coarse powders to be rapidly inhaled, or nose drops that comprise the active ingredient (e.g., microvesicles and/or microRNAs) in aqueous or oily solution. Compositions for local delivery to the buccal cavity may be formulated, for example, as lozenges that comprise the active ingredient in a mass generally formed of sugar and gum arabic or tragacanth, and pastilles that comprise the active ingredient in an inert mass (for example of gelatine and glycerine or sugar and gum arabic). Flavoring ingredients may be added to lozenges or pastilles.

Aerosol and spray formulations may comprise, for example, a suitable pharmaceutically acceptable solvent (such as ethanol and water) or a mixture of such solvents. In some embodiments, such formulations comprise other pharmaceutical adjuncts (such as non-ionic or anionic surface-active agents, emulsifiers, and stabilizers) and/or active ingredients of other kinds. Aerosol and spray formulations may be mixed with a propellant gas, such as an inert gas under elevated pressure or with a volatile liquid (e.g., a liquid that boils under normal atmospheric pressure below customary room temperature, for example from −30 to +10° C.).

Routes of Administration and Dosage Regimens

In methods of treatment or of inducing tissue repair, remodeling or differentiation in vivo of the present invention, microvesicles, miRNAs, or a pharmaceutical composition thereof, will generally be administered in such amounts and for such a time as is necessary or sufficient to achieve at least one desired result. For example, miRNAs can be administered in such amounts and for such a time that it amelioriates one or more symptoms of a disease, disorder, or condition; prolongs the survival time of patients; or otherwise yields clinical benefits.

A dosing regimen according to the present invention may consist of a single dose or a plurality of doses over a period of time. Administration may be, e.g., one or multiple times daily, weekly (or at some other multiple day interval), biweekly, monthly, or on an intermittent schedule. Typically an effective amount is administered. The effective amount of microvesicles, microRNAs, or a pharmaceutical composition thereof, will vary from subject to subject and will depend on several factors (see below).

Microvesicles, microRNAs, or pharmaceutical compositions thereof, may be administered using any administration route effective for achieving the desired therapeutic effect. Both systemic and local routes of administration may be used in accordance with methods of the invention. Suitable routes of administration include, but are not limited to, intravenous, intra- arterial, intramuscular, subcutaneous, cutaneous (e.g., topical), intradermal, intracranial, intrathecal, intrapleural, intra-orbital, intranasal, oral, intra-alimentary (e.g., via suppository), colorectal (e.g., via suppository), and intra-cerebrospinal.

Depending on the route of administration, effective doses may be calculated according to, e.g., the body weight and/or body surface area of the patient, the extent of damaged or diseased tissue, etc, Optimization of the appropriate dosages can readily be made by one skilled in the art, e.g., by a clinician. The final dosage regimen is typically determined by the attending physician, considering various factors that might modify the action of the microvesicles, mi RNAs, or pharmaceutical compositions thereof (collectively referred herein as “drug”), e.g., the drug's specific activity, the severity of tissue damage and the responsiveness of the patient, the age, condition, body weight, sex and diet of the patient, the severity of any present infection, time of administration, the use (or not) of other therapies, and other clinical factors.

Typical dosages comprise 1 fg/kg body weight to 1 mg/kg body weight. In some embodiments, dosages range from 100 pg/kg body weight to 1 mg/kg body weight, 10 pg/kg body weight to 1 mg/kg body weight, 1 pg/kg body weight to 1 mg/kg body weight, 100 ng/kg body weight to 1 mg/kg body weight, 10 ng/kg body weight to 1 mg/kg body weight, or 1 ng/kg body weight to 1 mg/kg body weight.

### EXEMPLIFICATION

### Example 1

### Morphological Examination of Pancreas-Derived Pathfinder Cells (PDPC) and Identification of Microvesicles (MVs)

In the present Example, morphological studies of rat pancreas-derived pathfinder cells (PDPC) were conducted by scanning electron microscopy (EM). Scanning EM images revealed protrusions from surfaces of PDPCs that are provisionally identified as nascent microvesicles (MVs).

Pathfinder cells were isolated from rat pancreas cultured as previously described. (See, e.g., International Patent Publication No. WO2006/120476 A1, the entire contents of which are herein incorporated by reference.) These rat PDPCs were grown in medium containing fetal bovine serum (FBS) that was depleted of bovine microvesicles.

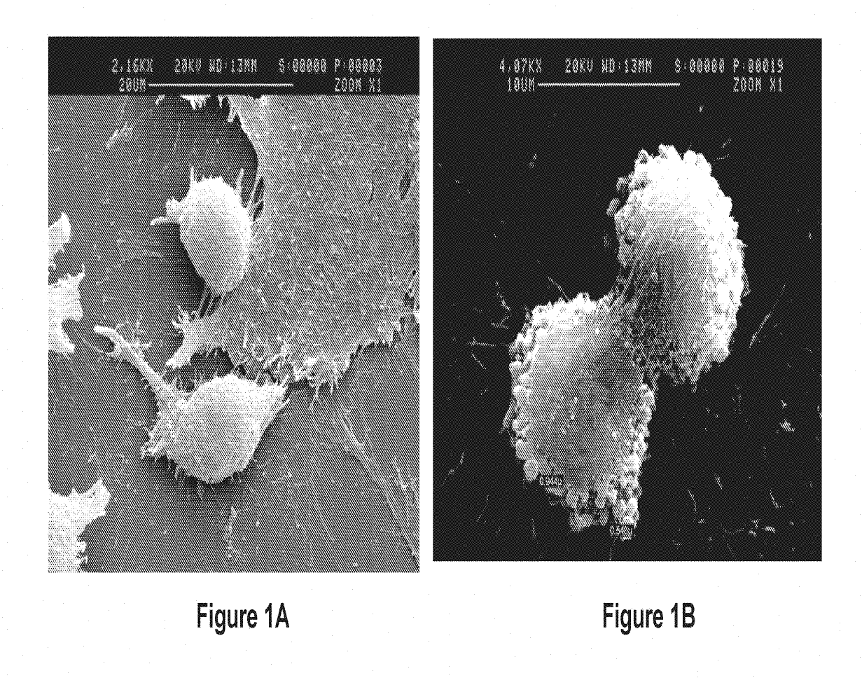

Pictures of a subconfluent culture of rat PDPCs were taken by a scanning electron microscope. FIG. 1A shows a representative picture, showing PDPCs of both the fibroblastoid and small round cell types. As can be seen in FIG. 1A, both cell types have very great numbers of thin projections and interconnect with other cells at multiple points in a complex manner. Furthermore, these cells produce large numbers of small spheres on their surfaces, which are identified as nascent microvesicles (FIG. 1B).

The flat cell type depicted in FIG. 1A is approximately 15-20 μm in diameter, and is the predominant cell type in cultures that were studied. The other cell type is approximately 3-5 μm in size, spherical in morphology, and is commonly found adjoined to an identical cell type. Without wishing to be bound by any particular theory, these spherical cells may be derived from a cell that has recently undergone cell division.

Protrusions of varying length can be seen radiating from the edges of the flatter, larger cell type in particular. Putative microvesicles (MVs) were clearly observed at the ends of these cell protrusions. In some cases, the MVs were not actually attached to the cells but were still within the vicinity of cells and of attached MVs. MVs were also clearly seen close to and surrounding the membrane of the small cell type (FIG. 1B). Clusters of MVs were observed in some areas, typically at the end of a cell protrusion. Identified MVs typically had a size range of 300-600 nm in diameter.

### Example 2

### Analysis of miRNA Expression In Rat PDPCs and In MVs Isolated from Rat PDPCs

Results from Example 1 may shed light into the mechanism of PC action on other cells and tissues. To further investigate the mechanism of PC action, microvesicles obtained from PDPCs were studied in further detail.

In the present Example, MVs were purified from supernatants of rat PDPC cultures in medium with serum depleted of bovine microvesicles using a differential centrifugation protocol. RNA was prepared from both MVs and PDPCs using standard procedures. RNA samples were reverse-transcribed (RT) and amplified in a quantitative PCR assay in order to analyze expression of miRNAs.

### Materials and Methods

RNA extraction. RNA from cells and microvesicles (MVs) was extracted using TRI: Reagent (Sigma), with the following modifications to the manufacturer's protocol. After addition of 1/5th volume chloroform to the TRI Reagent, samples were spun at 6° C. for 15 minutes at 16,000×g. Aqueous phases were then subject to an extraction by phenol:choloform:isoamyl alcohol (pH 6.6; Ambion) at 10° C. for 10 minutes at 16,000×g. Aqueous phases were precipitated for a maximum of 2 hours at −20° C. After centrifugation at 6° C. for 30 minutes at 16,000×g, the resultant RNA was washed in 95% ice-cold ethanol. The RNA was then resuspended in DEPC-water and quantified using a NanoDrop 1000 spectrophotometer.

miRNA analysis. RNA from cells and MVs was analysed for expression of microRNAs (miRNAs) using Applied Biosystem's Taqman Low Density Arrays (TLDA) cards. For rat PDPCs, Taqman Rodent MicroRNA Arrays A and B were used in combination with MegaPlex RT Rodent Pool A and Pool B primers. MV RNA was analysed by Array A according to manufacturer's protocol; analysis with Array B is ongoing,

Results

miRNA distributions in cells and MVs were compared. Table 1 depicts results from analysis of 373 miRNAs from rat PDPC MV RNA preparations. As shown in Table 2, of the 373 miRNAs analyzed, 20 were found to be present only in MVs, with undetectable levels in the cell RNA population. 23 further miRNAs were also only detectable in N/IV's, but these miRNAs were expressed at low levels. Seventeen miRNAs were detected in cell RNA but could not be detected in MV RNA.

TABLE 2
Comparison of miRNA distribution between rat PDPC RNA
preparations and rat PDPC-derived MV RNA preparations

| Distribution pattern | Number of miRNAs |
|---|---|
| miRNAs in MVs but not cells | 52 (23 in low amounts) |
| | Updated: 38 (28 in low amounts; |
| | 16 in high amounts - see Table 2.) |
| miRNAs at higher concentrations | 42 (13 more than 20× higher) |
| in MVs compared to cells | |
| miRNAs at the same | 43 |
| concentration in MVs compared to | |
| cells | |
| miRNAs at lower concentrations | 88 |
| in MVs compared to cells | |
| miRNAs absent in MVs but | 17 |
| present in cells | |
| miRNAs tested but not detected in | 131 |
| either cells or MVs | |

Further work refined the number of miRNAs present in MVs but not in PDPCs to 38, of which 22 miRNAs were present at low levels, Table 3 shows an updated list of miRNAS found in MVs but not cells. Exemplary sequences for these miRNAs are shown in Table 1 and in Appendix 1. Without wishing to be bound by any particular theory, the presence of some miRNAs in MVs but not in cells suggest that these MVs were likely produced in the MVs.

TABLE 3
miRNAs found in rat PDPC MVs but not cells
miRNAs unique to MVs
(see Table 1 and Appendix 1 for exemplary sequences)

| Higher concentrations | Lower concentrations |
|---|---|
| (Ct less than 32) | (Ct more than 32) |
| miR122, miR127, miR133b, | miR136, miR202, miR206, miR224, |
| miR323, miR346, miR433, | miR327, miR347, miR369, miR370, |
| miR451, miR466h, miR467c, | miR375, miR376b, miR381, miR434, |
| miR467e, miR468, miR491, | miR452, miR465a, miR465b, miR470, |
| miR495, miR546, miR666, | miR487b, miR543, miR547, miR590, |
| miR680. | miR741, miR881. |
| (16 in total) | (22 in total) |

Table 4 lists the miRNAs that were found in cells but not in microvesicles. Sequences shown are sequences from Rattus norvegicus. Sequences of corresponding miRNAs from other species including Homo sapiens and Mus musculus are also known in the art; e.g., see http://diana.cslab.ece.ntua.gr/mirgen/.

TABLE 4
miRNAs found in rat PDPCs but not MVs

| | Exemplary Sequence(s) |
|---|---|
| miRNA | (5′ to 3′) |
| miR7b | UGGAAGACUUGUGAUUUUGUUGU |
| | (SEQ ID NO: 73) |
| miR17-3p | ACUGCAGUGAAGGCACUUGUGG |
| | (SEQ ID NO: 74) |
| miR32 | UAUUGCACAUUACUAAGUUGCA |
| | (SEQ ID NO: 75) |
| miR34c | AGGCAGUGUAGUUAGCUGAUUGC |
| | (SEQ ID NO: 76) |
| | AAUCACUAACCACACAGCCAGG |
| | (SEQ ID NO: 77) |
| | (variant) |
| miR129-5p | CUUUUUGCGGUCUGGGCUUGC |
| | (SEQ ID NO: 78) |
| miR190 | UGAUAUGUUUGAUAUAUUAUGU |
| | (SEQ ID NO: 79) |
| miR203 | GUGAAAUGUUUAGGACCACUAG |
| | (SEQ ID NO: 80) |
| miR376c | AACAUAGAGGAAAUUUCACGU |
| | (SEQ ID NO: 81) |
| miR381 | UAUACAAGGGCAAGCUCUC |
| | (SEQ ID NO: 82) |
| miR384-3p | AUUCCUAGAAAUUGUUCACAAU |
| | (SEQ ID NO: 83) |
| miR455 | UAUGUGCCUUUGGACUACAUCG |
| | (SEQ ID NO: 84) |
| miR499 | UUAAGACUUGCAGUGAUGUUU |
| | (SEQ ID NO: 85) |
| miR505 | GUCAACACUUGCUGGUUUCC |
| | (SEQ ID NO: 86) |
| miR582-5p | UACAGUUGUUCAACCAGUUACU |
| | (SEQ ID NO: 87) |
| miR615-3p | UCCGAGCCUGGGUCUCCCUCUU |
| | (SEQ ID NO: 88) |
| miR615-5p | GGGGGUCCCCGGUGCUCGGAUC |
| | (SEQ ID NO: 89) |

These results demonstrate that MVs do not contain a merely random sample of cytoplasmic or endosomal content. Without wishing to be bound by any particular theory, miRNAs that are specifically present in MVs may be candidates for intercellular regulators. These MV-specific miRNAs may be individually validated using assays such as those described in Examples 3 and 4.

### Example 3

### Assays for Characterizing Effects of MVs or miRNAs on Cell Growth

The present Example demonstrates the effects of MVs on growth of rat PDPCs.

An XCELLINGENCE™ machine was used to measure cell growth in rat PDPC cultures that were depleted of bovine MVs, or depleted of MVs and then had rat PDPC MVs added back.

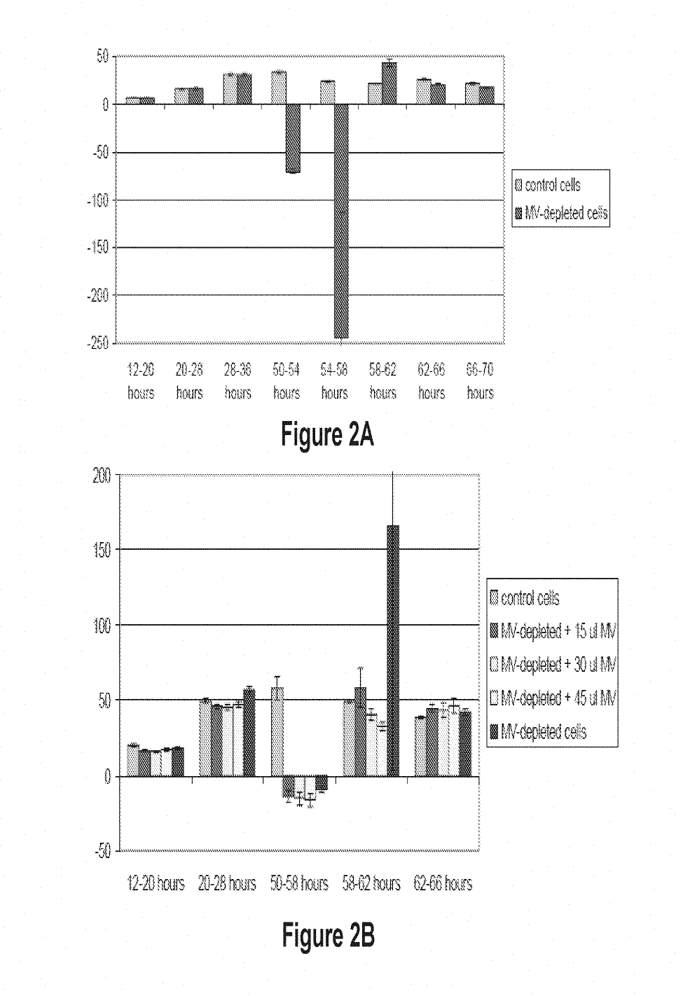

Rat PDPCs were cultured in medium containing bovine serum, and then at 43 hours were switched to bovine MV-depleted medium. Depleting MVs resulted in a decrease in cell proliferation, with a doubling time slowing to 31 hours (FIG. 2A). A negative effect on doubling time was seen, with a later recovery.

In a separate set of experiments, cultures were MV-depleted at 48 hours, and then exogenous MVs are added 10 hours later. A dose-dependent recovery of rat PDPC doubling time (i.e., increase in cell proliferation) was observed after addition of rat PDPC-derived. MVs (FIG. 2B). The increase in cell proliferation persisted for 48 hours and then faded. The rapid recovery of doubling time of cells receiving exogenous MV occurred well in advance of the normal recovery time.

These results not only show that MVs can increase cell proliferation; they also provide a possible assay for characterize effects of individual miRNAs on PDPC growth rate. Similar assays may also be developed for PC effects on target cell types.

The effects of MVs on growth rates of other PCs may be tested similarly. For example, human kidney-derived Pathfinder cells (KDPCs) and lymph node-derived pathfinder cells (LNDPCs) may be used instead of PDPCs.

### Example 4

### In Vitro Cell Damage Assay

This Example demonstrates that an in vitro assay has been successfully developed to assess the effects of MVs or miRNAs on stimulate wound repair or recovery from cell damage.

Fibroblasts are grown to confluence in wells of an XCELLIGENCE™ machine (Roche Applied Science) for use as target cells. Cultures are then scored with a pipette tip to mimic a wound. Cultures are grown in the presence of (1) PCs of various tissue origins; (2) MVs derived from PCs; (3) specific miRNAs analyzed, for example, as described in Example 2; or (4) media without any of the above, as a negative control.

Regrowth of cells across the area of damage is read by the XCELLIGENCE™ machine, which gives a quantitative readout. The effects of PCs, MVs, and particular miRNAs on wound repair may be determined by regrowth rates from the various cultures.

### Example 5

### Production of MVs From Cells Cultured In Low Oxygen Conditions

This Example is designed to show that MV production in PC cells and/or the RNA expression profiles may be optimized by varying certain cell culture conditions. It is postulated that growing cells in hypoxic conditions during culture may reduce secretions of cytokines, which could extend lifespan of cells producing MVs, thereby increasing MV production,

In the present Example, PCs of various cell types are grown in conditions of low oxygen (less than 5% O₂); cultures are also grown in conditions of normal (e.g., about 5% O₂) oxygen to be used as controls. MV production may be quantitated using standard methods or adaptations of known methods, such as, e.g., electron microscopy, FACS, measurement of MV weight and calculation based on known number/weight ratios, etc.

For example, to examine possible effects of low oxygen on RNA content of MVs, MVs are isolated from cultures as described in Example 2. RNA preparations are made from MVs and quantified and amounts are compared between the two groups (low oxygen vs. normal oxygen).

### Example 6

### Isolation and Enrichment of ATVs From Conditioned Media

This Example describes isolation and enrichment of MVs from conditioned media. PCs of various cell types are isolated and cultured as previously described. (See, e.g., International Patent Publication WO2006/120476A1). PCs are expanded to near confluence (sub-confluence) in tissue culture flasks in media free of serum. (Bovine microvesicle-depleted media may also be used.) Media from sub-confluent cultures (“conditioned media”) are collected and analyzed immediately or frozen for further analysis. Conditioned media may be analyzed for MV production by methods known in the art, such as those mentioned in Example 5. MVs may be harvested from conditional media using standard methods. RNA is extracted from conditioned media and total RNA content and amount of specific miRNAs associated with MVs are analyzed.

### Example 7

### Culture of PCs on Nonwoven Substrates to Increase MV Production In Conditioned Media

This Example describes a modified culture method that may increase MV production in conditioned media. PCs are grown on nonwoven fabrics of various compositions and microvesicle production in conditioned culture media is assessed.

Circular substrates of one centimeter in diameter are made from nonwoven fabrics of various compositions:

(1) a fabric comprising fibers of 90/10 poly(glycolide-co-lactide) (PGA/PLA) sold under the tradename VICRYL™ (Ethicon, Inc., Somerville, N.J.);

(2) a fabric comprising fibers of 95/5 poly(lactide-co-glycolide) (PLA/PGA) sold under the tradename 95/5 PLA/PGA™; and

(3) a fabric comprising 50% (90/10 PGA/PLA) fibers and 50% PDO fibers.

Fabrics used in this Example are of 1 mm or 1.5 mm thickness and density ranged from about 60 to about 300 mg/mL.

Fabric substrates are placed in low-cluster 24-well plates and sterilized by soaking in 100% ethanol for four hours. Substrates are then washed with phosphate-buffered saline (PBS) and placed in medium containing fetal bovine serum (FBS) that was depleted of bovine microvesicles.

PCs of various tissue origins are seeded onto the substrates within the wells. A 24-well tissue culture plate without substrates is seeded with PCs as a control. Cell-seeded substrates and control wells are cultured until cultures reach sub-confluence.

Media from sub-confluent cultures (“conditioned media”) is collected from wells and analyzed for MV production, e.g., as described in Example 5. MVs may be harvested from conditioned media using standard methods.

### Example 8

### RNA Expression Profiling of Rat PDPCs

In the present Example, RNA expression profiling was performed on rat PDPCs. PDPCs were cultured and RNA extracted as described in Example 2. Table 5 shows miRNAs that were found to be expressed in PDPCs that may be useful for therapeutic applications described herein, miRNAs that were expressed abundantly are shown in bold. Sequences of these miRNAs can be found in Appendix 1.

TABLE 5
miRNAs expressed in PDPCs
miRNAs

let-7 a*, let-7c-1*, let-7g*
miR-7a*, −9*, 15a*, −15b*, −16*, −17*, −18a*, −21*, −22*, −24-1*,
24-2*, −26b*, −27a*, −27b*, −28*, −29a*, −29b*, −29c*, −30a*, −30e*,
−31*, −33*, −34c*, −93*, −99b*, rno-miR-7a*, −20a*, −20b-5p, −28*,
−30d*, −99a*
miR-101b, −106b*, −125b*, −135a*, −149, −181a-1*, −191*, −193*,
−199b*, rno-miR-125b*, −148b-5p
miR-200a*, −200b*, −206, −214*, −218-1*, −218-2*
miR-322*, −326, −374, −378, −378*, rno-miR-352
miR-425*, −455*, −467a*, −467b*, −470*, −499c
miR-503*, −592
miR-674*, −678, −690, −699, rno-miR-664
miR-709, −720, −721, −744*, −760, −763, rno-miR-743a
miR-872*, −877, −877*

### Example 10

### Microvesicle (MV) Purification

In the present Example, MVs were purified from supernatants of rat PC cultures grown under serum replete or serum starvation conditions using a differential centrifugation protocol according to the schematic in FIG. 3 or a commercially available exosome precipitation kit (Exo-Quick™ Exosome Precipitation, System Biosciences, Mountain View, Calif.). Control MVs from rat mesenchymal stem cells (MSC) grown in serum replete or serum starvation conditions were also purified.

Briefly, for purification using differential centrifugation, 10 mls culture medium was centrifuged at 1000×g for 10 minutes to remove cellular debris. The sample was further centrifuged at 16,0000×g for 90 minutes at 4° C. Pellet (P1) and supernatant (S1) fractions were separated and the pellet fraction was washed with 10 mls of PBS and centrifuged at 16,000×g for 90 minutes at 4° C. The resulting pellet fraction, P2 was resuspended in 0.2 ml buffer. The S1 supernatant fraction was centrifuged at 120,000×g for 120 minutes at 4° C. and the resulting pellet P3 was washed with 5 ml s of PBS and centrifuged at 120,000×g for 120 minutes at 4° C. The resulting pellet fraction, P4 was resuspended in 0.2 ml buffer.

For purification of MVs using Exo-Quick™ Exosome Precipitation (System Biosciences, Mountain View, Calif.), 1 ml of culture medium was treated with Exo-Quick reagent according to the manufacturer instructions. MV pellets were recovered and resuspended in buffer.

Total protein and total RNA were quantitated for fractions obtained by each purification method (differential centrifugation and precipitation) using standard methods. Table 6 shows exemplary total protein and total RNA amounts obtained in each fraction for the purification methods tested.

TABLE 6
Total protein and total RNA from MV purification.

| | Total protein | | | | Total RNA | | | |
|---|---|---|---|---|---|---|---|---|
| Differential | (per fraction) | | | | (per fraction) | | | |
| Centrifugation | MV | | | | MV | | | |
| 10 ml of | fraction | | Exosomes | | fraction | | Exosomes | |
| media used | (P2) | | (P4) | | (P2) | | (P4) | |
| Rat MSCs | 17.4 | μg | 108 | μg | 123 | ng | 431 | ng |
| control | | | | | | | | |
| Rat MSCs | 17.0 | ug | 139 | μg | 216 | ng | 315 | ng |
| serum free | | | | | | | | |
| condition | | | | | | | | |
| (24 h) | | | | | | | | |
| Rat PCs | 9 | μg | 78.2 | μg | 156 | ng | 594 | ng |
| control | | | | | | | | |
| Rat PCs | 8.6 | μg | 69.5 | μg | 466 | ng | 349 | ng |

| serum free | | | | | | |
|---|---|---|---|---|---|---|
| conditions | | | | | | |
| (24 h) | | | | | | |
| | Total protein | | | Total RNA | | |
| Exo-Quick ™ | (per fraction) | | | (per fraction) | | |
| 1 ml of media | MV | | | MV | | |
| used | fraction | Exosomes | | fraction | Exosomes | |
| Rat MSCs | — | 200 | μg | — | 500 | ng |
| control | | | | | | |
| Rat MSCs | — | 250 | μg | — | 389 | ng |
| serum free | | | | | | |

condition
(24 h)

### Example 11

### RNA Expression Profiling of MVs From Serum-Starved PCs

In the present Example, MVs were purified from supernatants of rat or human PC cultures grown under serum starvation conditions for about 24 hours using a differential centrifugation protocol (described in Example 10). RNA was prepared from PCs and MVs as described in Example 2.

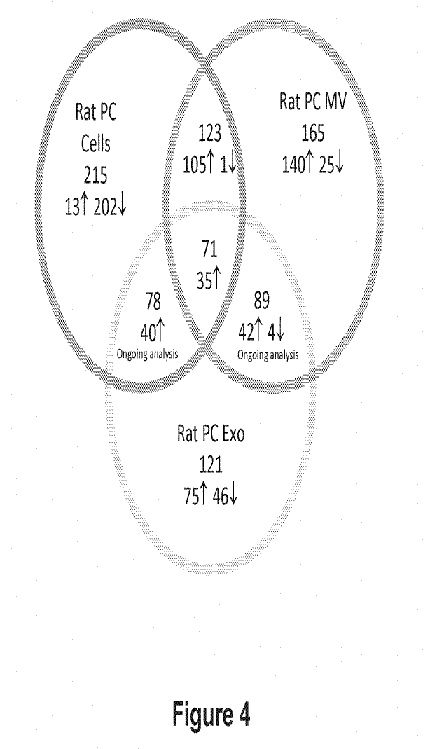

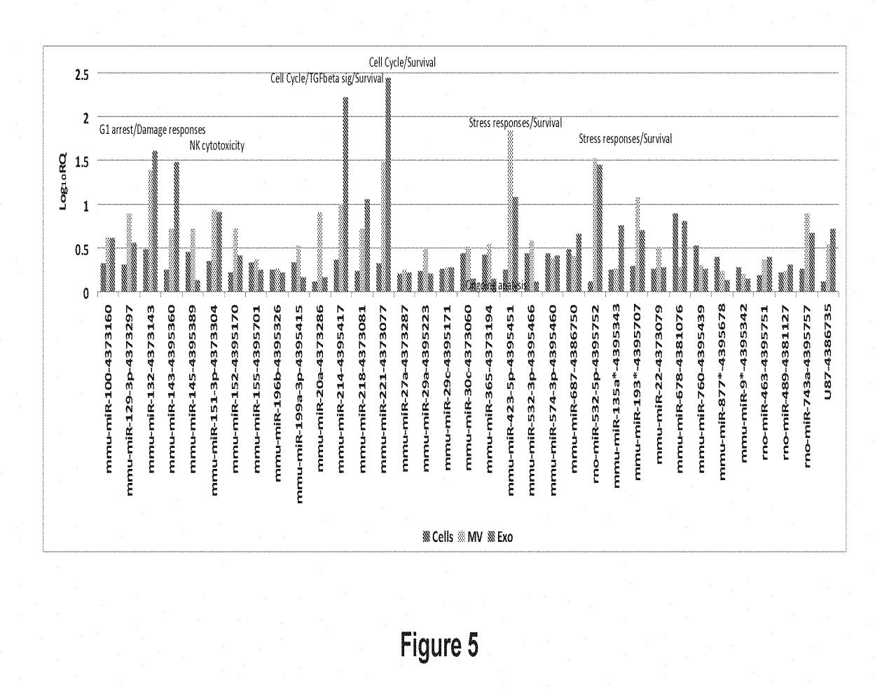

microRNA expression profiles for rat PCs, MV fractions, and exosome fractions were determined and compared. As shown in FIG. 4, microRNA whose expression was altered by growth under serum starvation conditions for 24 hours as compared with growth under serum replete conditions was determined and overlapping microRNA sequences among rat PC's, MV fractions and exosome fractions were identified. As can be seen in FIG. 4, there were 35 miRNAs in common to all samples which had increased expression in response to serum starvation. FIG. 5 shows an exemplary graph comparison of miRNA expression profiles for rat PCs, MV fractions, and exosome fractions. As can be seen in FIG. 5, microRNAs whose expression was increased in response to serum starvation may play roles in various cellular functions, including cell cycle, damage responses, stress responses, cell survival, and immune signaling.

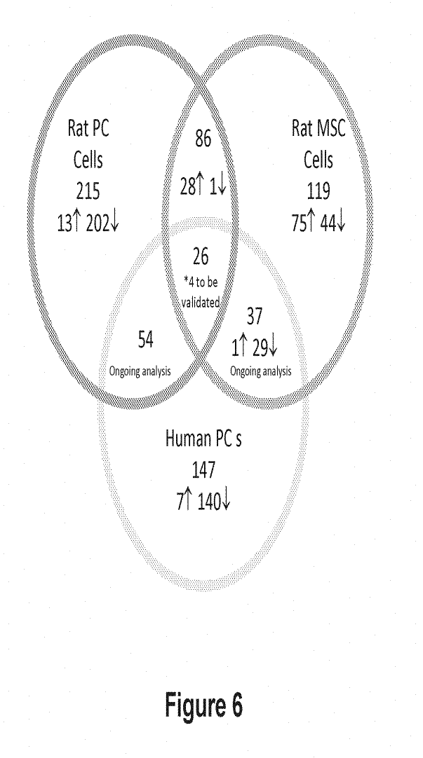

microRNA expression profiles for rat PCs, rat MSC, and human PC were determined and compared. As shown in FIG. 6, microRNA. Whose expression was altered by growth under serum starvation conditions for 24 hours as compared with growth under serum replete conditions were determined and overlapping microRNA sequences among rat PCs, rat MSC, and human PCs were identified. As can be seen in FIG. 6, there were 26 miRNAs in common to all samples which had increased expression in response to serum starvation.

As described above, miRNAs in MVs obtained from rat PC cells grown under serum starvation conditions were identified. Table 7 depicts results from analysis of miRNAs from MVs obtained from rat PC RNA preparations.

TABLE 7
Exemplary miRNA sequences in MVs from serum
starved rat PCs

| miRNA in | | |
|---|---|---|
| MVs from | Exemplary Sequence(s) | Alternative |
| Rat PCs | (5′ to 3′) | Description |
| mmu4et-7d- | AGAGGUAGUAGGUUGCAUAGUU | MIMAT0000383 |
| 4395394 | (SEQ ID NO: 90) | |
| mmu-miR-106a- | CAAAGUGCUAACAGUGCAGGUAG | MIMAT0000385 |
| 4395589 | (SEQ ID NO: 91) | |
| mmu-miR-106b- | UAAAGUGCUGACAGUGCAGAU | MIMAT0000386 |
| 4373155 | (SEQ ID NO: 92) | |
| mmu-miR-10a- | UACCCUGUAGAUCCGAAUUUGUG | MIMAT0000648 |
| 4373153 | (SEQ ID NO: 93) | |
| mmu-miR-126- | UCGUACCGUGAGUAAUAAUGCG | MIMAT0000138 |
| 3p-4395339 | (SEQ ID NO: 94) | |
| mmu-miR-130a- | CAGUGCAAUGUUAAAAGGGCAU | MIMAT0000141 |
| 4373145 | (SEQ ID NO: 95) | |
| mmu-miR-130b- | CAGUGCAAUGAUGAAAGGGCAU | MIMAT0000387 |
| 4373144 | (SEQ ID NO: 96) | |
| mmu-miR-140- | CAGUGGUUUUACCCUAUGGUAG | MIMAT0000151 |
| 4373374 | (SEQ ID NO: 97) | |
| mmu-miR-142- | UGUAGUGUUUCCUACUUUAUGGA | MIMAT0000155 |
| 3p-4373136 | (SEQ ID NO: 98) | |
| mmu-miR-145- | GUCCAGUUUUCCCAGGAAUCCCU | MIMAT0000157 |
| 4395389 | (SEQ ID NO: 99) | |
| mmu-miR-146a- | UGAGAACUGAAUUCCAUGGGUU | MIMAT0000158 |
| 4373132 | (SEQ ID NO: 100) | |
| mmu-miR-146b- | UGAGAACUGAAUUCCAUAGGUU | MIMAT0003475 |
| 4373178 | (SEQ ID NO: 101) | |
| mmu-miR-148b- | UCAGUGCAUCACAGAACUUUGU | MIMAT0000580 |
| 4373129 | (SEQ ID NO: 102) | |
| mmu-miR-155- | UUAAUGCUAAUUGUGAUAGGGGU | MIMAT0000165 |
| 4395701 | (SEQ ID NO: 103) | |
| mmu-miR-15a- | UAGCAGCACAUAAUGGUUUGUG | MIMAT0000526 |
| 4373123 | (SEQ ID NO: 104) | |
| mmu-miR-15b- | UAGCAGCACAUCAUGGUUUACA | MIMAT0000124 |
| 4373122 | (SEQ ID NO: 105) | |
| mmu-miR-16- | UAGCAGCACGUAAAUAUUGGCG | MIMAT0000527 |
| 4373121 | (SEQ ID NO: 106) | |
| mmu-miR-181a- | AACAUUCAACGCUGUCGGUGAGU | MIMAT0000210 |
| 4373117 | (SEQ ID NO: 107) | |
| mmu-miR-186- | CAAAGAAUUCUCCUUUUGGGCU | MIMAT0000215 |
| 4395396 | (SEQ ID NO: 108) | |
| mmu-miR-188- | CAUCCCUUGCAUGGUGGAGGG | MIMAT0000217 |
| 5p-4395431 | (SEQ ID NO: 109) | |
| mmu-miR-193b- | AACUGGCCCACAAAGUCCCGCU | MIMAT0004859 |
| 4395597 | (SEQ ID NO: 110) | |
| mmu-miR-194- | UGUAACAGCAACUCCAUGUGGA | MIMAT0000224 |
| 4373106 | (SEQ ID NO: 111) | |
| mmu-miR-196b- | UAGGUAGUUUCCUGUUGUUGGG | MIMAT0001081 |
| 4395326 | (SEQ ID NO: 112) | |
| mmu-miR-19a- | UGUGCAAAUCUAUGCAAAACUGA | MIMAT0000651 |
| 4373099 | (SEQ ID NO: 113) | |
| mmu-miR-204- | UUCCCUUUGUCAUCCUAUGCCU | MIMAT0000237 |
| 4373094 | (SEQ ID NO: 114) | |
| mmu-miR-20a- | UAAAGUGCUUAUAGUGCAGGUAG | MIMAT0000529 |
| 4373286 | (SEQ ID NO: 115) | |
| mmu-miR-210- | CUGUGCGUGUGACAGCGGCUGA | MIMAT0000658 |
| 4373089 | (SEQ ID NO: 116) | |
| mmu-miR-21- | UAGCUUAUCAGACUGAUGUUGA | MIMAT0000530 |
| 4373090 | (SEQ ID NO: 117) | |
| mmu-miR-214- | ACAGCAGGCACAGACAGGCAGU | MIMAT0000661 |
| 4395417 | (SEQ ID NO: 118) | |
| mmu-miR-218- | UUGUGCUUGAUCUAACCAUGU | MIMAT0000663 |
| 4373081 | (SEQ ID NO: 119) | |
| mmu-miR-23b- | AUCACAUUGCCAGGGAUUACC | MIMAT0000125 |
| 4373073 | (SEQ ID NO: 120) | |
| mmu-miR-24- | UGGCUCAGUUCAGCAGGAACAG | MIMAT0000219 |
| 4373072 | (SEQ ID NO: 121) | |
| mmu-miR-25- | CAUUGCACUUGUCUCGGUCUGA | MIMAT0000652 |
| 4373071 | (SEQ ID NO: 122) | |
| mmu-miR-26a- | UUCAAGUAAUCCAGGAUAGGCU | MIMAT0000533 |
| 4395166 | (SEQ ID NO: 123) | |
| mmu-miR-26b- | UUCAAGUAAUUCAGGAUAGGU | MIMAT0000534 |
| 4395167 | (SEQ ID NO: 124) | |
| mmu-miR-27b- | UUCACAGUGGCUAAGUUCUGC | MIMAT0000126 |
| 4373068 | (SEQ ID NO: 125) | |
| mmu-miR-296- | AGGGCCCCCCCUCAAUCCUGU | MIMAT0000374 |
| 5p-4373066 | (SEQ ID NO: 126) | |
| mmu-miR-29c- | UAGCACCAUUUGAAAUCGGUUA | MIMAT0000536 |
| 4395171 | (SEQ ID NO: 127) | |
| mmu-miR-301a- | CAGUGCAAUAGUAUUGUCAAAGC | MIMAT0000379 |
| 4373064 | (SEQ ID NO: 128) | |
| mmu-miR-301b- | CAGUGCAAUGGUAUUGUCAAAGC | MIMAT0004186 |
| 4395730 | (SEQ ID NO: 129) | |
| mmu-miR-30a- | UGUAAACAUCCUCGACUGGAAG | MIMAT0000128 |
| 4373061 | (SEQ ID NO: 130) | |
| mmu-miR-30c- | UGUAAACAUCCUACACUCUCAGC | MIMAT0000514 |
| 4373060 | (SEQ ID NO: 131) | |
| mmu-miR-30d- | UGUAAACAUCCCCGACUGGAAG | MIMAT0000515 |
| 4373059 | (SEQ ID NO: 132) | |
| mmu-miR-30e- | UGUAAACAUCCUUGACUGGAAG | MIMAT0000248 |
| 4395334 | (SEQ ID NO: 133) | |
| mmu-miR-320- | AAAAGCUGGGUUGAGAGGGCGA | MIMAT0000666 |
| 4395388 | (SEQ ID NO: 134) | |
| mmu-miR-322- | CAGCAGCAAUUCAUGUUUUGGA | MIMAT0000548 |
| 4378107 | (SEQ ID NO: 135) | |
| mmu-miR-324- | CCACUGCCCCAGGUGCUGCU | MIMAT0000556 |
| 3p-4395639 | (SEQ ID NO: 136) | |
| mmu-miR-328- | CUGGCCCUCUCUGCCCUUCCGU | MIMAT0000565 |
| 4373049 | (SEQ ID NO: 137) | |
| mmu-miR-331- | GCCCCUGGGCCUAUCCUAGAA | MIMAT0000571 |
| 3p-4373046 | (SEQ ID NO: 138) | |
| mmu-miR-335- | UUUUUCAUUAUUGCUCCUGACC | MIMAT0004704 |
| 3p-4395296 | (SEQ ID NO: 139) | |
| mmu-miR-34a- | UGGCAGUGUCUUAGCUGGUUGU | MIMAT0000542 |
| 4395168 | (SEQ ID NO: 140) | |
| mmu-miR-34b- | AAUCACUAACUCCACUGCCAUC | MIMAT0004581 |
| 3p-4395748 | (SEQ ID NO: 141) | |
| mmu-miR-351- | UCCCUGAGGAGCCCUUUGAGCC | MIMAT0000609 |
| 4373345 | UG | |
| | (SEQ ID NO: 142) | |
| mmu-miR-363- | AAUUGCACGGUAUCCAUCUGUA | MIMAT0000708 |
| 4378090 | (SEQ ID NO: 143) | |
| mmu-miR-365- | UAAUGCCCCUAAAAAUCCUUAU | MIMAT0000711 |
| 4373194 | (SEQ ID NO: 144) | |
| mmu-miR-410- | AAUAUAACACAGAUGGCCUGU | MIMAT0001091 |
| 4378093 | (SEQ ID NO: 145) | |
| mmu-miR-434- | UUUGAACCAUCACUCGACUCCU | MIMAT0001422 |
| 3p-4395734 | (SEQ ID NO: 146) | |
| mmu-miR-497- | CAGCAGCACACUGUGGUUUGUA | MIMAT0003453 |
| 4381046 | (SEQ ID NO: 147) | |
| mmu-miR-574- | CACGCUCAUGCACACACCCACA | MIMAT0004894 |
| 3p-4395460 | (SEQ ID NO: 148) | |
| mmu-miR-652- | AAUGGCGCCACUAGGGUUGUG | MIMAT0003711 |
| 4395463 | (SEQ ID NO: 1492) | |
| mmu-miR-667- | UGACACCUGCCACCCAGCCCAAG | MIMAT0003734 |
| 4386769 | (SEQ ID NO: 150) | |
| mmu-miR-743b- | UGUUCAGACUGGUGUCCAUCA | MIMAT0004839 |
| 5p-4395600 | (SEQ ID NO: 151) | |
| mmu-miR-93- | CAAAGUGCUGUUCGUGCAGGUAG | MIMAT0000540 |
| 4373302 | (SEQ ID NO: 152) | |
| mmu-miR-99b- | CACCCGUAGAACCGACCUUGCG | MIMAT0000132 |
| 4373007 | (SEQ ID NO: 153) | |
| rno-miR-196c- | UAGGUAGUUUCGUGUUGUUGGG | MIMAT0005303 |
| 4395750 | (SEQ ID NO: 154) | |
| rno-miR-351- | UCCCUGAGGAGCCCUUUGAGCC | MIMAT0000608 |
| 4395764 | UGA | |
| | (SEQ ID NO: 155) | |
| rno-miR-532- | CAUGCCUUGAGUGUAGGACUGU | MIMAT0005322 |
| 5p-4395752 | (SEQ ID NO: 156) | |
| snoRNA135- | CTAAAATAGCTGGAATTACCGG | Mature |
| 4380912 | CAGATTGGTAGTGGTGAGCCTA | miRNA |
| | TGGTTTTCTGAAG | Control |
| | (SEQ ID NO: 157) | |
| U87- | ACAATGATGACTTATGTTTTT | Mature |
| 4386735 | GCCGTTTACCCAGCTGAGGGT | miRNA |
| | TTCTTTGAAGAGAGAATC | Control |
| | TTAAGACTGAGC | |
| | (SEQ ID NO: 158) | |
| mmu-let-7a*- | CUAUACAAUCUACUGUCUUUCC | MIMAT0004620 |
| 4395608 | (SEQ ID NO: 159) | |
| mmu-miR- | ACAAGUCAGGUUCUUGGGACCU | MIMAT0004529 |
| 125b*-4395638 | (SEQ ID NO: 160) | |
| mmu-miR- | ACUCUUUCCCUGUUGCACUACU | MIMAT0004583 |
| 130b*-4395590 | (SEQ ID NO: 161) | |
| mmu-miR- | UAUAGGGAUUGGAGCCGUGGCG | MIMAT0004531 |
| 135a*-4395343 | (SEQ ID NO: 162) | |
| mmu-miR-136*- | AUCAUCGUCUCAAAUGAGUCUU | MIMAT0004532 |
| 4395642 | (SEQ ID NO: 163) | |
| mmu-miR-138*- | CGGCUACUUCACAACACCAGGG | MIMAT0004668 |
| 4395684 | (SEQ ID NO: 164) | |
| mmu-miR-141*- | CAUCUUCCAGUGCAGUGUUGGA | MIMAT0004533 |
| 4395643 | (SEQ ID NO: 165) | |
| mmu-miR-149- | UCUGGCUCCGUGUCUUCACUCCC | MIMAT0000159 |
| 4395366 | (SEQ ID NO: 166) | |
| mmu-miR-186*- | GCCCUAAGGUGAAUUUUUUGGG | MIMAT0004540 |
| 4395704 | (SEQ ID NO: 167) | |
| mmu-miR-190b- | UGAUAUGUUUGAUAUUGGGUU | MIMAT0004852 |
| 4395374 | (SEQ ID NO: 168) | |
| mmu-miR- | UCGGCAACAAGAAACUGCCUGA | MIMAT0004618 |
| 196a*-4395607 | (SEQ ID NO: 169) | |
| mmu-miR-206- | UGGAAUGUAAGGAAGUGUGUGG | MIMAT0000239 |
| 4373092 | (SEQ ID NO: 170) | |
| mmu-miR-26b*- | CCUGUUCUCCAUUACUUGGCUC | MIMAT0004630 |
| 4395555 | (SEQ ID NO: 171) | |
| mmu-miR-29b*- | GCUGGUUUCAUAUGGUGGUUUA | MIMAT0004523 |
| 4395627 | (SEQ ID NO: 172) | |
| mmu-miR-322*- | AAACAUGAAGCGCUGCAACAC | MIMAT0000549 |
| 4395636 | (SEQ ID NO: 173) | |
| mmu-miR-33*- | CAAUGUUUCCACAGUGCAUCAC | MIMAT0004666 |
| 4395247 | (SEQ ID NO: 174) | |
| mmu-miR-34c*- | AAUCACUAACCACACAGCCAGG | MIMAT0004580 |
| 4395714 | (SEQ ID NO: 175) | |
| mmu-miR-378- | ACUGGACUUGGAGUCAGAAGG | MIMAT0003151 |
| 4395354 | (SEQ ID NO: 176) | |
| mmu-miR-466d- | UAUACAUACACGCACACAUAG | MIMAT0004931 |
| 3p-4395665 | (SEQ ID NO: 177) | |
| mmu-miR- | AUAUACAUACACACACCAACAC | MIMAT0003478 |
| 467b*-4381092 | (SEQ ID NO: 178) | |
| mmu-miR-673- | CUCACAGCUCUGGUCCUUGGAG | MIMAT0003739 |
| 5p-4386772 | (SEQ ID NO: 179) | |
| mmu-miR-674*- | CACAGCUCCCAUCUCAGAACAA | MIMAT0003741 |
| 4386773 | (SEQ ID NO: 180) | |
| mmu-miR-678- | GUCUCGGUGCAAGGACUGGAGG | MIMAT0003452 |
| 4381076 | (SEQ ID NO: 181) | |
| mmu-miR-690- | AAAGGCUAGGCUCACAACCAAA | MIMAT0003469 |
| 4381086 | (SEQ ID NO: 182) | |
| mmu-miR-696- | GCGUGUGCUUGCUGUGGG | MIMAT0003483 |
| 4381051 | (SEQ ID NO: 183) | |
| mmu-miR-697- | AACAUCCUGGUCCUGUGGAGA | MIMAT0003487 |
| 4381054 | (SEQ ID NO: 184) | |
| mmu-miR-709- | GGAGGCAGAGGCAGGAGGA | MIMAT0003499 |
| 4381063 | (SEQ ID NO: 185) | |
| mmu-miR-715- | CUCCGUGCACACCCCCGCGUG | MIMAT0003506 |
| 4381067 | (SEQ ID NO: 186) | |
| mmu-miR-720- | AUCUCGCUGGGGCCUCCA | MIMAT0003484 |
| 4381052 | (SEQ ID NO: 187) | |
| mmu-miR-721- | CAGUGCAUUAAAAGGGGGAA | MIMAT0003515 |
| 4381073 | (SEQ ID NO: 188) | |
| mmu-miR-760- | CGGCUCUGGGUCUGUGGGGA | MIMAT0003898 |
| 4395439 | (SEQ ID NO: 189) | |
| mmu-miR-801- | GAUUGCUGUGCGUGCGGAAUC | |
| 4395562 | GAC | |
| | (SEQ ID NO: 190) | |
| mmu-miR-805- | GAAUUGAUCAGGACAUAGGG | MIMAT0004211 |
| 4395577 | (SEQ ID NO: 191) | |
| mmu-miR-872*- | UGAAGUAUUGCAGUAGCCUCCU | MIMAT0004935 |
| 4395672 | (SEQ ID NO: 192) | |
| mmu-miR-875- | UAUACCUCAGUUUUAUCAGGUG | MIMAT0004937 |
| 5p-4395314 | (SEQ ID NO: 193) | |
| mmu-miR-877*- | UGUCCUCUUCUCCCUCCUCCCA | MIMAT0004862 |
| 4395678 | (SEQ ID NO: 194) | |
| mmu-miR-877- | GUAGAGGAGAUGGCGCAGGG | MIMAT0004861 |
| 4395402 | (SEQ ID NO: 195) | |
| mmu-miR-878- | GCAUGACACCACACUGGGUAGA | MIMAT0004933 |
| 3p-4395671 | (SEQ ID NO: 196) | |
| mmu-miR-93*- | ACUGCUGAGCUAGCACUUCCCG | MIMAT0004636 |
| 4395250 | (SEQ ID NO: 197) | |
| mmu-miR-99b*- | CAAGCUCGUGUCUGUGGGUCCG | MIMAT0004525 |
| 4395307 | (SEQ ID NO: 198) | |
| rno-miR-463- | UGAUAGACGCCAAUUUGGGUAG | MIMAT0005317 |
| 4395751 | (SEQ ID NO: 199) | |
| rno-miR-664- | UAUUCAUUUACUCCCCAGCCUA | MIMAT0003382 |
| 4381103 | (SEQ ID NO: 200) | |
| rno-miR-743a- | GAAAGACGCCAAACUGGGUAGA | MIMAT0005334 |
| 4395757 | (SEQ ID NO: 201) | |
| snoRNA135- | CTAAAATAGCTGGAATTACCGG | Mature |
| 4380912 | CAGATTGGTAGTGGTGAGCCTA | miRNA |
| | TGGTTTTCTGAAG | Control |
| | (SEQ ID NO: 202) | |
| U87- | ACAATGATGACTTATGTTTTTG | Mature |
| 4386735 | CCGTTTACCCAGCTGAGGGTTT | miRNA |
| | CTTTGAAGAGAGAATC | Control |
| | TTAAGACTGAGC | |
| | (SEQ ID NO: 203) | |

Table 8 depicts results from analysis of miRNAs from rat PC RNA preparations.

TABLE 8
Exemplary miRNA sequences in serum starved rat PCs

| | | Alternative |
|---|---|---|
| miRNA | Exemplary Sequence(s)(5′ to 3′) | Description |
| mmu-miR-101a- | UACAGUACUGUGAUAACUGAAG (SEQ ID | MIMAT0000133 |
| 4395364 | NO: 204) | |
| mmu-miR-10a- | UACCCUGUAGAUCCGAAUUUGUG (SEQ ID | MIMAT0000648 |
| 4373153 | NO: 205) | |
| mmu-miR-10b- | UACCCUGUAGAACCGAAUUUGUG (SEQ ID | MIMAT0000208 |
| 4395329 | NO: 206) | |
| mmu-miR-125a-3p- | ACAGGUGAGGUUCUUGGGAGCC (SEQ ID | MIMAT0004528 |
| 4395310 | NO: 207) | |
| mmu-miR-125a-5p- | UCCCUGAGACCCUUUAACCUGUGA | MIMAT0000135 |
| 4395309 | (SEQ ID NO: 208) | |
| mmu-miR-125b-3p- | ACGGGUUAGGCUCUUGGGAGCU (SEQ ID | MIMAT0004669 |
| 4395489 | NO: 209) | |
| mmu-miR-128a- | UCACAGUCAACCGGUCUCUUU | MIMAT0000424 |
| 4395327 | (SEQ ID NO: 210) | |
| mmu-miR-129-3p- | AAGCCCUUACCCCAAAAAGCAU (SEQ ID | MIMAT0000544 |
| 4373297 | NO: 211) | |
| mmu-miR-138- | AGCUGGUGUUGUGAAUCAGGCCG (SEQ ID | MIMAT0000150 |
| 4395395 | NO: 212) | |
| mmu-miR-142-3p- | UGUAGUGUUUCCUACUUUAUGGA (SEQ ID | MIMAT0000155 |
| 4373136 | NO: 213) | |
| mmu-miR-142-5p- | CAUAAAGUAGAAAGCACUACU (SEQ ID | MIMAT0000154 |
| 4395359 | NO: 214) | |
| mmu-miR-143- | UGAGAUGAAGCACUGUAGCUC (SEQ ID | MIMAT0000247 |
| 4395360 | NO: 215) | |
| mmu-miR-146a- | UGAGAACUGAAUUCCAUGGGUU (SEQ ID | MIMAT0000158 |
| 4373132 | NO: 216) | |
| mmu-miR-147- | GUGUGCGGAAAUGCUUCUGCUA (SEQ ID | MIMAT0004857 |
| 4395373 | NO: 217) | |
| mmu-miR-148a- | UCAGUGCACUACAGAACUUUGU (SEQ ID | MIMAT0000516 |
| 4373130 | NO: 218) | |
| mmu-miR-148b- | UCAGUGCAUCACAGAACUUUGU (SEQ ID | MIMAT0000580 |
| 4373129 | NO: 219) | |
| mmu-miR-151-3p- | CUAGACUGAGGCUCCUUGAGG (SEQ ID | MIMAT0000161 |
| 4373304 | NO: 220) | |
| mmu-miR-182- | UUUGGCAAUGGUAGAACUCACACCG (SEQ | MIMAT0000211 |
| 4395729 | ID NO: 221) | |
| mmu-miR-187- | UCGUGUCUUGUGUUGCAGCCGG (SEQ ID | MIMAT0000216 |
| 4373307 | NO: 222) | |
| mmu-miR-188-5p- | CAUCCCUUGCAUGGUGGAGGG (SEQ ID | MIMAT0000217 |
| 4395431 | NO: 223) | |
| mmu-miR-18a- | UAAGGUGCAUCUAGUGCAGAUAG (SEQ ID | MIMAT0000528 |
| 4395533 | NO: 224) | |
| mmu-miR-190- | UGAUAUGUUUGAUAUAUUAGGU (SEQ ID | MIMAT0000220 |
| 4373110 | NO: 225) | |
| mmu-miR-196b- | UAGGUAGUUUCCUGUUGUUGGG (SEQ ID | MIMAT0001081 |
| 4395326 | NO: 226) | |
| mmu-miR-197- | UUCACCACCUUCUCCACCCAGC | MIMAT0000227 |
| 4373102 | (SEQ ID NO: 227) | |
| mmu-miR-199a-3p- | ACAGUAGUCUGCACAUUGGUUA (SEQ ID | MIMAT0000230 |
| 4395415 | NO: 228) | |
| mmu-miR-200c- | UAAUACUGCCGGGUAAUGAUGGA (SEQ ID | MIMAT0000657 |
| 4395411 | NO: 229) | |
| mmu-miR-204- | UUCCCUUUGUCAUCCUAUGCCU (SEQ ID | MIMAT0000237 |
| 4373094 | NO: 230) | |
| mmu-miR-210- | CUGUGCGUGUGACAGCGGCUGA (SEQ ID | MIMAT0000658 |
| 4373089 | NO: 231) | |
| mmu-miR-21- | UAGGUUAUCAGACUGAUGUUGA (SEQ ID | MIMAT0000530 |
| 4373090 | NO: 232) | |
| mmu-miR-222- | AGCUACAUCUGGCUACUGGGU (SEQ ID | MIMAT0000670 |
| 4395387 | NO: 233) | |
| mmu-miR-23a- | AUCACAUUGCCAGGGAUUUCC (SEQ ID | MIMAT0000532 |
| 4373074 | NO: 234) | |
| mmu-miR-23b- | AUCACAUUGCCAGGGAUUACC (SEQ ID | MIMAT0000125 |
| 4373073 | NO: 235) | |
| mmu-miR-26a- | UUCAAGUAAUCCAGCAUAGGCU (SEQ ID | MIMAT0000533 |
| 4395166 | NO: 236) | |
| mmu-miR-29b- | UAGCACCAUUUGAAAUCAGUGUU (SEQ ID | MIMAT0000127 |
| 4373288 | NO: 237) | |
| mmu-miR-29c- | UAGCACCAUUUGAAAUCGGUUA (SEQ ID | MIMAT0000536 |
| 4395171 | NO: 238) | |
| mmu-miR-320- | AAAAGCUGGGUUGAGAGGGCGA (SEQ ID | MIMAT0000666 |
| 4395388 | NO: 239) | |
| mmu-miR-322- | CAGCAGCAAUUCAUGUUUUGGA (SEQ ID | MIMAT0000548 |
| 4378107 | NO: 240) | |
| mmu-miR-324-5p- | CGCAUCCCCUAGGGCAUUGGUGU (SEQ ID | MIMAT0000555 |
| 4373052 | NO: 241) | |
| mmu-miR-331-5p- | CUAGGUAUGGUCCCAGGGAUCC (SEQ ID | MIMAT0004643 |
| 4395344 | NO: 242) | |
| mmu-miR-335-3p- | UUUUUCAUUAUUGCUCCUGACC (SEQ ID | MIMAT0004704 |
| 4395296 | NO: 243) | |
| mmu-miR-339-5p- | UCCCUGUCCUCCAGGAGCUCACG (SEQ ID | MIMAT0000584 |
| 4395368 | NO: 244) | |
| mmu-miR-345-5p- | GCUGACCCCUAGUCCAGUGCUU (SEQ ID | MIMAT0000595 |
| 4395658 | NO: 245) | |
| mmu-miR-350- | UUCACAAAGCCCAUACACUUUC (SEQ ID | MIMAT0000605 |
| 4395660 | NO: 246) | |
| mmu-miR-351- | UCCCUGAGGAGCCCUUUGAGCCUG (SEQ ID | MIMAT0000609 |
| 4373345 | NO: 247) | |
| mmu-miR-361- | UUAUCAGAAUCUCCAGGGGUAC (SEQ ID | MIMAT0000704 |
| 4373035 | NO: 248) | |
| mmu-miR-362-3p- | AACAGACCUGUUGAAGGAUUCA (SEQ ID | MIMAT0004684 |
| 4395746 | NO: 249) | |
| mmu-miR-384-5p- | UGUAAACAAUUCCUAGGCAAUGU (SEQ ID | MIMAT0004745 |
| 4395732 | NO: 250) | |
| mmu-miR-429- | UAAUACUGUCUGGUAAUGCCGU (SEQ ID | MIMAT0001537 |
| 4373355 | NO: 251) | |
| mmu-miR-450a-5p- | UUUUGCGAUGUGUUCCUAAUAU (SEQ ID | MIMAT0001546 |
| 4395414 | NO: 252) | |
| mmu-miR-494- | UGAAACAUACACGGGAAACCUC (SEQ ID | MIMAT0003182 |
| 4395476 | NO: 253) | |
| mmu-miR-500- | AAUGCACCUGGGCAAGGGUUCA (SEQ ID | MIMAT0003507 |
| 4395736 | NO: 254) | |
| mmu-miR-503- | UAGCAGCGGGAACAGUACUGCAG (SEQ ID | MIMAT0003188 |
| 4395586 | NO: 255) | |
| mmu-miR-542-3p- | UGUGACAGAUUGAUAACUGAAA (SEQ ID | MIMAT0003172 |
| 4378101 | NO: 256) | |
| mmu-miR-582-3p- | CCUGUUGAACAACUGAACCCAA (SEQ ID | MIMAT0005292 |
| 4395697 | NO: 257) | |
| mmu-miR-582-5p- | UACAGUUGUUCAACCAGUUACU (SEQ ID | MIMAT0005291 |
| 4395696 | NO: 258) | |
| mmu-miR-598- | UACGUCAUCGUCGUCAUCGUUA (SEQ ID | MIMAT0004942 |
| 4395606 | NO: 259) | |
| mmu-miR-652- | AAUGGCGCCACUAGGGUUGUG (SEQ ID | MIMAT0003711 |
| 4395463 | NO: 260) | |
| mmu-miR-667- | UGACACCUGCCACCGAGCCCAAG (SEQ ID | MIMAT0003734 |
| 4386769 | NO: 261) | |
| mmu-miR-685- | UCAAUGGCUGAGGUGAGGCAC | MIMAT0003463 |
| 4386748 | (SEQ ID NO: 262) | |
| mmu-miR-743b-5p- | UGUUCAGACUGGUGUCCAUCA (SEQ ID | MIMAT0004839 |
| 4395600 | NO: 263) | |
| mmu-miR-744- | UGCGGGGCUAGGGCUAACAGCA (SEQ ID | MIMAT0004187 |
| 4395435 | NO: 264) | |
| mmu-miR-883a-3p- | UAACUGCAACAGCUCUCAGUAU (SEQ ID | MIMAT0004849 |
| 4395591 | NO: 265) | |
| mmu-miR-883b-3p- | UAACUGCAACAUCUCUCAGUAU (SEQ ID | MIMAT0004851 |
| 4395695 | NO: 266) | |
| mmu-miR-98- | UGAGGUAGUAAGUUGUAUUGUU (SEQ ID | MIMAT0000545 |
| 4373009 | NO: 267) | |
| rno-miR-190b- | UGAUAUGUUUGAUAUUAGGUU (SEQ ID | MIMAT0005302 |
| 4395749 | NO: 268) | |
| rno-miR-207- | GCUUCUCCUGGCUCUCCUCCCUU (SEQ ID | MIMAT0003115 |
| 4381096 | NO: 269) | |
| rno-miR-333- | GUGGUGUGCUAGUUACUUUU | |
| 4381109 | (SEQ ID NO: 270) | |
| rno-miR-339-3p- | UGAGCGCCUCGACGACAGAGCCA (SEQ ID | MIMAT0004648 |
| 4395760 | NO: 271) | |
| rno-miR-345-3p- | CCCUGAACUAGGGGUCUGGAGA (SEQ ID | MIMAT0004655 |
| 4395762 | NO: 272) | |
| rno-miR-351- | UCCCUGAGGAGCCCUUUGAGCCUGA (SEQ | MIMAT0000608 |
| 4395764 | ID NO: 273) | |
| rno-miR-466e- | UGUGAUGUGUGCAUGUACAUG (SEQ ID | MIMAT0005279 |
| 4395768 | NO: 274) | |
| rno-miR-743b- | GAAAGACACCAUACUGAAUAGA (SEQ ID | MIMAT0005280 |
| 4395769 | NO: 275) | |
| snoRNA202-4380914 | GCTGTACTGACTTGATGAAAGTACTTTTGA | |
| | ACCCTTTTCCATCTGATG | |
| | (SEQ ID NO: 276) | |
| mmu-let-7f*- | CUAUACAAUCUAUUGCCUUCCC (SEQ ID | MIMAT0004623 |
| 4395528 | NO: 277) | |
| mmu-let-7g*- | ACUGUACAGGCCACUGCCUUGC (SEQ ID | MIMAT0004519 |
| 4395622 | NO: 278) | |
| mmu-let-7i*- | CUGCGCAAGCUACUGCCUUGGU (SEQ ID | MIMAT0004520 |
| 4395283 | NO: 279) | |
| mmu-miR-106b*- | CCGCACUGUGGGUACUUGCUGC (SEQ ID | MIMAT0004582 |
| 4395491 | NO: 280) | |
| mmu-miR-10a*- | CAAAUUCGUAUCUAGGGGAAUA (SEQ ID | MIMAT0004659 |
| 4395399 | NO: 281) | |
| mmu-miR-10b*- | CAGAUUCGAUUCUAGGGGAAUA (SEQ ID | MIMAT0004538 |
| 4395702 | NO: 282) | |
| mmu-miR-130b*- | ACUCUUUCCCUGUUGCACUACU (SEQ ID | MIMAT0004583 |
| 4395590 | NO: 283) | |
| mmu-miR-135a*- | UAUAGGGAUUGGAGCCGUGGCG (SEQ ID | MIMAT0004531 |
| 4395343 | NO: 284) | |
| mmu-miR-149- | UCUGGCUCCGUGUCUUCACUCCC (SEQ ID | MIMAT0000159 |
| 4395366 | NO: 285) | |
| mmu-miR-15b*- | CGAAUCAUUAUUUGCUGCUCUA (SEQ ID | MIMAT0004521 |
| 4395284 | NO: 286) | |
| mmu-miR-16*- | CCAGUAUUGACUGUGCUGCUGA (SEQ ID | MIMAT0004625 |
| 4395619 | NO: 287) | |
| mmu-miR-17*- | ACUGCAGUGAGGGCACUUGUAG (SEQ ID | MIMAT0000650 |
| 4395673 | NO: 288) | |
| mmu-miR-18a*- | ACUGCCCUAAGUGCUCCUUCUG (SEQ ID | MIMAT0004626 |
| 4395620 | NO: 289) | |
| mmu-miR-191*- | GCUGCACUUGGAUUUCGUUCCC (SEQ ID | MIMAT0004542 |
| 4395706 | NO: 290) | |
| mmu-miR-199b*- | CCCAGUGUUUAGACUACCUGUUC (SEQ ID | MIMAT0000672 |
| 4373309 | NO: 291) | |
| mmu-miR-206- | UGGAAUGUAAGGAAGUGUGUGG (SEQ ID | MIMAT0000239 |
| 4373092 | NO: 292) | |
| mmu-miR-214*- | UGCCUGUCUACACUUGCUGUGC (SEQ ID | MIMAT0004664 |
| 4395404 | NO: 293) | |
| mmu-miR-218-1*- | AAACAUGGUUCCGUCAAGCACC (SEQ ID | MIMAT0004665 |
| 4395682 | NO: 294) | |
| mmu-miR-24-1*- | GUGCCUACUGAGCUGAUAUCAGU (SEQ ID | MIMAT0000218 |
| 4378067 | NO: 295) | |
| mmu-miR-26b*- | CCUGUUCUCCAUUACUUGGCUC (SEQ ID | MIMAT0004630 |
| 4395555 | NO: 296) | |
| mmu-miR-291a-5p- | CAUCAAAGUGGAGGCCCUCUCU (SEQ ID | MIMAT0000367 |
| 4373322 | NO: 297) | |
| mmu-miR-297a*- | UAUACAUACACACAUACCCAUA (SEQ ID | MIMAT0004864 |
| 4395584 | NO: 298) | |
| mmu-miR-29a*- | ACUGAUUUCUUUUGGUGUUCAG (SEQ ID | MIMAT0004631 |
| 4395558 | NO: 299) | |
| mmu-miR-29b*- | GCUGGUUUCAUAUGGUGGUUUA (SEQ ID | MIMAT0004523 |
| 4395627 | NO: 300) | |
| mmu-miR-29c*- | UGACCGAUUUCUCCUGGUGUUC (SEQ ID | MIMAT0004632 |
| 4381131 | NO: 301) | |
| mmu-miR-30a*- | CUUUCAGUCGGAUGUUUGCAGC (SEQ ID | MIMAT0000129 |
| 4373062 | NO: 302) | |
| mmu-miR-30b*- | CUGGGAUGUGGAUGUUUACGUC (SEQ ID | MIMAT0004524 |
| 4395628 | NO: 303) | |
| mmu-miR-30c-1*- | CUGGGAGAGGGUUGUUUACUCC (SEQ ID | MIMAT0004616 |
| 4395219 | NO: 304) | |
| mmu-miR-30e*- | CUUUCAGUCGGAUGUUUACAGC (SEQ ID | MIMAT0000249 |
| 4373057 | NO: 305) | |
| mmu-miR-322*- | AAACAUGAAGCGCUGCAACAC (SEQ ID | MIMAT0000549 |
| 4395636 | NO: 306) | |
| mmu-miR-326- | CCUCUGGGCCCUUCCUCCAGU (SEQ ID | MIMAT0000559 |
| 4373335 | NO: 307) | |
| mmu-miR-330*- | GCAAAGCACAGGGCCUGCAGAGA (SEQ ID | MIMAT0000569 |
| 4373337 | NO: 308) | |
| mmu-miR-374- | AUAUAAUACAACCUGCUAAGUG (SEQ ID | MIMAT0003727 |
| 4381045 | NO: 309) | |
| mmu-miR-378*- | CUCCUGACUCCAGGUCCUGUGU (SEQ ID | MIMAT0000742 |
| 4373024 | NO: 389) | |
| mmu-miR-378- | ACUGGACUUGGAGUCAGAAGG (SEQ ID | MIMAT0003151 |
| 4395354 | NO: 310) | |
| mmu-miR-425*- | AUCGGGAAUGUCGUGUCCGCC (SEQ ID | MIMAT0001342 |
| 4373202 | NO: 311) | |
| mmu-miR-466d-3p- | UAUACAUACACGCACACAUAG (SEQ ID | MIMAT0004931 |
| 4395665 | NO: 312) | |
| mmu-miR-467a*- | AUAUACAUACACACACCUACAC (SEQ ID | MIMAT0002108 |
| 4386757 | NO: 313) | |
| mmu-miR-467b*- | AUAUACAUACACACACCAACAC (SEQ ID | MIMAT0003478 |
| 4381092 | NO: 314) | |
| mmu-miR-503*- | GAGUAUUGUUUCCACUGCCUGG (SEQ ID | MIMAT0004790 |
| 4395666 | NO: 315) | |
| mmu-miR-673-5p- | CUCACAGCUCUGGUCCUUGGAG (SEQ ID | MIMAT0003739 |
| 4386772 | NO: 316) | |
| mmu-miR-674*- | CACAGGUCCCAUCUCAGAACAA (SEQ ID | MIMAT0003741 |
| 4386773 | NO: 317) | |
| mmu-miR-678- | GUCUCGGUGCAAGGACUGGAGG (SEQ ID | MIMAT0003452 |
| 4381076 | NO: 318) | |
| mmu-miR-692- | AUCUCUUUGAGCGCCUCACUC (SEQ ID | MIMAT0003471 |
| 4381088 | NO: 319) | |
| mmu-miR-699- | AGGCAGUGCGACCUGGCUCG | MIMAT0003489 |
| 4381056 | (SEQ ID NO: 320) | |
| mmu-miR-720- | AUCUCGCUGGGGCCUCCA | MIMAT0003484 |
| 4381052 | (SEQ ID NO: 321) | |
| mmu-miR-721- | CAGUGCAUUAAAAGGGGGAA (SEQ ID | MIMAT0003515 |
| 4381073 | NO: 322) | |
| mmu-miR-744*- | CUGUUGCCACUAACCUCAACCU (SEQ ID | MIMAT0004820 |
| 4395436 | NO: 323) | |
| mmu-miR-760- | CGGCUCUGGGUCUGUGGGGA (SEQ ID | MIMAT0003898 |
| 4395439 | NO: 324) | |
| mmu-miR-801- | GAUUGCUGUGCGUGCGGAAUCGAC | |
| 4395562 | (SEQ ID NO: 325) | |
| mmu-miR-875-5p- | UAUACCUCAGUUUUAUCAGGUG (SEQ ID | MIMAT0004937 |
| 4395314 | NO: 326) | |
| mmu-miR-877- | GUAGAGGAGAUGGCGCAGGG (SEQ ID | MIMAT0004861 |
| 4395402 | NO: 327) | |
| mmu-miR-9*- | AUAAAGCUAGAUAACCGAAAGU (SEQ ID | MIMAT0000143 |
| 4395342 | NO: 328) | |
| mmu-miR-99b*- | CAAGCUCGUGUCUGUGGGUCCG (SEQ ID | MIMAT0004525 |
| 4395307 | NO: 329) | |
| rno-miR-28*- | CACUAGAUUGUGAGCUCCUGGA (SEQ ID | MIMAT0004716 |
| 4395557 | NO: 330) | |
| rno-miR-463- | UGAUAGACGCCAAUUUGGGUAG (SEQ ID | MIMAT0005317 |
| 4395751 | NO: 331) | |
| rno-miR-99a*- | CAA.GCUCGUUUCUAUGGGUCUG (SEQ ID | MIMAT0004724 |
| 4395774 | NO: 332) | |
| snoRNA1.35-4380912 | CTAAAATAGCTGGAATTACCGGCAGATTGG | Mature miRNA |
| | TAGTGGTGAGCCTATGGTTTTCTGAAG | Control |
| | (SEQ ID NO: 333) | |

Table 9 lists miRNAs in common between rat PCs grown under serum starvation conditions (identified in Table 8) and MVs from rat PCs grown under serum starvation conditions (identified in Table 7).

TABLE 9
miRNA sequences in both serum starved rat PDPCs and MVs from serum
starved rat PDPCs

| miRNA in MVs | | Alternative |
|---|---|---|
| from Rat PCs | Exemplary Sequence(s) (5′ to 3′) | Description |
| mmu-miR-146a- | UGAGAACUGAAUUCCAUGGGUU (SEQ | MIMAT0000158 |
| 4373132 | ID NO: 334) | |
| mmu-miR-188-5p- | CAUCCCUUGCAUGGUGGAGGG (SEQ | MIMAT0000217 |
| 4395431 | ID NO: 335) | |
| mmu-miR-196b- | UAGGUAGUUUCCUGUUGUUGGG (SEQ | MIMAT0001081 |
| 4395326 | ID NO: 336) | |
| mmu-miR-204- | UUCCCUUUGUCAUCCUAUGCCU (SEQ | MIMAT0000237 |
| 4373094 | ID NO: 337) | |
| mmu-miR-210- | CUGUGCGUGUGACAGCGGCUGA (SEQ | MIMAT0000658 |
| 4373089 | ID NO: 338) | |
| mmu-miR-23b- | AUCACAUUGCCAGGGAUUACC (SEQ | MIMAT0000125 |
| 4373073 | ID NO: 339) | |
| mmu-miR-29c- | UAGCACCAUUUGAAAUCGGUUA (SEQ | MIMAT0000536 |
| 4395171 | ID NO: 340) | |
| mmu-miR-320- | AAAAGCUGGGUUGAGAGGGCGA (SEQ | MIMAT0000666 |
| 4395388 | ID NO: 341) | |
| mmu-miR-335-3p- | UUUUUCAUUAUUGCUCCUGACC (SEQ | MIMAT0004704 |
| 4395296 | ID NO: 342) | |
| mmu-miR-652- | AAUGGCGCCACUAGGGUUGUG (SEQ | MIMAT0003711 |
| 4395463 | ID NO: 343) | |
| mmu-miR-135a*- | UAUAGGGAUUGGAGCCGUGGCG (SEQ | MIMAT0004531 |
| 4395343 | ID NO: 344) | |
| mmu-miR-206- | UGGAAUGUAAGGAAGUGUGUGG (SEQ | MIMAT0000239 |
| 4373092 | ID NO: 345) | |
| mmu-miR-26b*- | CCUGUUUCCAUUACUUGGCUC (SEQ | MIMAT0004630 |
| 4395555 | ID NO: 346) | |
| mmu-miR-29b*- | GCUGGUUUCAUAUGGUGGUUUA | MIMAT0004523 |
| 4395627 | (SEQ ID NO: 347) | |
| mmu-miR-378- | ACUGGACUUGGAGUCAGAAGG (SEQ | MIMAT0003151 |
| 4395354 | ID NO: 348) | |
| mmu-miR-466d- | UAUACAUACACGCACACAUAG (SEQ | MIMAT0004931 |
| 32-4395665 | ID NO: 349) | |
| mmu-miR-467b*- | AUAUACAUACACACACCAACAC (SEQ | MIMAT0003478 |
| 4381092 | ID NO: 350) | |
| mmu-miR-673-5p- | CUCACAGCUCUGGUCCUUGGAG (SEQ | MIMAT0003739 |
| 4386772 | ID NO: 351) | |
| mmu-miR-674*- | CACAGCUCCCAUCUCAGAACAA (SEQ | MIMAT0003741 |
| 4386773 | ID NO: 352) | |
| mmu-miR-720- | AUCUCGCUGGGGCCUCCA (SEQ ID | MIMAT0003484 |
| 4381052 | NO: 353) | |
| mmu-miR-721- | CAGUGCAAUUAAAAGGGGGAA (SEQ | MIMAT0003515 |
| 4381073 | ID NO: 354) | |
| mmu-miR-760- | CGGCUCUGGGUCUGUGGGGA (SEQ ID | MIMAT0003898 |
| 4395439 | NO: 355) | |
| mmu-miR-801- | GAUUGCUGUGCGUGCGGAAUCGAC | |
| 4395562 | SEQ ID NO: 356) | |
| mmu-miR-877- | GUAGAGGAGAUGGCGCAGGG (SEQ ID | MIMAT0004861 |
| 4395402 | NO: 357) | |
| mmu-miR-99b*- | CAAGGUCGUGUCUGUGGGUCCG (SEQ | MIMAT0004525 |
| 4395307 | ID NO: 358) | |
| snoRNA135- | CTAAAATAGCTGGAATTACCGGCAGAT | Mature miRNA Control |
| 4380912 | TGG | |
| | TAGTGGTGAGCCTATGGTTTTCTGAAG | |
| | (SEQ ID NO: 359) | |

Table 10 lists miRNAs found in rat PC MVs, including exosomes.

TABLE 10
miRNAs found in rat PC04 MV (including exosomes)

| miRNA in MVs | | Alternative |
|---|---|---|
| from Rat PDPCs | Exemplary Sequence(s) (5′ to 3′) | Description |
| mmu-miR-106a- | CAAAGUGCUAACAGUGCAGGUA | MIMAT0000385 |
| 4395589 | G (SEQ ID NO: 360) | |
| mmu-miR-106b- | UAAAGUGCUGACAGUGCAGAU | MIMAT0000386 |
| 4373155 | (SEQ ID NO: 361) | |
| mmu-miR-10a- | UACCCUGUAGAUCCGAAUUUGU | MIMAT0000648 |
| 4373153 | G (SEQ ID NO: 362) | |
| mmu-miR-1263p- | UCGUACCGUGAGUAAUAAUGCG | MIMAT0000138 |
| 4395339 | (SEQ ID NO: 363) | |
| mmu-miR-130a- | CAGUGCAAUGUUAAAAGGGCAU | MIMAT0000141 |
| 4373145 | (SEQ ID NO: 364) | |
| mmu-miR-140- | CAGUGGUUUUACCCUAUGGUAG | MIMAT0000151 |
| 4373374 | (SEQ ID NO: 365) | |
| mmu-miR-145- | GUCCAGUUUUCCGAGGAAUCCCU | MIMAT0000157 |
| 4395389 | (SEQ ID NO: 366) | |
| mmu-miR-146a- | UGAGAACUGAAUUCCAUGGGUU | MIMAT0000158 |
| 4373132 | (SEQ ID NO: 367) | |
| mmu-miR-146b- | UGAGAACUGAAUUCCAUAGGCU | MIMAT0003475 |
| 4373178 | (SEQ ID NO: 368) | |
| mmu-miR-155- | UUAAUGCUAAUUGUGAUAGGGG | MLMAT0000165 |
| 4395701 | U (SEQ ID NO: 369) | |
| mmu-miR-15b- | UAGCAGGACAUCAUGGUUUACA | MIMAT0000124 |
| 4373122 | (SEQ ID NO: 370) | |
| mmu-miR-16- | UAGCAGCACGUAAAUAUUGGCG | MIMAT0000527 |
| 4373121 | (SEQ ID NO: 371) | |
| mmu-miR-181a- | AACAUUCAACGCUGUCGGUGAG | MIMAT0000210 |
| 4373117 | U (SEQ ID NO: 372) | |
| mmu-miR-188- | CAUCCCUUGCAUGGUGGAGGG | MIMAT0000217 |
| 5p-4395431 | (SEQ ID NO: 373) | |
| mmu-miR-196b- | UAGGUAGUUUCCUGUUGUUGGG | MIMAT0001081 |
| 4395326 | (SEQ ID NO: 374) | |
| mmu-miR-19a- | UGUGCAAAUCUAUGCAAAACUG | MIMAT0000651 |
| 4373099 | A (SEQ ID NO: 375) | |
| mmu-miR-204- | UUCCCUUUGUCAUCCUAUGCCU | MIMAT0000237 |
| 4373094 | (SEQ ID NO: 376) | |
| mmu-miR-20a- | UAAAGUGCUUAUAGUGCAGGUA | MIMAT000529 |
| 4373286 | G (SEQ ID NO: 377) | |
| mmu-miR-210- | CUGUGCGUGUGACAGCGGCUGA | MIMAT0000658 |
| 4373089 | (SEQ ID NO: 378) | |
| mmu-miR-21- | UAGCUUAUCAGACUGAUGUUGA | MIMAT0000530 |
| 4373090 | (SEQ ID NO: 379) | |
| mmu-miR-218- | UUGUGCUUGAUGUAACCAUGU | MIMAT0000663 |
| 4373081 | (SEQ ID NO: 380) | |
| mmu-miR-23b- | AUCACAUUGCCAGGGAUUACC | MIMAT0000125 |
| 4373073 | (SEQ ID NO: 381) | |
| mmu-miR-24- | UGGCUCAGUUCAGCAGGAACAG | MIMAT0000219 |
| 4373072 | (SEQ ID NO: 382) | |
| mmu-miR-25- | CAUUGCACUUGUCUCGGUCUGA | MIMAT0000652 |
| 4373071 | (SEQ ID NO: 383) | |
| mmu-miR-27b- | UUCACAGUGGCUAAGUUCUGC | MIMAT0000126 |
| 4373068 | (SEQ ID NO: 384) | |
| mmu-miR-29c- | UAGCACCAUUUGAAAUCGGUUA | MIMAT0000536 |
| 4395171 | (SEQ ID NO: 385) | |
| mmu-miR-30c- | UGUAAACAUCCUACACUCUCAGC | MIMAT0000514 |
| 4373060 | (SEQ ID NO: 386) | |
| mmu-miR-30d- | UGUAAACAUCCCCGACUGGAAG | MIMAT0000515 |
| 4373059 | (SEQ ID NO: 387) | |
| mmu-miR-30e- | UGUAAACAUCCUUGACUGGAAG | MIMAT0000248 |
| 4395334 | (SEQ ID NO: 388) | |
| mmu-miR-320- | AAAAGCUGGGUUGAGAGGGCGA | MIMAT0000666 |
| 4395388 | (SEQ ID NO: 390) | |
| mmu-miR-324-3p- | CCACUGCCCCAGGUGGUGCU | MIMAT0000556 |
| 4395639 | (SEQ ID NO: 391) | |
| mmu-miR-328- | CUGGCCCUCUCUGCCCUUCCGU | MIMAT0000565 |
| 4373049 | (SEQ ID NO: 392) | |
| mmu-miR-331-3p- | GCCCCUGGGCCUAUCCUAGAA | MIMAT0000571 |
| 4373046 | (SEQ ID NO: 393) | |
| mmu-miR-335-3p- | UUUUUCAUUAUUGCUCCUGACC | MIMAT0004704 |
| 4395296 | (SEQ ID NO: 394) | |
| mmu-miR-410- | AAUAUAACACAGAUGGCCUGU | MIMAT0001091 |
| 4378093 | (SEQ ID NO: 395) | |
| mmu-miR-434-3p- | UUUGAACCAUCACUCGACUCCU | MIMAT0001422 |
| 4395734 | (SEQ ID NO: 396) | |
| mmu-miR-574-3p- | CACGCUCAUGCACACACCCACA | MIMAT0004894 |
| 4395460 | (SEQ ID NO: 397) | |
| mmu-miR-652- | AAUGGCGCCACUAGGGUUGUG | MIMAT0003711 |
| 4395463 | (SEQ ID NO: 398) | |
| mmu-miR-93- | CAAAGUGGUGUUCGUGCAGGUA | MLMAT0000540 |
| 4373302 | G (SEQ ID NO: 399) | |
| mmu-miR-99b- | CACCCGUAGAACCGACCUUGCG | MIMAT0000132 |
| 4373007 | (SEQ ID NO: 400) | |
| mmu-miR-196c- | UAGGUAGUUUCGUGUUGUUGGG | MIMAT0005303 |
| 4395750 | (SEQ ID NO: 401) | |
| mmu-let-7a*- | CUAUACAAUCUACUGUCUUUCC | MIMAT0004620 |
| 4395608 | (SEQ ID NO: 402) | |
| mmu-miR-125b*- | ACAAGUCAGGUUCUUGGGACCU | MIMAT0004529 |
| 4395638 | (SEQ ID NO: 403) | |
| mmu-miR-135a*- | UAUAGGGAUUGGAGCCGUGGCG | MIMAT0004531 |
| 4395343 | (SEQ ID NO: 404) | |
| mmu-miR-136*- | AUCAUCGUCUCAAAUGAGUCUU | MIMAT0004532 |
| 4395642 | (SEQ ID NO: 405) | |
| mmu-miR-138*- | CGGCUACUUCACAACACCAGGG | MIMAT0004668 |
| 4395684 | (SEQ ID NO: 406) | |
| mmu-miR-141*- | CAUCUUCCAGUGCAGUGUUGGA | MIMAT0004533 |
| 4395643 | (SEQ ID NO: 407) | |
| mmu-miR-186*- | GCCCUAAGGUGAAUUUUUUGGG | MIMAT0004540 |
| 4395704 | (SEQ ID NO: 408) | |
| mmu-miR-190b- | UGAUAUGUUUGAUAUUGGGUU | MIMAT0004852 |
| 4395374 | (SEQ ID NO: 409) | |
| mmu-miR-206- | UGGAAUGUAAGGAAGUGUGUGG | MIMAT0000239 |
| 4373092 | (SEQ ID NO: 410) | |
| mmu-miR-26b*- | CCUGUUCUCCAUUACUUGGCUC | MIMAT0004630 |
| 4395555 | (SEQ ID NO: 411) | |
| mmu-miR-29b*- | GCUGGUUUCAUAUGGUGGUUUA | MIMAT0004523 |
| 4395627 | (SEQ ID NO: 412) | |
| mmu-miR-340- | AAUCAGUAACCACACAGCCAGG | MIMAT0004580 |
| 4395714 | (SEQ ID NO: 413) | |
| mmu-miR-378- | ACUGGACUUGGAGUCAGAAGG | MIMAT0003151 |
| 4395354 | (SEQ ID NO: 414) | |
| mmu-miR-466d-3p- | UAUACAUACACGCACACAUAG | MIMAT0004931 |
| 4395665 | (SEQ ID NO: 415) | |
| mmu-miR-467b*- | AUAUACAUACACACACCAACAC | MIMAT0003478 |
| 4381092 | (SEQ ID NO: 416) | |
| mmu-miR-673-5p- | CUCACAGCUCUGGUCCUUGGAG | MIMAT0003739 |
| 4386772 | (SEQ ID NO: 417) | |
| mmu-miR-674*- | CACAGCUCCCAUCUCAGAACAA | MIMAT0003741 |
| 4386773 | (SEQ ID NO: 418) | |
| mmu-miR-690- | AAAGGCUAGGCUCACAACCAAA | MIMAT0003469 |
| 4381086 | (SEQ ID NO: 419) | |
| mmu-miR-696- | GCGUGUGGUUGCUGUGGG | MIMAT0003483 |
| 4381051 | (SEQ ID NO: 420) | |
| mmu-miR-697- | AACAUCCUGGUCCUGUGGAGA | MIMAT0003487 |
| 4381054 | (SEQ ID NO: 421) | |
| mmu-miR-715- | CUCCGUGCACACCCCCGCGUG | MIMAT0003506 |
| 4381067 | (SEQ ID NO: 422) | |
| mmu-miR-720- | AUCUCGCUGGGGCCUCCA (SEQ | MIMAT0003484 |
| 4381052 | ID NO: 423) | |
| mmu-miR-721- | CAGUGCAAUUAAAAGGGGGAA | MIMAT0003515 |
| 4381073 | (SEQ ID NO: 424) | |
| mmu-miR-760- | CGGCUCUGGGUCUGUGGGGA | MIMAT0000898 |
| 4395439 | (SEQ ID NO : 425) | |
| mmu-miR-801- | GAUUGCUGUGCGUGCGGAAUCG | |
| 4395562 | AC (SEQ ID NO: 426) | |
| mmu-miR-805- | GAAUUGAUCAGGACAUAGGG | MIMAT0004211 |
| 4395577 | (SEQ ID NO: 427) | |
| mmu-miR-872*- | UGAACUAUUGCAGUAGCCUCCU | MIMAT0004935 |
| 4395672 | (SEQ ID NO: 428) | |
| mmu-miR-877*- | UGUCCUCUUCUCCCUCCUCCCA | MIMAT0004862 |
| 4395678 | (SEQ ID NO: 429) | |
| mmu-miR-877- | GUAGAGGAGAUGGCGCAGGG | MIMAT0004861 |
| 4395402 | (SEQ ID NO: 430) | |
| mmu-miR-878-3p- | GCAUGACACCACACUGGGUAGA | MIMAT0004933 |
| 4395671 | (SEQ ID NO: 431) | |
| mmu-miR-93*- | ACUGCUGAGCUAGCACUUCCCG | MIMAT0004636 |
| 4395250 | (SEQ ID NO: 432) | |
| mmu-miR-99b*- | CAAGCUCGUGUCUGUGGGUCCG | MIMAT0004525 |
| 4395307 | (SEQ ID NO: 433) | |
| rno-miR-664- | UAUUCAUUUACUCCCCAGCCUA | MIMAT0003382 |
| 4381103 | (SEQ ID NO: 444) | |
| rno-miR-743a- | GAAAGACGCCAAACUGGGUAGA | MIMAT0005334 |
| 4395757 | (SEQ ID NO: 445) | |
| snoRNA135-4380912 | CTAAAATAGCTGGAATTACCGGC | Mature miRNA |
| | AGATTGGTAGTGGTGAGCCTATG | Control |
| | GTTTTCTGAAG (SEQ ID NO: 446) | |
| U87-4386735 | ACAATGATGACTTATGTTTTTGCC | Mature miRNA |
| | GTTTACCCACTCTGAGGGTTTCTTCT | Control |
| | GAAGAGAGAATCTTAAGACTGAG | |
| | C (SEQ ID NO: 447) | |

Table 11 lists miRNAs found in rat PC MVs and PCs, excluding exosomes.

TABLE 11
miRNAs found in rat MV and cells (excluding exosomes)

| miRNA in MVs | | Alternative |
|---|---|---|
| from Rat PDPCs | Exemplary Sequence(s) (5′ to 3′) | Description |
| mmu-miR-10a- | UACCCUGUAGAUCCGAAUUUGUG (SEQ ID | MIMAT0000648 |
| 437315 | NO: 448) | |
| mmu-miR-142-3p- | UGUAGUGUUUCCUACUUUAUGGA (SEQ ID | MIMAT0000155 |
| 4373136 | NO: 449) | |
| mmu-miR-146a- | UGAGAACUGAAUUCCAUGGGUU (SEQ ID | MIMAT0000158 |
| 4373132 | NO: 450) | |
| mmu-miR-148b- | UCAGUGCAUCACAGAACUUUGU (SEQ ID | MIMAT0000580 |
| 4373129 | NO: 451) | |
| mmu-miR-188-5p- | CAUCCCUUGCAUGGUGGAGGG (SEQ ID | MIMAT0000217 |
| 4395431 | NO: 452) | |
| mmu-miR-196b- | UAGGUAGUUUCCUGUUGUUGGG (SEQ ID | MIMAT0001081 |
| 4395326 | NO: 453) | |
| mmu-miR-204- | UUCCCUUUGUCAUCCUAUGCCU (SEQ ID | MIMAT0000237 |
| 4373094 | NO: 454) | |
| mmu-miR-210- | CUGUGCGUGUGACAGCGGCUGA (SEQ ID | MIMAT0000658 |
| 4373089 | NO: 455) | |
| mmu-miR-21- | UAGCUUAUCAGACUGAUGUUGA (SEQ ID | MIMAT0000530 |
| 4373090 | NO: 456) | |
| mmu-miR-23b- | AUCACAUUGCCAGGGAUUACC (SEQ ID | MIMAT0000125 |
| 4373073 | NO: 457) | |
| mmu-miR-26a- | UUCAAGUAAUCCAGGAUAGGCU (SEQ ID | MIMAT0000533 |
| 4395166 | NO: 458) | |
| mmu-miR-29c- | UAGCACCAUUUGAAAUCGGUUA (SEQ ID | MIMAT0000536 |
| 4395171 | NO: 459) | |
| mmu-miR-320- | AAAAGCUGGGUUGAGAGGGCGA (SEQ ID | MIMAT0000666 |
| 4395388 | NO: 460) | |
| mmu-miR-372- | CAGCAGCAAUUCAUGUUUUGGA (SEQ ID | MIMAT0000548 |
| 4378107 | NO: 461) | |
| mmu-miR-335-3p- | UUUUUCAUUAUUGCUCCUGACC (SEQ ID | MIMAT0004704 |
| 4395296 | NO: 462) | |
| mmu-miR-351- | UCCCUGAGGAGCCCUUUGAGCCUG (SEQ | MIMAT0000609 |
| 4373345 | ID NO: 463) | |
| mmu-miR-652- | AAUGGCGCCACUAGGGUUGUG (SEQ ID | MIMAT0003711 |
| 4395463 | NO: 464) | |
| mmu-miR-667- | UGACACCUGCCACCCAGCCCAAG (SEQ ID | MIMAT0003734 |
| 4386769 | NO: 465) | |
| mmu-miR-743b-5p- | UGUUCAGACUGGUGUCCAUCA (SEQ ID | MIMAT0004839 |
| 4395600 | NO: 466) | |
| rno-miR-351- | UCCCUGAGGAGCCCUUUGAGCCUG | MIMAT0000609 |
| 4395764 | (SEQ ID NO: 467) | |
| mmu-miR-130b*- | ACUCUUUCCCUGUUGCACUACU (SEQ ID | MIMAT0004583 |
| 4395590 | NO: 468) | |
| mmu-miR-135a*- | UAUAGGGAUUGGAGCCGUGGCG (SEQ ID | MIMAT0004531 |
| 4395343 | NO: 469) | |
| mmu-miR-149- | UCUGGCUCCGUGUCUUCACUCCC (SEQ ID | MIMAT0000159 |
| 4395366 | NO: 470) | |
| mmu-miR-206- | UGGAAUGUAAGGAAGUGUGUGG (SEQ ID | MIMAT0000239 |
| 4373092 | NO: 471) | |
| mmu-miR-26b*- | CCUGUUCUCCAUUACUUGGCUC (SEQ ID | MIMAT0004630 |
| 4395555 | NO: 472) | |
| mmu-miR-29b*- | GCUGGUUUCAUAUGGUGGUUUA (SEQ ID | MIMAT0004523 |
| 4395627 | NO: 473) | |
| mmu-miR-322*- | AAACAUGAAGCGCUGCAACAC (SEQ ID | MIMAT0000549 |
| 4395636 | NO: 474) | |
| mmu-miR-378- | ACUGGACUUGGAGUCAGAAGG (SEQ ID | MIMAT0003151 |
| 4395354 | NO: 475) | |
| mmu-miR-466d-3p- | UAUACAUACACGCACACAUAG (SEQ ID | MIMAT0004931 |
| 4395665 | NO: 476) | |
| mmu-miR-467b*- | AUAUACAUACACACACCAACAC (SEQ ID | MIMAT0003478 |
| 4381092 | NO: 477) | |
| mmu-miR-673-5p- | CUCACAGCUCUGGUCCUUGGAG (SEQ ID | MIMAT0003739 |
| 4386772 | NO: 478) | |
| mmu-miR-674*- | CACAGCUCCCAUCUCAGAACAA (SEQ ID | MIMAT0003741 |
| 4386773 | NO: 479) | |
| mmu-miR-678- | GUCUCGGUGCAAGGACUGGAGG (SEQ ID | MIMAT0003452 |
| 4381076 | NO: 480) | |
| mmu-miR-720- | AUCUCGCUGGGGCCUCCA | MIMAT0003484 |
| 4381052 | (SEQ ID NO: 481) | |
| mmu-miR-721- | CAGUGCAAUUAAAAGGGGGAA | MIMAT0003515 |
| 4381073 | (SEQ ID NO: 482) | |
| mmu-miR-760- | CGGCUCUGGGUCUGUGGGGA (SEQ ID | MIMAT0003898 |
| 4395439 | NO: 483) | |
| mmu-miR-801- | GAUUGCUGUGCGUGCGGAAUCGAC | |
| 4395562 | (SEQ ID NO: 484) | |
| mmu-miR-875-5p- | UAUACCUCAGUUUUAUCAGGUG (SEQ ID | MIMAT0004937 |
| 4395314 | NO: 485) | |
| mmu-miR-877- | GUAGAGGAGAUGGCGCAGGG (SEQ ID | MIMAT0004861 |
| 4395402 | NO: 486) | |
| mmu-miR-99b*- | CAAGCUCGUGUCUGUGGGUCCG (SEQ ID | MIMAT0004525 |
| 4395307 | NO: 487) | |
| rno-miR-463- | UGAUAGACGCCAAUUUGGGUAG | MIMAT0005317 |
| 4395751 | (SEQ ID NO: 488) | |
| snoRNA135- | CTAAAATAGCTGGAATTACCGGCAGATTGG | Mature miRNA |
| 4380912 | TAGTGGTGAGCCTATGGTTTTCTGAAG | Control |
| | (SEQ ID NO: 489) | |

Table 12 lists miRNAs found in rat exosomes and PCs, excluding extrasectetory vesicles larger than exosomes

TABLE 12
miRNAs found in rat PC04 exosomes and cells (excluding extrasecretory
vesicles larger than exosomes)

| miRNA in MVs | | Alternative |
|---|---|---|
| from Rat PCs | Exemplary Sequence(s) (5′ to 3′) | Description |
| mmu-miR-10a- | UACCCUGUAGAUCCGAAUUUGU (SEQ ID | MIMAT0000648 |
| 4373153 | NO: 434) | |
| mmu-miR-125a-5p- | UCCCUGAGACCCUUUAACCUGUGA (SEQ | MIMAT0000135 |
| 4395309 | ID NO: 435) | |
| mmu-miR-128a- | UCACAGUCAACCGGUCUCUUU | MIMAT0000424 |
| 4395327 | (SEQ ID NO: 436) | |
| mmu-miR-129-3p- | AAGCCCUUACCCCAAAAAGCAU (SEQ ID | MIMAT0000544 |
| 4373297 | NO: 437) | |
| mmu-miR-146a- | UGAGAACUGAAUUCCAUGGGUU (SEQ ID | MIMAT0000158 |
| 4373132 | NO: 438) | |
| mmu-miR-151-3p- | CUAGACUGAGGCUCCUUGAGG (SEQ ID | MIMAT0000161 |
| 4373304 | NO: 439) | |
| mmu-miR-187- | UCGUGUCUUGUGUUGCAGCCGG (SEQ ID | MIMAT0000216 |
| 4373307 | NO: 440) | |
| mmu-miR-188-5p- | CAUCCCUUGCAUGGUGGAGGG (SEQ ID | MIMAT0000217 |
| 4395431 | NO: 441) | |
| mmu-miR-197- | UUCACCACCUUCUCCACCCAGC | MIMAT0000227 |
| 4373102 | (SEQ ID NO: 442) | |
| mmu-miR-199a-3p- | ACAGUAGUCUGGACAUUGGUUA (SEQ ID | MIMAT0000230 |
| 4395415 | NO: 443) | |
| mmu-miR-204- | UUCCCUUUGUCAUCCUAUGCCU (SEQ ID | MIMAT0000237 |
| 4373094 | NO: 588) | |
| mmu-miR-210- | CUGUGCGUGUGACAGCGGCUGA (SEQ ID | MIMAT0000658 |
| 4373089 | NO: 589) | |
| mmu-miR-222- | AGCUACAUCUGGCUACUGGGU (SEQ ID | MIMAT0000670 |
| 4395387 | NO 590) | |
| mmu-miR-23b- | AUCACAUUGCCAGGGAUUACC (SEQ ID | MIMAT0000125 |
| 4373073 | NO: 591) | |
| mmu-miR-29c- | UAGCACCAUUUGAAAUCGGUUA (SEQ ID | MIMAT0000536 |
| 4395171 | NO: 592) | |
| mmu-miR-320- | AAAAGCUGGGUUGAGAGGGCGA (SEQ ID | MIMAT0000666 |
| 4395388 | NO: 593) | |
| mmu-miR-335-3p- | UUUUUCAUUAUUGCUCCUGACC (SEQ ID | MIMAT0004704 |
| 4395296 | NO: 594) | |
| mmu-miR-450a-5p- | UUUUGCGAUGUGUUCCUAAUAU (SEQ ID | MIMAT0001546 |
| 4395414 | NO: 595) | |
| mmu-miR-494- | UGAAACAUACACGGGAAACCUC (SEQ ID | MIMAT0003182 |
| 4395476 | NO: 596) | |
| mmu-miR-542-3p- | UGUGACAGAUUGAUAACUGAAA (SEQ ID | MIMAT0003172 |
| 4378101 | NO: 597) | |
| mmu-miR-652- | AAUGGCGCCACUAGGGUUGUG (SEQ ID | MIMAT0003711 |
| 4395463 | NO 598) | |
| mmu-miR-744- | UGCGGGGCUAGGGCUAACAGCA (SEQ ID | MIMAT0004187 |
| 4395435 | NO: 599) | |
| mmu-miR-190b- | UAGGUAGUUUCGUGUUGUUGGG | MIMAT0005303 |
| 4395749 | (SEQ ID NO: 600) | |
| mmu-miR-135a*- | UAUAGGGAUUGGAGCCGUGGCG (SEQ ID | MIMAT0004531 |
| 4395343 | NO 601) | |
| mmu-miR-18a*- | ACUGCCCUAAGUGCUCCUUCUG (SEQ ID | MIMAT0004626 |
| 4395620 | NO: 602) | |
| mmu-miR-206- | UGGAAUGUAAGGAAGUGUGUGG (SEQ ID | MIMAT0000239 |
| 4373092 | NO: 603) | |
| mmu-miR-214*- | UGCCUGUCUACACUUGCUGUGC (SEQ ID | MIMAT0004664 |
| 4395404 | NO: 604) | |
| mmu-miR-26b*- | CCUGUUCUCCAUUACUUGGCUC (SEQ ID | MIMAT0004630 |
| 4395555 | NO: 605) | |
| mmu-miR-29b*- | GCUGGUUUCAUAUGGUGGUUUA (SEQ ID | MIMAT0004523 |
| 4395627 | NO: 606) | |
| mmu-miR-30e*- | CUUUCAGUCGGAUGUUUACAGC (SEQ ID | MIMAT0000249 |
| 4373057 | NO: 607) | |
| mmu-miR-326- | CCUCUGGGCCCUUCCUCCAGU (SEQ ID | MIMAT0000559 |
| 4373335 | NO: 608) | |
| mmu-miR-378- | 4ACUGGACUUGGAGUCAGAAGG (SEQ ID | MIMAT0003151 |
| 395354 | NO 609) | |
| mmu-miR-466d-3p- | UAUACAUACACGCACACAUAG (SEQ ID | MIMAT0004931 |
| 4395665 | NO: 610) | |
| mmu-miR-467b*- | AUAUACAUACACACACCAACAC (SEQ ID | MIMAT0003478 |
| 4381092 | NO: 611) | |
| mmu-miR-674*- | CACAGCUCCCAUCUCAGAACAA (SEQ ID | MIMAT0003741 |
| 4386773 | NO: 612) | |
| mmu-miR-720- | AUCUCGCUGGGGCCUCCA | MIMAT0003484 |
| 4381052 | (SEQ ID NO: 613) | |
| mmu-miR-721- | CAGUGCAAUUAAAAGGGGGAA | MIMAT0003515 |
| 4381073 | (SEQ ID NO: 614) | |
| mmu-miR-801- | GAUUGCUGUGCGUGCGGAAUCGAC | |
| 4395562 | (SEQ ID NO: 615) | |
| mmu-miR-877- | GUAGAGGAGAUGGCGCAGGG (SEQ ID | MIMAT0004861 |
| 4395402 | NO: 616) | |
| mmu-miR-9*- | AUAAAGGUAGAUAACCGAAAGU (SEQ ID | MIMAT0000143 |
| 4395342 | NO: 617) | |
| mmu-miR-99b*- | CAAGCUCGUGUCUGUGGGUCCG (SEQ ID | MIMAT0004525 |
| 4395307 | NO: 490) | |
| snoRNA135-4380912 | CTAAAATAGCTGGAATTACCGGCAGATTGG | Mature miRNA |
| | TAGTGGTGAGCCTATGGTTTTCTGAAG | Control |
| | (SEQ ID NO: 491) | |

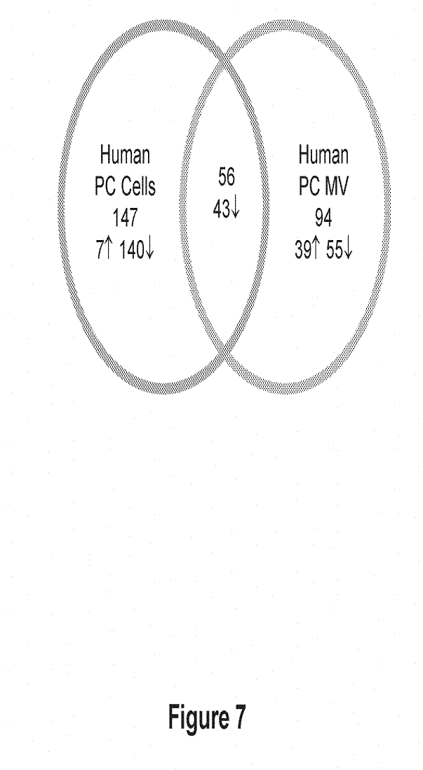

microRNA expression profiles for human PCs and MVs obtained from human PCs grown under serum starvation conditions were determined and compared. As shown in FIG. 7, microRNA whose expression was altered by growth under serum starvation conditions for 24 hours as compared with growth under serum replete conditions was determined and overlapping microRNA sequences among human PCs and MVs were identified. As can be seen in FIG. 7, there were 43 miRNAs in common to all samples which had decreased expression in response to serum starvation.

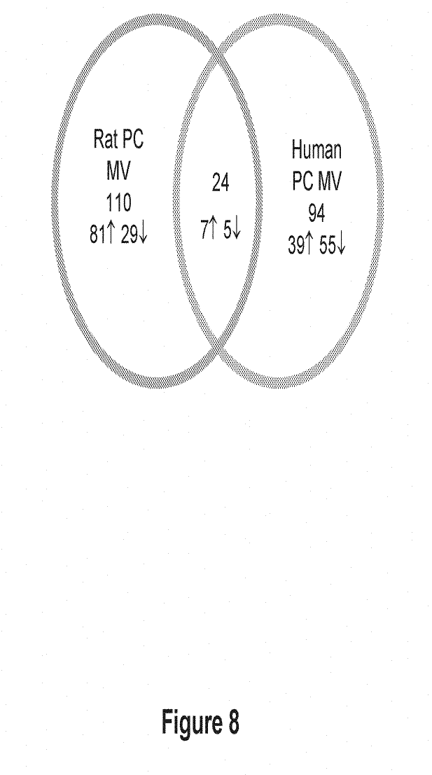

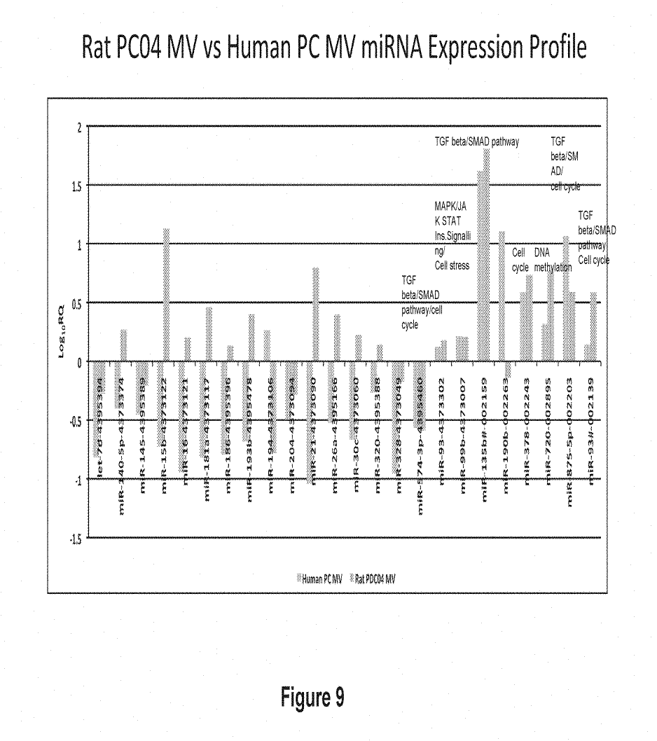

miRNAs from MVs obtained from human PCs grown under serum starvation conditions were compared to those obtained from rat PCs grown under comparable conditions. As can be seen in FIG. 8, there were 7 miRNAs in common that had increased expression in response to serum starvation. FIG. 9 shows an exemplary graph comparison of miRNA expression profiles for rat MVs and human MVs obtained from PCs grown under serum starvation conditions. As can be seen in FIG. 9, microRNAs whose expression was increased in response to serum starvation may play roles in various cellular functions, including cell cycle, MAPK, signaling pathways, TGF beta signaling pathways, and DNA methylation, among others.

Table 13 depicts results from analysis of miRNAs from MVs obtained from human PC RNA preparations.

TABLE 13
miRNAs from MVs obtained from human PCs grown under serum starvation conditions

| miRNA in | | |
|---|---|---|
| MVs from | | Alternative |
| Human PCs | Exemplary Sequence(s) (5′ to 3′) | Description |
| has-miR-155- | UUAAUGCUAAUCGUGAUAGGGGU (SEQ ID | MIMAT0000646 |
| 4395459 | NO: 492) | |
| hsa-let-7b- | UGAGGUAGUAGGUUGUGUGGUU (SEQ ID | MIMAT0000063 |
| 4395446 | NO: 493) | |
| hsa-let-7d- | AGAGGUAGUAGGUUGCAUAGUU (SEQ ID | MIMAT0000065 |
| 4395394 | NO: 494) | |
| hsa-let-7e- | UGAGGUAGGAGGUUGUAUAGUU (SEQ ID | MIMAT0000066 |
| 4395517 | NO: 495) | |
| hsa-miR-100- | AACCCGUAGAUCCGAACUUGUG (SEQ ID | MIMAT0000098 |
| 4373160 | NO: 496) | |
| hsa-miR-125a- | UCCCUGAGACCCUUUAACCUGUGA (SEQ ID | MIMAT0000443 |
| 5p-4395309 | NO: 497) | |
| hsa-miR-125b- | UCCCUGAGACCCUAACUUGUGA (SEQ ID | MIMAT0000423 |
| 4373148 | NO: 498) | |
| hsa-miR-126- | UCGUACCGUGAGUAAUAAUGCG (SEQ ID | MIMAT0000445 |
| 4395339 | NO: 499) | |
| hsa-miR-134- | UGUGAGUGGUUGACCAGAGGGG (SEQ ID | MIMAT0000447 |
| 4373299 | NO: 500) | |
| hsa-miR-138- | AGCUGGUGUUGUGAAUCAGGCCG (SEQ ID | MIMAT0000430 |
| 4395395 | NO: 501) | |
| hsa-miR-139- | UCUACAGUGCACGUGUCUCCAG (SEQ ID | MIMAT0000250 |
| 52-4395400 | NO: 502) | |
| hsa-miR-140- | CAGUGGUUUUACCCUAUGGUAG (SEQ ID | MIMAT0000431 |
| 5p-4373374 | NO: 503) | |
| hsa-miR-143- | UGAGAUGAAGCACUGUAGCUC (SEQ ID | MIMAT0000435 |
| 4395360 | NO: 504) | |
| hsa-miR-145- | GUCCAGUUUUCCCAGGAAUCCCU (SEQ ID | MIMAT0000437 |
| 4395389 | NO: 505) | |
| hsa-miR-149- | UCUGGCUCCGUGUCUUCACUCCC (SEQ ID | MIMAT0000450 |
| 4395366 | NO: 506) | |
| hsa-miR-152- | UCAGUGCAUGACAGAACUUGG (SEQ ID | MIMAT0000438 |
| 4395170 | NO: 507) | |
| ha-miR-153- | UUGCAUAGUCACAAAAGUGAUC (SEQ ID | MIMAT0000439 |
| 4373305 | NO: 508) | |
| hsa-miR-15b- | UAGCAGCACAUCAUGGUUUACA (SEQ ID | MIMAT0000417 |
| 4373122 | NO: 509) | |
| hsa-miR-16- | UAGCAGCACGUAAAUAUUGGCG (SEQ ID | MIMAT0000069 |
| 4373121 | NO: 510) | |
| hsa-miR-17- | CAAAGUGCUUACAGUGCAGGUAG (SEQ ID | MIMAT0000070 |
| 4395419 | NO: 511) | |
| hsa-miR-181a- | AACAUUCAACGCUGUCGGUGAGU (SEQ ID | MIMAT0000256 |
| 4373117 | NO: 512) | |
| hsa-miR-184- | UGGACGGAGAACUGAUAAGGGU (SEQ ID | MIMAT0000454 |
| 4373113 | NO: 513) | |
| hsa-miR-186- | CAAAGAAUUCUCCUUUUGGGU (SEQ ID | MIMAT0000456 |
| 4395396 | NO: 514) | |
| hsa-miR-191- | CAACGGAAUCCCAAAAGCAGCUG (SEQ ID | MIMAT0000440 |
| 4395410 | NO: 515) | |
| hsa-miR-193b- | AACUGGCCCUCAAAGUCCCGCU (SEQ ID | MIMAT0002819 |
| 4395478 | NO: 516) | |
| hsa-miR-194- | UGUAACAGCAACUCCAUGUGGA (SEQ ID | MIMAT0000460 |
| 4373106 | NO: 517) | |
| hsa-miR-197- | UUCACCACCUUCUCCACCCAGC (SEQ ID | MIMAT0000227 |
| 4373102 | NO: 518) | |
| hsa-miR-199a- | ACAGUAGUCUGCACAUUGGUUA (SEQ ID | MIMAT0000232 |
| 3p-4395415 | NO: 519) | |
| hsa-miR-19b- | UGUGCAAAUCCAUGCAAAACUGA (SEQ ID | MIMAT0000074 |
| 4373098 | NO: 520) | |
| hsa-miR-204- | UUCCCUUUGUCAUCCUAUGCCU (SEQ ID | MIMAT0000265 |
| 4373094 | NO: 521) | |
| hsa-miR-208- | AUAAGACGAGCAAAAAGCUUGU (SEQ ID | |
| 4373091 | NO: 522) | |
| hsa-miR-212- | UAACAGUCUCCAGUCACGGCC (SEQ ID | MIMAT0000269 |
| 4373087 | NO: 523) | |
| hsa-miR-21- | UAGCUUAUCAGACUGAUUGUUGA SEQ ID | MIMAT0000076 |
| 4373090 | NO: 524) | |
| hsa-miR-221- | AGCUACAUUGUCUGCUGGGUUUC (SEQ ID | MIMAT0000278 |
| 4373077 | NO: 525) | |
| hsa-miR-222- | AGCUACAUCUGGCUACUGGGU (SEQ ID | MIMAT0000279 |
| 4395387 | NO: 526) | |
| hsa-miR-223- | UGUCAGUUUGUCAAAUACCCCA (SEQ ID | MIMAT0000280 |
| 4395406 | NO: 527) | |
| hsa-miR-26a- | UUCAAGUAAUCCAGGAUAGGCU (SEQ ID | MIMAT0000082 |
| 4395166 | NO: 528) | |
| hsa-miR-27a- | UUCACAGUGGCUAAGUUCCGC (SEQ ID | MIMAT0000084 |
| 4373287 | NO: 529) | |
| hsa-miR-28- | CACUAGAUUGUGAGCUCCUGGA (SEQ ID | MIMAT0004502 |
| 3p-4395557 | NO: 530) | |
| ha-miR-29a- | UAGCACCAUCUGAAAUCGGUUA (SEQ ID | MIMAT0000086 |
| 4395223 | NO: 531) | |
| hsa-miR-302a- | UAAGUGCUUCCAUGUUUUGGUGA (SEQ ID | MIMAT0000684 |
| 4378070 | NO: 532) | |
| hsa-miR-302b- | UAAGUGCUUCCAUGUUUUAGUAG (SEQ ID | MIMAT0000715 |
| 4378071 | NO: 533) | |
| hsa-miR-30b- | UGUAAACAUCCUACACUCAGCU (SEQ ID | MIMAT0000420 |
| 4373290 | NO: 534) | |
| hsa-miR-30c- | UGUAAACAUCCUACACUCUCAGC (SEQ ID | MIMAT0000244 |
| 4373060 | NO: 535) | |
| hsa-miR-31- | AGGCAAGAUGCUGGCAUAGCU (SEQ ID | MIMAT0000089 |
| 4395390 | NO: 536) | |
| hsa-miR-320- | AAAAGCUGGGUUGAGAGGGCGA (SEQ ID | MIMAT0000510 |
| 4395388 | NO: 537) | |
| hsa-miR-323- | CACAUUACACGGUCGACCUCU (SEQ ID | MIMAT0000755 |
| 32-4395338 | NO: 538) | |
| hsa-miR-328- | CUGGCCCUCUCUGCCCUUCCGU (SEQ ID | MIMAT0000752 |
| 4373049 | NO: 539) | |
| hsa-miR-342- | UCUCACACAGAAAUCGCACCCGU (SEQ ID | MIMAT0000753 |
| 3p-4395371 | NO: 540) | |
| hsa-miR-365- | UAAUGCCCCUAAAAAUCCUUAU (SEQ ID | MIMAT0000710 |
| 4373194 | NO: 541) | |
| hsa-miR- | UUAUAAUACAACCUGAUAAGUG (SEQ ID | MIMAT0000727 |
| 374a-4373028 | NO: 542) | |
| hsa-miR- | AUCAUAGAGGAAAAUCCACGU (SEQ ID | MIMAT0000729 |
| 376a-4373026 | NO: 543) | |
| hsa-miR- | AACAUAGAGGAAAUUCCACGU (SEQ ID | MIMAT0000720 |
| 376c-4395233 | NO: 544) | |
| hsa-miR-454- | UAGUGCAAUAUUGCUUAUAGGGU (SEQ ID | MIMAT0003885 |
| 4395434 | NO: 545) | |
| hsa-miR-483- | AAGACGGGAGGAAAGAAGGGAG (SEQ ID | MIMAT0004761 |
| 52-4395449 | NO: 546) | |
| hsa-miR-491- | AGUGGGGAACCCUUCCAUGAGG (SEQ ID | MIMAT0002807 |
| 5p-4381053 | NO: 547) | |
| hsa-miR-518d- | CAAAGCGCUUCCCUUUGGAGC (SEQ ID | MIMAT0002864 |
| 3p-4373248 | NO: 548) | |
| hsa-miR- | GAAAGCGCUUCUCUUUAGAGG (SEQ ID | MIMAT0002842 |
| 518f-4395499 | NO: 549) | |
| hsa-miR-523- | GAACGCGCUUCCCUAUAGAGGGU (SEQ ID | MIMAT0002840 |
| 4395497 | NO: 550) | |
| hsa-miR-532- | CAUGCCUUGAGUGUAGGACCGU (SEQ ID | MIMAT0002888 |
| 5p-4380928 | NO: 551) | |
| hsa-miR-574- | CACGCUCAUGCACACACCCACA (SEQ ID | MIMAT0003239 |
| 32-4395460 | NO: 552) | |
| hsa-miR-618- | AAACUCUACUUGUCCUUCUGAGU (SEQ ID | MIMAT0003287 |
| 4380996 | NO: 553) | |
| hsa-miR-636- | UGUGCUUGCUCGUCCCGCCCGCA (SEQ ID | MIMAT0003306 |
| 4395199 | NO: 554) | |
| hsa-miR-93- | CAAAGUGCUGUUCGUGCAGGUAG (SEQ ID | MIMAT0000093 |
| 4373302 | NO: 555) | |
| hsa-miR-99b- | CACCCGUAGAACCGACCUUGCG (SEQ ID | MIMAT0000689 |
| 4373007 | NO: 556) | |
| RNU48- | GATGACCCCAGGTAACTCTGAGTGTGTCGC | Mature miRNA |
| 4373383 | TGATGCCATCACCGCAGCGCTCTGACC (SEQ | Control |
| | ID NO: 557) | |
| has-miR- | UUUUCAACUCUAAUGGGAGAGA (SEQ ID | |
| 1305-002867 | NO: 558) | |
| hsa-miR- | UCACUGUUCAGACAGGCGGA (SEQ ID | MIMAT0005873 |
| 1208-002880 | NO: 559) | |
| hsa-miR- | CGGAUGAGCAAAGAAAGUGGUU (SEQ ID | MIMAT0005945 |
| 1243-002854 | NO: 560) | |
| hsa-miR-1255B- | CGGAUGAGCAAAGAAAGUGGUU (SEQ ID | MIMAT0005945 |
| 002801 | NO: 561) | |
| hsa-miR- | AUGGGUGAAUUUGUAGAAGGAU (SEQ ID | MIMAT0005914 |
| 1262-002852 | NO: 562) | |
| hsa-miR-1274A- | GUCCCUGUUCAGGCGCCA | |
| 002883 | (SEQ ID NO: 563) | |
| hsa-miR-127413- | UCCCUGUUCGGGCGCCA | MIMAT0005938 |
| 002884 | (SEQ ID NO: 564) | |
| hsa-miR- | UUCAUUCGGCUGUCCAGAUGUA (SEQ ID | MIMAT0005800 |
| 1298-002861 | NO: 565) | |
| hsa-miR- | UAUGGCUUUUCAUUCCUAUGUGA (SEQ ID | MIMAT0000758 |
| 135b#-002159 | NO: 566) | |
| hsa-miR-144- | UACAGUAUAGAUGAUGUACU (SEQ ID | MIMAT0000436 |
| 002676 | NO: 567) | |
| hsa-miR-151- | CUAGACUGAAGCUCCUUGAGG (SEQ ID | MIMAT0000757 |
| 3p-002254 | NO: 568) | |
| hsa-miR- | UGAUAUGUUUGAUAUUGGGUU (SEQ ID | MIMAT0004929 |
| 190b-002263 | NO: 569) | |
| hsa-miR-19b- | UGUGCAAAUCCAUGCAAAACUGA (SEQ ID | MIMAT0000074 |
| 1#-002425 | NO: 370) | |
| hsa-miR-21#- | UAGCUUAUCAGACUGAUGUUGA (SEQ ID | MIMAT0000076 |
| 002438 | NO: 571) | |
| hsa-miR-30e- | CUUUCAGUCGGAUGUUUACAGC (SEQ ID | MIMAT0000693 |
| 3p-000422 | NO: 572) | |
| hsa-miR- | UCAAGAGCAAUAACGAAAAAUGU (SEQ ID | MIMAT0000765 |
| 335#-002185 | NO: 573) | |
| hsa-miR- | UGGCAGUGUCUUAGCUGGUUGU (SEQ ID | MIMAT0000255 |
| 34a#-002316 | NO: 574) | |
| hsa-miR-378- | ACUGGACUUGGAGUCAGAAGG (SEQ ID | MIMAT0000732 |
| 002243 | NO: 575) | |
| hsa-miR- | AAAGUGCUUCCUUUUAGAGGGU (SEQ ID | MIMAT0002846 |
| 520c-3p- | NO: 576) | |
| 002400 | | |
| hsa-miR-571- | UGAGUUGGCCAUCUGAGUGAG (SEQ ID | MIMAT0003236 |
| 001613 | NO: 577) | |
| hsa-miR-601- | UGGUCUAGGAUUGUUGGAGGAG (SEQ ID | MIMAT0003269 |
| 001558 | NO: 578) | |
| hsa-miR- | AGGGGGAAAGUUCUAUAGUCC (SEQ ID | MIMAT0003294 |
| 625#-002432 | NO: 579) | |
| hsa-miR-639- | AUCGCUGCGGUUGCGAGCGCUGU (SEQ ID | MIMAT0003309 |
| 001583 | NO: 580) | |
| hsa-miR-643- | ACUUGUAUGCUAGCUCAGGUAG (SEQ ID | MIMAT0003313 |
| 001594 | NO: 581) | |
| hsa-miR-720- | UCUCGCUGGGGCCUCCA | MIMAT0005954 |
| 002895 | (SEQ ID NO: 582) | |
| hsa-miR-767- | UCUGCUCAUACCCCAUGGUUUCU (SEQ ID | MIMAT0003883 |
| 3p-001995 | NO: 583) | |
| hsa-miR-875- | UAUACCUCAGUUUUAUCAGGUG (SEQ ID | MIMAT0004922 |
| 5p-002203 | NO: 584) | |
| hsa-miR- | CACUGGCUCCUUUCUGGGUAGA (SEQ ID | MIMAT0004918 |
| 892b-002214 | NO: 585) | |
| hsa-miR-93#- | CAAAGUGCUGUUCGUGCAGGUAG (SEQ ID | MIMAT0000093 |
| 002139 | NO: 586) | |
| RNU48- | GATGACCCCAGGTAACTCTGAGTGTGTCGC | Mature miRNA |
| 001006 | TGATGCCATCACCGCAGCGCTCTGACC (SEQ | Control |
| | ID NO: 587) | |

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any method of use; etc.) can be excluded from any one or more claims, for any reason, whether or not related to the existence of prior art.

### INCORPORATION OF REFERENCES

All publications and patent documents cited in this application are incorporated by reference in their entirety to the same extent as though the contents of each individual publication or patent document were incorporated herein.

What is claimed is:

1. A composition comprising purified microvesicles derived from pathfinder cells.

2. The composition of claim 1, wherein the pathfinder cells are derived from pancreas, kidney, liver, spleen, lymph node, myometrium, peripheral blood, cord blood, bone marrow, serum, mesenchymal tissue, or a combination thereof.

3. (canceled)

4. The composition of claim 1, wherein the pathfinder cells are mammalian.

5. The composition of claim 4, wherein the mammalian cells are rat.

6. The composition of claim 4, wherein the mammalian cells are human.

7-9. (canceled)

10. The composition of claim 1, wherein the microvesicles have a mean diameter of from about 100 nm to about 1000 nm.

11. The composition of claim 1, wherein the pathfinder cells are cultured under hypoxic conditions.

12. The composition of claim 1, wherein the pathfinder cells are cultured in a medium that is substantially free of serum.

13. The composition of claim 1, wherein the microvesicles are characterized by an ability to increase the proliferation of cells.

14. The composition of claim 13, wherein the microvesicles are characterized by an ability to increase the proliferation of cells in an in vitro culture system.

15. The composition of claim 14, wherein the proliferation of cells is measured by doubling time.

16. The composition of claim 1, wherein the microvesicles are characterized by an ability to stimulate migration or regrowth of cells.

17. A method for increasing the proliferation of cells comprising the step of contacting the cells with a composition comprising purified microvesicles derived from pathfinder cells.

18. The method of claim 17, wherein the wherein the pathfinder cells are derived from pancreas, kidney, or lymph node pancreas, kidney, liver, spleen, lymph node, myometrium, peripheral blood, cord blood, bone marrow, serum, mesenchymal tissue, or a combination thereof.

19-21. (canceled)

22. The method of claim 17, wherein the microvesicles are administered in vivo.

23. The method of claim 17, wherein the microvesicles are administered in vitro.

24. A method of treating a disease, disorder, or condition associated with tissue damage in a subject comprising the step of administering to the subject a composition comprising purified microvesicles derived from pathfinder cells.

25. The method of claim 24, wherein the pathfinder cells are derived from pancreas, kidney, liver, spleen, lymph node, myometrium, peripheral blood, cord blood, bone marrow, serum, mesenchymal tissue, or a combination thereof.

26. The method of claim 24, wherein the disease, disorder, or condition is selected from the group consisting of diabetes mellitus, congestive myocardial failure, myocardial infarct, acute renal disease, chronic renal disease, and traumatic injury.

27. A method of preparing a purified population of microvesicles derived from pathfinder cells comprising steps of:
a. centrifuging the pathfinder cells one or more times at a centrifugal force of approximately 120,000 g or less to produce a pellet; and
b. harvesting microvesicles from the pellet.

28-30. (canceled)


SEQUENCE LISTING (617 sequences)

SEQ ID NO: 1 - RNA, 22 nt, Homo sapiens
uggagugugacaaugguguuug

SEQ ID NO: 2 - RNA, 22 nt, Rattus norvegicus
uggagugugacaaugguguuug

SEQ ID NO: 3 - RNA, 22 nt, Homo sapiens
cugaagcucagagggcucugau

SEQ ID NO: 4 - RNA, 22 nt, Homo sapiens
ucggauccgucugagcuuggcu

SEQ ID NO: 5 - RNA, 22 nt, Rattus norvegicus
ucggauccgucugagcuuggcu

SEQ ID NO: 6 - RNA, 22 nt, Homo sapiens
uuugguccccuucaaccagcua

SEQ ID NO: 7 - RNA, 22 nt, Rattus novergicus
uuugguccccuucaaccagcua

SEQ ID NO: 8 - RNA, 22 nt, Homo sapiens
aggugguccguggcgcguucgc

SEQ ID NO: 9 - RNA, 21 nt, Homo sapiens
cacauuacacggucgaccucu

SEQ ID NO: 10 - RNA, 21 nt, Rattus novergicus
cacauuacacggucgaccucu

SEQ ID NO: 11 - RNA, 22 nt, Rattus novergicus
aggugguccguggcgcguucgc

SEQ ID NO: 12 - RNA, 23 nt, Homo sapiens
ugucugcccgcaugccugccucu

SEQ ID NO: 13 - RNA, 23 nt, Rattus novergicus
ugucugccugagugccugccucu

SEQ ID NO: 14 - RNA, 22 nt, Homo sapiens
aucaugaugggcuccucggugu

SEQ ID NO: 15 - RNA, 22 nt, Rattus norvegicus
aucaugaugggcuccucggugu

SEQ ID NO: 16 - RNA, 22 nt, Homo sapiens
aaaccguuaccauuacugaguu

SEQ ID NO: 17 - RNA, 22 nt, Rattus norvegicus
aaaccguuaccauuacugaguu

SEQ ID NO: 18 - RNA, 22 nt, Mus musculus
ugugugcaugugcuugugugua

SEQ ID NO: 19 - RNA, 22 nt, Mus musculus
uaagugcgugcauguauaugug

SEQ ID NO: 20 - RNA, 22 nt, Mus musculus
auaagugugagcauguauaugu

SEQ ID NO: 21 - RNA, 22 nt, Mus musculus
auauacauacacacaccuauau

SEQ ID NO: 22 - RNA, 23 nt, Mus musculus
uaugacugaugugcgugugucug

SEQ ID NO: 23 - RNA, 22 nt, Homo sapiens
aguggggaacccuuccaugagg

SEQ ID NO: 24 - RNA, 22 nt, Homo sapiens
cuuaugcaagauucccuucuac

SEQ ID NO: 25 - RNA, 22 nt, Homo sapiens
aaacaaacauggugcacuucuu

SEQ ID NO: 26 - RNA, 22 nt, Rattus norvegicus
aaacaaacauggugcacuucuu

SEQ ID NO: 27 - RNA, 16 nt, Mus musculus
augguggcacggaguc

SEQ ID NO: 28 - RNA, 22 nt, Rattus norvegicus
agcgggcacggcugugagagcc

SEQ ID NO: 29 - RNA, 21 nt, Mus musculus
gggcaucugcugacauggggg

SEQ ID NO: 30 - RNA, 23 nt, Homo sapiens
acuccauuuguuuugaugaugga

SEQ ID NO: 31 - RNA, 22 nt, Homo sapiens
caucaucgucucaaaugagucu

SEQ ID NO: 32 - RNA, 20 nt, Homo sapiens
agagguauagggcaugggaa

SEQ ID NO: 33 - RNA, 22 nt, Homo sapiens
uuccuaugcauauacuucuuug

SEQ ID NO: 34 - RNA, 21 nt, Rattus norvegicus
uuccuaugcauauacuucuuu

SEQ ID NO: 35 - RNA, 22 nt, Homo sapiens
uggaauguaaggaagugugugg

SEQ ID NO: 36 - RNA, 22 nt, Rattus norvegicus
uggaauguaaggaagugugugg

SEQ ID NO: 37 - RNA, 21 nt, Homo sapiens
caagucacuagugguuccguu

SEQ ID NO: 38 - RNA, 23 nt, Homo sapiens
aaaauggugcccuagugacuaca

SEQ ID NO: 39 - RNA, 23 nt, Rattus norvegicus
caagucacuagugguuccguuua

SEQ ID NO: 40 - RNA, 19 nt, Rattus norvegicus
ccuugaggggcaugagggu

SEQ ID NO: 41 - RNA, 19 nt, Rattus norvegicus
ugucccucugggucgccca

SEQ ID NO: 42 - RNA, 22 nt, Homo sapiens
agaucgaccguguuauauucgc

SEQ ID NO: 43 - RNA, 21 nt, Homo sapiens
aauaauacaugguugaucuuu

SEQ ID NO: 44 - RNA, 22 nt, Rattus norvegicus
agaucgaccguguuauauucgc

SEQ ID NO: 45 - RNA, 21 nt, Rattus norvegicus
aauaauacaugguugaucuuu

SEQ ID NO: 46 - RNA, 22 nt, Homo sapiens
gccugcugggguggaaccuggu

SEQ ID NO: 47 - RNA, 23 nt, Rattus norvegicus
gccugcugggguggaaccugguu

SEQ ID NO: 48 - RNA, 22 nt, Homo sapiens
uuuguucguucggcucgcguga

SEQ ID NO: 49 - RNA, 22 nt, Rattus norvegicus
uuuguucguucggcucgcguga

SEQ ID NO: 50 - RNA, 22 nt, Homo sapiens
aucauagaggaaaauccauguu

SEQ ID NO: 51 - RNA, 22 nt, Rattus norvegicus
guggauauuccuucuaugguua

SEQ ID NO: 52 - RNA, 21 nt, Rattus norvegicus
aucauagaggaacauccacuu

SEQ ID NO: 53 - RNA, 22 nt, Homo sapiens
uauacaagggcaagcucucugu

SEQ ID NO: 54 - RNA, 19 nt, Rattus norvegicus
uauacaagggcaagcucuc

SEQ ID NO: 55 - RNA, 22 nt, Rattus norvegicus
uuugaaccaucacucgacuccu

SEQ ID NO: 56 - RNA, 22 nt, Homo sapiens
aacuguuugcagaggaaacuga

SEQ ID NO: 57 - RNA, 22 nt, Homo sapiens
cucaucugcaaagaaguaagug

SEQ ID NO: 58 - RNA, 23 nt, Mus musculus
uauuuagaauggcacugauguga

SEQ ID NO: 59 - RNA, 22 nt, Mus musculus
gaucagggccuuucuaaguaga

SEQ ID NO: 60 - RNA, 22 nt, Mus musculus
uauuuagaauggugcugaucug

SEQ ID NO: 61 - RNA, 22 nt, Mus musculus
gaucagggccuuucuaaguaga

SEQ ID NO: 62 - RNA, 23 nt, Mus musculus
uucuuggacuggcacuggugagu

SEQ ID NO: 63 - RNA, 22 nt, Mus musculus
aaccaguaccuuucugagaaga

SEQ ID NO: 64 - RNA, 22 nt, Homo sapiens
aaucauacagggacauccaguu

SEQ ID NO: 65 - RNA, 22 nt, Homo sapiens
aaacauucgcggugcacuucuu

SEQ ID NO: 66 - RNA, 22 nt, Rattus norvegicus
aaguugcccgcguguuuuucgc

SEQ ID NO: 67 - RNA, 21 nt, Rattus norvegicus
aaacauucgcggugcacuucu

SEQ ID NO: 68 - RNA, 20 nt, Rattus norvegicus
uugguacuucuuuaagugag

SEQ ID NO: 69 - RNA, 22 nt, Homo sapiens
gagcuuauucauaaaagugcag

SEQ ID NO: 70 - RNA, 21 nt, Homo sapiens
uaauuuuauguauaagcuagu

SEQ ID NO: 71 - RNA, 23 nt, Mus musculus
ugagagaugccauucuauguaga

SEQ ID NO: 72 - RNA, 22 nt, Rattus norvegicus
aacuguggcauuucugaauaga

SEQ ID NO: 73 - RNA, 23 nt, Rattus norvegicus
uggaagacuugugauuuuguugu

SEQ ID NO: 74 - RNA, 22 nt, Rattus norvegicus
acugcagugaaggcacuugugg

SEQ ID NO: 75 - RNA, 22 nt, Rattus norvegicus
uauugcacauuacuaaguugca

SEQ ID NO: 76 - RNA, 23 nt, Rattus norvegicus
aggcaguguaguuagcugauugc

SEQ ID NO: 77 - RNA, 22 nt, Rattus norvegicus
aaucacuaaccacacagccagg

SEQ ID NO: 78 - RNA, 21 nt, Rattus norvegicus
cuuuuugcggucugggcuugc

SEQ ID NO: 79 - RNA, 22 nt, Rattus norvegicus
ugauauguuugauauauuaggu

SEQ ID NO: 80 - RNA, 22 nt, Rattus norvegicus
gugaaauguuuaggaccacuag

SEQ ID NO: 81 - RNA, 21 nt, Rattus norvegicus
aacauagaggaaauuucacgu

SEQ ID NO: 82 - RNA, 19 nt, Rattus norvegicus
uauacaagggcaagcucuc

SEQ ID NO: 83 - RNA, 22 nt, Rattus norvegicus
auuccuagaaauuguucacaau

SEQ ID NO: 84 - RNA, 22 nt, Rattus norvegicus
uaugugccuuuggacuacaucg

SEQ ID NO: 85 - RNA, 21 nt, Rattus norvegicus
uuaagacuugcagugauguuu

SEQ ID NO: 86 - RNA, 20 nt, Rattus norvegicus
gucaacacuugcugguuucc

SEQ ID NO: 87 - RNA, 22 nt, Rattus norvegicus
uacaguuguucaaccaguuacu

SEQ ID NO: 88 - RNA, 22 nt, Rattus norvegicus
uccgagccugggucucccucuu

SEQ ID NO: 89 - RNA, 22 nt, Rattus norvegicus
ggggguccccggugcucggauc

SEQ ID NO: 90 - RNA, 22 nt, Rattus norvegicus
agagguaguagguugcauaguu

SEQ ID NO: 91 - RNA, 23 nt, Rattus norvegicus
caaagugcuaacagugcagguag

SEQ ID NO: 92 - RNA, 21 nt, Rattus norvegicus
uaaagugcugacagugcagau

SEQ ID NO: 93 - RNA, 23 nt, Rattus norvegicus
uacccuguagauccgaauuugug

SEQ ID NO: 94 - RNA, 22 nt, Rattus norvegicus
ucguaccgugaguaauaaugcg

SEQ ID NO: 95 - RNA, 22 nt, Rattus norvegicus
cagugcaauguuaaaagggcau

SEQ ID NO: 96 - RNA, 22 nt, Rattus norvegicus
cagugcaaugaugaaagggcau

SEQ ID NO: 97 - RNA, 22 nt, Rattus norvegicus
cagugguuuuacccuaugguag

SEQ ID NO: 98 - RNA, 23 nt, Rattus norvegicus
uguaguguuuccuacuuuaugga

SEQ ID NO: 99 - RNA, 23 nt, Rattus norvegicus
guccaguuuucccaggaaucccu

SEQ ID NO: 100 - RNA, 22 nt, Rattus norvegicus
ugagaacugaauuccauggguu

SEQ ID NO: 101 - RNA, 22 nt, Rattus norvegicus
ugagaacugaauuccauaggcu

SEQ ID NO: 102 - RNA, 22 nt, Rattus norvegicus
ucagugcaucacagaacuuugu

SEQ ID NO: 103 - RNA, 23 nt, Rattus norvegicus
uuaaugcuaauugugauaggggu

SEQ ID NO: 104 - RNA, 22 nt, Rattus norvegicus
uagcagcacauaaugguuugug

SEQ ID NO: 105 - RNA, 22 nt, Rattus norvegicus
uagcagcacaucaugguuuaca

SEQ ID NO: 106 - RNA, 22 nt, Rattus norvegicus
uagcagcacguaaauauuggcg

SEQ ID NO: 107 - RNA, 23 nt, Rattus norvegicus
aacauucaacgcugucggugagu

SEQ ID NO: 108 - RNA, 22 nt, Rattus norvegicus
caaagaauucuccuuuugggcu

SEQ ID NO: 109 - RNA, 21 nt, Rattus norvegicus
caucccuugcaugguggaggg

SEQ ID NO: 110 - RNA, 22 nt, Rattus norvegicus
aacuggcccacaaagucccgcu

SEQ ID NO: 111 - RNA, 22 nt, Rattus norvegicus
uguaacagcaacuccaugugga

SEQ ID NO: 112 - RNA, 22 nt, Rattus norvegicus
uagguaguuuccuguuguuggg

SEQ ID NO: 113 - RNA, 23 nt, Rattus norvegicus
ugugcaaaucuaugcaaaacuga

SEQ ID NO: 114 - RNA, 22 nt, Rattus norvegicus
uucccuuugucauccuaugccu

SEQ ID NO: 115 - RNA, 23 nt, Rattus norvegicus
uaaagugcuuauagugcagguag

SEQ ID NO: 116 - RNA, 22 nt, Rattus norvegicus
cugugcgugugacagcggcuga

SEQ ID NO: 117 - RNA, 22 nt, Rattus norvegicus
uagcuuaucagacugauguuga

SEQ ID NO: 118 - RNA, 22 nt, Rattus norvegicus
acagcaggcacagacaggcagu

SEQ ID NO: 119 - RNA, 21 nt, Rattus norvegicus
uugugcuugaucuaaccaugu

SEQ ID NO: 120 - RNA, 21 nt, Rattus norvegicus
aucacauugccagggauuacc

SEQ ID NO: 121 - RNA, 22 nt, Rattus norvegicus
uggcucaguucagcaggaacag

SEQ ID NO: 122 - RNA, 22 nt, Rattus norvegicus
cauugcacuugucucggucuga

SEQ ID NO: 123 - RNA, 22 nt, Rattus norvegicus
uucaaguaauccaggauaggcu

SEQ ID NO: 124 - RNA, 21 nt, Rattus norvegicus
uucaaguaauucaggauaggu

SEQ ID NO: 125 - RNA, 21 nt, Rattus norvegicus
uucacaguggcuaaguucugc

SEQ ID NO: 126 - RNA, 21 nt, Rattus norvegicus
agggcccccccucaauccugu

SEQ ID NO: 127 - RNA, 22 nt, Rattus norvegicus
uagcaccauuugaaaucgguua

SEQ ID NO: 128 - RNA, 23 nt, Rattus norvegicus
cagugcaauaguauugucaaagc

SEQ ID NO: 129 - RNA, 23 nt, Rattus norvegicus
cagugcaaugguauugucaaagc

SEQ ID NO: 130 - RNA, 22 nt, Rattus norvegicus
uguaaacauccucgacuggaag

SEQ ID NO: 131 - RNA, 23 nt, Rattus norvegicus
uguaaacauccuacacucucagc

SEQ ID NO: 132 - RNA, 22 nt, Rattus norvegicus
uguaaacauccccgacuggaag

SEQ ID NO: 133 - RNA, 22 nt, Rattus norvegicus
uguaaacauccuugacuggaag

SEQ ID NO: 134 - RNA, 22 nt, Rattus norvegicus
aaaagcuggguugagagggcga

SEQ ID NO: 135 - RNA, 22 nt, Rattus norvegicus
cagcagcaauucauguuuugga

SEQ ID NO: 136 - RNA, 20 nt, Rattus norvegicus
ccacugccccaggugcugcu

SEQ ID NO: 137 - RNA, 22 nt, Rattus norvegicus
cuggcccucucugcccuuccgu

SEQ ID NO: 138 - RNA, 21 nt, Rattus norvegicus
gccccugggccuauccuagaa

SEQ ID NO: 139 - RNA, 22 nt, Rattus norvegicus
uuuuucauuauugcuccugacc

SEQ ID NO: 140 - RNA, 22 nt, Rattus norvegicus
uggcagugucuuagcugguugu

SEQ ID NO: 141 - RNA, 22 nt, Rattus norvegicus
aaucacuaacuccacugccauc

SEQ ID NO: 142 - RNA, 24 nt, Rattus norvegicus
ucccugaggagcccuuugagccug

SEQ ID NO: 143 - RNA, 22 nt, Rattus norvegicus
aauugcacgguauccaucugua

SEQ ID NO: 144 - RNA, 22 nt, Rattus norvegicus
uaaugccccuaaaaauccuuau

SEQ ID NO: 145 - RNA, 21 nt, Rattus norvegicus
aauauaacacagauggccugu

SEQ ID NO: 146 - RNA, 22 nt, Rattus norvegicus
uuugaaccaucacucgacuccu

SEQ ID NO: 147 - RNA, 22 nt, Rattus norvegicus
cagcagcacacugugguuugua

SEQ ID NO: 148 - RNA, 22 nt, Rattus norvegicus
cacgcucaugcacacacccaca

SEQ ID NO: 149 - RNA, 21 nt, Rattus norvegicus
aauggcgccacuaggguugug

SEQ ID NO: 150 - RNA, 23 nt, Rattus norvegicus
ugacaccugccacccagcccaag

SEQ ID NO: 151 - RNA, 21 nt, Rattus norvegicus
uguucagacugguguccauca

SEQ ID NO: 152 - RNA, 23 nt, Rattus norvegicus
caaagugcuguucgugcagguag

SEQ ID NO: 153 - RNA, 22 nt, Rattus norvegicus
cacccguagaaccgaccuugcg

SEQ ID NO: 154 - RNA, 22 nt, Rattus norvegicus
uagguaguuucguguuguuggg

SEQ ID NO: 155 - RNA, 25 nt, Rattus norvegicus
ucccugaggagcccuuugagccuga

SEQ ID NO: 156 - RNA, 22 nt, Rattus norvegicus
caugccuugaguguaggacugu

SEQ ID NO: 157 - DNA, 57 bp, Rattus norvegicus
ctaaaatagctggaattaccggcagattggtagtggtgagcctatggttttctgaag

SEQ ID NO: 158 - DNA, 72 bp, Rattus norvegicus
acaatgatgacttatgtttttgccgtttacccagctgagggtttctttgaagagagaatcttaagactgagc

SEQ ID NO: 159 - RNA, 22 nt, Rattus norvegicus
cuauacaaucuacugucuuucc

SEQ ID NO: 160 - RNA, 22 nt, Rattus norvegicus
acaagucagguucuugggaccu

SEQ ID NO: 161 - RNA, 22 nt, Rattus norvegicus
acucuuucccuguugcacuacu

SEQ ID NO: 162 - RNA, 22 nt, Rattus norvegicus
uauagggauuggagccguggcg

SEQ ID NO: 163 - RNA, 22 nt, Rattus norvegicus
aucaucgucucaaaugagucuu

SEQ ID NO: 164 - RNA, 22 nt, Rattus norvegicus
cggcuacuucacaacaccaggg

SEQ ID NO: 165 - RNA, 22 nt, Rattus norvegicus
caucuuccagugcaguguugga

SEQ ID NO: 166 - RNA, 23 nt, Rattus norvegicus
ucuggcuccgugucuucacuccc

SEQ ID NO: 167 - RNA, 22 nt, Rattus norvegicus
gcccuaaggugaauuuuuuggg

SEQ ID NO: 168 - RNA, 21 nt, Rattus norvegicus
ugauauguuugauauuggguu

SEQ ID NO: 169 - RNA, 22 nt, Rattus norvegicus
ucggcaacaagaaacugccuga

SEQ ID NO: 170 - RNA, 22 nt, Rattus norvegicus
uggaauguaaggaagugugugg

SEQ ID NO: 171 - RNA, 22 nt, Rattus norvegicus
ccuguucuccauuacuuggcuc

SEQ ID NO: 172 - RNA, 22 nt, Rattus norvegicus
gcugguuucauauggugguuua

SEQ ID NO: 173 - RNA, 21 nt, Rattus norvegicus
aaacaugaagcgcugcaacac

SEQ ID NO: 174 - RNA, 22 nt, Rattus norvegicus
caauguuuccacagugcaucac

SEQ ID NO: 175 - RNA, 22 nt, Rattus norvegicus
aaucacuaaccacacagccagg

SEQ ID NO: 176 - RNA, 21 nt, Rattus norvegicus
acuggacuuggagucagaagg

SEQ ID NO: 177 - RNA, 21 nt, Rattus norvegicus
uauacauacacgcacacauag

SEQ ID NO: 178 - RNA, 22 nt, Rattus norvegicus
auauacauacacacaccaacac

SEQ ID NO: 179 - RNA, 22 nt, Rattus norvegicus
cucacagcucugguccuuggag

SEQ ID NO: 180 - RNA, 22 nt, Rattus norvegicus
cacagcucccaucucagaacaa

SEQ ID NO: 181 - RNA, 22 nt, Rattus norvegicus
gucucggugcaaggacuggagg

SEQ ID NO: 182 - RNA, 22 nt, Rattus norvegicus
aaaggcuaggcucacaaccaaa

SEQ ID NO: 183 - RNA, 18 nt, Rattus norvegicus
gcgugugcuugcuguggg

SEQ ID NO: 184 - RNA, 21 nt, Rattus norvegicus
aacauccugguccuguggaga

SEQ ID NO: 185 - RNA, 19 nt, Rattus norvegicus
ggaggcagaggcaggagga

SEQ ID NO: 186 - RNA, 21 nt, Rattus norvegicus
cuccgugcacacccccgcgug

SEQ ID NO: 187 - RNA, 18 nt, Rattus norvegicus
aucucgcuggggccucca

SEQ ID NO: 188 - RNA, 21 nt, Rattus norvegicus
cagugcaauuaaaagggggaa

SEQ ID NO: 189 - RNA, 20 nt, Rattus norvegicus
cggcucugggucugugggga

SEQ ID NO: 190 - RNA, 24 nt, Rattus norvegicus
gauugcugugcgugcggaaucgac

SEQ ID NO: 191 - RNA, 20 nt, Rattus norvegicus
gaauugaucaggacauaggg

SEQ ID NO: 192 - RNA, 22 nt, Rattus norvegicus
ugaacuauugcaguagccuccu

SEQ ID NO: 193 - RNA, 22 nt, Rattus norvegicus
uauaccucaguuuuaucaggug

SEQ ID NO: 194 - RNA, 22 nt, Rattus norvegicus
uguccucuucucccuccuccca

SEQ ID NO: 195 - RNA, 20 nt, Rattus norvegicus
guagaggagauggcgcaggg

SEQ ID NO: 196 - RNA, 22 nt, Rattus norvegicus
gcaugacaccacacuggguaga

SEQ ID NO: 197 - RNA, 22 nt, Rattus norvegicus
acugcugagcuagcacuucccg

SEQ ID NO: 198 - RNA, 22 nt, Rattus norvegicus
caagcucgugucuguggguccg

SEQ ID NO: 199 - RNA, 22 nt, Rattus norvegicus
ugauagacgccaauuuggguag

SEQ ID NO: 200 - RNA, 22 nt, Rattus norvegicus
uauucauuuacuccccagccua

SEQ ID NO: 201 - RNA, 22 nt, Rattus norvegicus
gaaagacgccaaacuggguaga

SEQ ID NO: 202 - DNA, 57 bp, Rattus norvegicus; identical to SEQ ID NO: 157

SEQ ID NO: 203 - DNA, 72 bp, Rattus norvegicus; identical to SEQ ID NO: 158

SEQ ID NO: 204 - RNA, 22 nt, Rattus norvegicus
uacaguacugugauaacugaag

SEQ ID NO: 205 - RNA, 23 nt, Rattus norvegicus
uacccuguagauccgaauuugug

SEQ ID NO: 206 - RNA, 23 nt, Rattus norvegicus
uacccuguagaaccgaauuugug

SEQ ID NO: 207 - RNA, 22 nt, Rattus norvegicus
acaggugagguucuugggagcc

SEQ ID NO: 208 - RNA, 24 nt, Rattus norvegicus
ucccugagacccuuuaaccuguga

SEQ ID NO: 209 - RNA, 22 nt, Rattus norvegicus
acggguuaggcucuugggagcu

SEQ ID NO: 210 - RNA, 21 nt, Rattus norvegicus
ucacagucaaccggucucuuu

SEQ ID NO: 211 - RNA, 22 nt, Rattus norvegicus
aagcccuuaccccaaaaagcau

SEQ ID NO: 212 - RNA, 23 nt, Rattus norvegicus
agcugguguugugaaucaggccg

SEQ ID NO: 213 - RNA, 23 nt, Rattus norvegicus
uguaguguuuccuacuuuaugga

SEQ ID NO: 214 - RNA, 21 nt, Rattus norvegicus
cauaaaguagaaagcacuacu

SEQ ID NO: 215 - RNA, 21 nt, Rattus norvegicus
ugagaugaagcacuguagcuc

SEQ ID NO: 216 - RNA, 22 nt, Rattus norvegicus
ugagaacugaauuccauggguu

SEQ ID NO: 217 - RNA, 22 nt, Rattus norvegicus
gugugcggaaaugcuucugcua

SEQ ID NO: 218 - RNA, 22 nt, Rattus norvegicus
ucagugcacuacagaacuuugu

SEQ ID NO: 219 - RNA, 22 nt, Rattus norvegicus
ucagugcaucacagaacuuugu

SEQ ID NO: 220 - RNA, 21 nt, Rattus norvegicus
cuagacugaggcuccuugagg

SEQ ID NO: 221 - RNA, 25 nt, Rattus norvegicus
uuuggcaaugguagaacucacaccg

SEQ ID NO: 222 - RNA, 22 nt, Rattus norvegicus
ucgugucuuguguugcagccgg

SEQ ID NO: 223 - RNA, 21 nt, Rattus norvegicus
caucccuugcaugguggaggg

SEQ ID NO: 224 - RNA, 23 nt, Rattus norvegicus
uaaggugcaucuagugcagauag

SEQ ID NO: 225 - RNA, 22 nt, Rattus norvegicus
ugauauguuugauauauuaggu

SEQ ID NO: 226 - RNA, 22 nt, Rattus norvegicus
uagguaguuuccuguuguuggg

SEQ ID NO: 227 - RNA, 22 nt, Rattus norvegicus
uucaccaccuucuccacccagc

SEQ ID NO: 228 - RNA, 22 nt, Rattus norvegicus
acaguagucugcacauugguua

SEQ ID NO: 229 - RNA, 23 nt, Rattus norvegicus
uaauacugccggguaaugaugga

SEQ ID NO: 230 - RNA, 22 nt, Rattus norvegicus
uucccuuugucauccuaugccu

SEQ ID NO: 231 - RNA, 22 nt, Rattus norvegicus
cugugcgugugacagcggcuga

SEQ ID NO: 232 - RNA, 22 nt, Rattus norvegicus
uagcuuaucagacugauguuga

SEQ ID NO: 233 - RNA, 21 nt, Rattus norvegicus
agcuacaucuggcuacugggu

SEQ ID NO: 234 - RNA, 21 nt, Rattus norvegicus
aucacauugccagggauuucc

SEQ ID NO: 235 - RNA, 21 nt, Rattus norvegicus
aucacauugccagggauuacc

SEQ ID NO: 236 - RNA, 22 nt, Rattus norvegicus
uucaaguaauccaggauaggcu

SEQ ID NO: 237 - RNA, 23 nt, Rattus norvegicus
uagcaccauuugaaaucaguguu

SEQ ID NO: 238 - RNA, 22 nt, Rattus norvegicus
uagcaccauuugaaaucgguua

SEQ ID NO: 239 - RNA, 22 nt, Rattus norvegicus
aaaagcuggguugagagggcga

SEQ ID NO: 240 - RNA, 22 nt, Rattus norvegicus
cagcagcaauucauguuuugga

SEQ ID NO: 241 - RNA, 23 nt, Rattus norvegicus
cgcauccccuagggcauuggugu

SEQ ID NO: 242 - RNA, 22 nt, Rattus norvegicus
cuagguauggucccagggaucc

SEQ ID NO: 243 - RNA, 22 nt, Rattus norvegicus
uuuuucauuauugcuccugacc

SEQ ID NO: 244 - RNA, 23 nt, Rattus norvegicus
ucccuguccuccaggagcucacg

SEQ ID NO: 245 - RNA, 22 nt, Rattus norvegicus
gcugaccccuaguccagugcuu

SEQ ID NO: 246 - RNA, 22 nt, Rattus norvegicus
uucacaaagcccauacacuuuc

SEQ ID NO: 247 - RNA, 24 nt, Rattus norvegicus
ucccugaggagcccuuugagccug

SEQ ID NO: 248 - RNA, 22 nt, Rattus norvegicus
uuaucagaaucuccagggguac

SEQ ID NO: 249 - RNA, 22 nt, Rattus norvegicus
aacacaccuguucaaggauuca

SEQ ID NO: 250 - RNA, 23 nt, Rattus norvegicus
uguaaacaauuccuaggcaaugu

SEQ ID NO: 251 - RNA, 22 nt, Rattus norvegicus
uaauacugucugguaaugccgu

SEQ ID NO: 252 - RNA, 22 nt, Rattus norvegicus
uuuugcgauguguuccuaauau

SEQ ID NO: 253 - RNA, 22 nt, Rattus norvegicus
ugaaacauacacgggaaaccuc

SEQ ID NO: 254 - RNA, 22 nt, Rattus norvegicus
aaugcaccugggcaaggguuca

SEQ ID NO: 255 - RNA, 23 nt, Rattus norvegicus
uagcagcgggaacaguacugcag

SEQ ID NO: 256 - RNA, 22 nt, Rattus norvegicus
ugugacagauugauaacugaaa

SEQ ID NO: 257 - RNA, 22 nt, Rattus norvegicus
ccuguugaacaacugaacccaa

SEQ ID NO: 258 - RNA, 22 nt, Rattus norvegicus
uacaguuguucaaccaguuacu

SEQ ID NO: 259 - RNA, 22 nt, Rattus norvegicus
uacgucaucgucgucaucguua

SEQ ID NO: 260 - RNA, 21 nt, Rattus norvegicus
aauggcgccacuaggguugug

SEQ ID NO: 261 - RNA, 23 nt, Rattus norvegicus
ugacaccugccacccagcccaag

SEQ ID NO: 262 - RNA, 21 nt, Rattus norvegicus
ucaauggcugaggugaggcac

SEQ ID NO: 263 - RNA, 21 nt, Rattus norvegicus
uguucagacugguguccauca

SEQ ID NO: 264 - RNA, 22 nt, Rattus norvegicus
ugcggggcuagggcuaacagca

SEQ ID NO: 265 - RNA, 22 nt, Rattus norvegicus
uaacugcaacagcucucaguau

SEQ ID NO: 266 - RNA, 22 nt, Rattus norvegicus
uaacugcaacaucucucaguau

SEQ ID NO: 267 - RNA, 22 nt, Rattus norvegicus
ugagguaguaaguuguauuguu

SEQ ID NO: 268 - RNA, 21 nt, Rattus norvegicus
ugauauguuugauauuagguu

SEQ ID NO: 269 - RNA, 23 nt, Rattus norvegicus
gcuucuccuggcucuccucccuu

SEQ ID NO: 270 - RNA, 20 nt, Rattus norvegicus
guggugugcuaguuacuuuu

SEQ ID NO: 271 - RNA, 23 nt, Rattus norvegicus
ugagcgccucgacgacagagcca

SEQ ID NO: 272 - RNA, 22 nt, Rattus norvegicus
cccugaacuaggggucuggaga

SEQ ID NO: 273 - RNA, 25 nt, Rattus norvegicus
ucccugaggagcccuuugagccuga

SEQ ID NO: 274 - RNA, 21 nt, Rattus norvegicus
ugugaugugugcauguacaug

SEQ ID NO: 275 - RNA, 22 nt, Rattus norvegicus
gaaagacaccauacugaauaga

SEQ ID NO: 276 - DNA, 48 bp, Rattus norvegicus
gctgtactgacttgatgaaagtacttttgaacccttttccatctgatg

SEQ ID NO: 277 - RNA, 22 nt, Rattus norvegicus
cuauacaaucuauugccuuccc

SEQ ID NO: 278 - RNA, 22 nt, Rattus norvegicus
acuguacaggccacugccuugc

SEQ ID NO: 279 - RNA, 22 nt, Rattus norvegicus
cugcgcaagcuacugccuugcu

SEQ ID NO: 280 - RNA, 22 nt, Rattus norvegicus
ccgcacuguggguacuugcugc

SEQ ID NO: 281 - RNA, 22 nt, Rattus norvegicus
caaauucguaucuaggggaaua

SEQ ID NO: 282 - RNA, 22 nt, Rattus norvegicus
cagauucgauucuaggggaaua

SEQ ID NO: 283 - RNA, 22 nt, Rattus norvegicus
acucuuucccuguugcacuacu

SEQ ID NO: 284 - RNA, 22 nt, Rattus norvegicus
uauagggauuggagccguggcg

SEQ ID NO: 285 - RNA, 23 nt, Rattus norvegicus
ucuggcuccgugucuucacuccc

SEQ ID NO: 286 - RNA, 22 nt, Rattus norvegicus
cgaaucauuauuugcugcucua

SEQ ID NO: 287 - RNA, 22 nt, Rattus norvegicus
ccaguauugacugugcugcuga

SEQ ID NO: 288 - RNA, 22 nt, Rattus norvegicus
acugcagugagggcacuuguag

SEQ ID NO: 289 - RNA, 22 nt, Rattus norvegicus
acugcccuaagugcuccuucug

SEQ ID NO: 290 - RNA, 22 nt, Rattus norvegicus
gcugcacuuggauuucguuccc

SEQ ID NO: 291 - RNA, 23 nt, Rattus norvegicus
cccaguguuuagacuaccuguuc

SEQ ID NO: 292 - RNA, 22 nt, Rattus norvegicus
uggaauguaaggaagugugugg

SEQ ID NO: 293 - RNA, 22 nt, Rattus norvegicus
ugccugucuacacuugcugugc

SEQ ID NO: 294 - RNA, 22 nt, Rattus norvegicus
aaacaugguuccgucaagcacc

SEQ ID NO: 295 - RNA, 23 nt, Rattus norvegicus
gugccuacugagcugauaucagu

SEQ ID NO: 296 - RNA, 22 nt, Rattus norvegicus
ccuguucuccauuacuuggcuc

SEQ ID NO: 297 - RNA, 22 nt, Rattus norvegicus
caucaaaguggaggcccucucu

SEQ ID NO: 298 - RNA, 22 nt, Rattus norvegicus
uauacauacacacauacccaua

SEQ ID NO: 299 - RNA, 22 nt, Rattus norvegicus
acugauuucuuuugguguucag

SEQ ID NO: 300 - RNA, 22 nt, Rattus norvegicus
gcugguuucauauggugguuua

SEQ ID NO: 301 - RNA, 22 nt, Rattus norvegicus
ugaccgauuucuccugguguuc

SEQ ID NO: 302 - RNA, 22 nt, Rattus norvegicus
cuuucagucggauguuugcagc

SEQ ID NO: 303 - RNA, 22 nt, Rattus norvegicus
cugggauguggauguuuacguc

SEQ ID NO: 304 - RNA, 22 nt, Rattus norvegicus
cugggagaggguuguuuacucc

SEQ ID NO: 305 - RNA, 22 nt, Rattus norvegicus
cuuucagucggauguuuacagc

SEQ ID NO: 306 - RNA, 21 nt, Rattus norvegicus
aaacaugaagcgcugcaacac

SEQ ID NO: 307 - RNA, 21 nt, Rattus norvegicus
ccucugggcccuuccuccagu

SEQ ID NO: 308 - RNA, 23 nt, Rattus norvegicus
gcaaagcacagggccugcagaga

SEQ ID NO: 309 - RNA, 22 nt, Rattus norvegicus
auauaauacaaccugcuaagug

SEQ ID NO: 310 - RNA, 21 nt, Rattus norvegicus
acuggacuuggagucagaagg

SEQ ID NO: 311 - RNA, 21 nt, Rattus norvegicus
aucgggaaugucguguccgcc

SEQ ID NO: 312 - RNA, 21 nt, Rattus norvegicus
uauacauacacgcacacauag

SEQ ID NO: 313 - RNA, 22 nt, Rattus norvegicus
auauacauacacacaccuacac

SEQ ID NO: 314 - RNA, 22 nt, Rattus norvegicus
auauacauacacacaccaacac

SEQ ID NO: 315 - RNA, 22 nt, Rattus norvegicus
gaguauuguuuccacugccugg

SEQ ID NO: 316 - RNA, 22 nt, Rattus norvegicus
cucacagcucugguccuuggag

SEQ ID NO: 317 - RNA, 22 nt, Rattus norvegicus
cacagcucccaucucagaacaa

SEQ ID NO: 318 - RNA, 22 nt, Rattus norvegicus
gucucggugcaaggacuggagg

SEQ ID NO: 319 - RNA, 21 nt, Rattus norvegicus
aucucuuugagcgccucacuc

SEQ ID NO: 320 - RNA, 20 nt, Rattus norvegicus
aggcagugcgaccuggcucg

SEQ ID NO: 321 - RNA, 18 nt, Rattus norvegicus
aucucgcuggggccucca

SEQ ID NO: 322 - RNA, 21 nt, Rattus norvegicus
cagugcaauuaaaagggggaa

SEQ ID NO: 323 - RNA, 22 nt, Rattus norvegicus
cuguugccacuaaccucaaccu

SEQ ID NO: 324 - RNA, 20 nt, Rattus norvegicus
cggcucugggucugugggga

SEQ ID NO: 325 - RNA, 24 nt, Rattus norvegicus
gauugcugugcgugcggaaucgac

SEQ ID NO: 326 - RNA, 22 nt, Rattus norvegicus
uauaccucaguuuuaucaggug

SEQ ID NO: 327 - RNA, 20 nt, Rattus norvegicus
guagaggagauggcgcaggg

SEQ ID NO: 328 - RNA, 22 nt, Rattus norvegicus
auaaagcuagauaaccgaaagu

SEQ ID NO: 329 - RNA, 22 nt, Rattus norvegicus
caagcucgugucuguggguccg

SEQ ID NO: 330 - RNA, 22 nt, Rattus norvegicus
cacuagauugugagcuccugga

SEQ ID NO: 331 - RNA, 22 nt, Rattus norvegicus
ugauagacgccaauuuggguag

SEQ ID NO: 332 - RNA, 22 nt, Rattus norvegicus
caagcucguuucuaugggucug

SEQ ID NO: 333 - DNA, 57 bp, Rattus norvegicus; identical to SEQ ID NO: 157

SEQ ID NO: 334 - RNA, 22 nt, Rattus norvegicus
ugagaacugaauuccauggguu

SEQ ID NO: 335 - RNA, 21 nt, Rattus norvegicus
caucccuugcaugguggaggg

SEQ ID NO: 336 - RNA, 22 nt, Rattus norvegicus
uagguaguuuccuguuguuggg

SEQ ID NO: 337 - RNA, 22 nt, Rattus norvegicus
uucccuuugucauccuaugccu

SEQ ID NO: 338 - RNA, 22 nt, Rattus norvegicus
cugugcgugugacagcggcuga

SEQ ID NO: 339 - RNA, 21 nt, Rattus norvegicus
aucacauugccagggauuacc

SEQ ID NO: 340 - RNA, 22 nt, Rattus norvegicus
uagcaccauuugaaaucgguua

SEQ ID NO: 341 - RNA, 22 nt, Rattus norvegicus
aaaagcuggguugagagggcga

SEQ ID NO: 342 - RNA, 22 nt, Rattus norvegicus
uuuuucauuauugcuccugacc

SEQ ID NO: 343 - RNA, 21 nt, Rattus norvegicus
aauggcgccacuaggguugug

SEQ ID NO: 344 - RNA, 22 nt, Rattus norvegicus
uauagggauuggagccguggcg

SEQ ID NO: 345 - RNA, 22 nt, Rattus norvegicus
uggaauguaaggaagugugugg

SEQ ID NO: 346 - RNA, 22 nt, Rattus norvegicus
ccuguucuccauuacuuggcuc

SEQ ID NO: 347 - RNA, 22 nt, Rattus norvegicus
gcugguuucauauggugguuua

SEQ ID NO: 348 - RNA, 21 nt, Rattus norvegicus
acuggacuuggagucagaagg

SEQ ID NO: 349 - RNA, 21 nt, Rattus norvegicus
uauacauacacgcacacauag

SEQ ID NO: 350 - RNA, 22 nt, Rattus norvegicus
auauacauacacacaccaacac

SEQ ID NO: 351 - RNA, 22 nt, Rattus norvegicus
cucacagcucugguccuuggag

SEQ ID NO: 352 - RNA, 22 nt, Rattus norvegicus
cacagcucccaucucagaacaa

SEQ ID NO: 353 - RNA, 18 nt, Rattus norvegicus
aucucgcuggggccucca

SEQ ID NO: 354 - RNA, 21 nt, Rattus norvegicus
cagugcaauuaaaagggggaa

SEQ ID NO: 355 - RNA, 20 nt, Rattus norvegicus
cggcucugggucugugggga

SEQ ID NO: 356 - RNA, 24 nt, Rattus norvegicus
gauugcugugcgugcggaaucgac

SEQ ID NO: 357 - RNA, 20 nt, Rattus norvegicus
guagaggagauggcgcaggg

SEQ ID NO: 358 - RNA, 22 nt, Rattus norvegicus
caagcucgugucuguggguccg

SEQ ID NO: 359 - DNA, 57 bp, Rattus norvegicus; identical to SEQ ID NO: 157

SEQ ID NO: 360 - RNA, 23 nt, Rattus norvegicus
caaagugcuaacagugcagguag

SEQ ID NO: 361 - RNA, 21 nt, Rattus norvegicus
uaaagugcugacagugcagau

SEQ ID NO: 362 - RNA, 23 nt, Rattus norvegicus
uacccuguagauccgaauuugug

SEQ ID NO: 363 - RNA, 22 nt, Rattus norvegicus
ucguaccgugaguaauaaugcg

SEQ ID NO: 364 - RNA, 22 nt, Rattus norvegicus
cagugcaauguuaaaagggcau

SEQ ID NO: 365 - RNA, 22 nt, Rattus norvegicus
cagugguuuuacccuaugguag

SEQ ID NO: 366 - RNA, 23 nt, Rattus norvegicus
guccaguuuucccaggaaucccu

SEQ ID NO: 367 - RNA, 22 nt, Rattus norvegicus
ugagaacugaauuccauggguu

SEQ ID NO: 368 - RNA, 22 nt, Rattus norvegicus
ugagaacugaauuccauaggcu

SEQ ID NO: 369 - RNA, 23 nt, Rattus norvegicus
uuaaugcuaauugugauaggggu

SEQ ID NO: 370 - RNA, 22 nt, Rattus norvegicus
uagcagcacaucaugguuuaca

SEQ ID NO: 371 - RNA, 22 nt, Rattus norvegicus
uagcagcacguaaauauuggcg

SEQ ID NO: 372 - RNA, 23 nt, Rattus norvegicus
aacauucaacgcugucggugagu

SEQ ID NO: 373 - RNA, 21 nt, Rattus norvegicus
caucccuugcaugguggaggg

SEQ ID NO: 374 - RNA, 22 nt, Rattus norvegicus
uagguaguuuccuguuguuggg

SEQ ID NO: 375 - RNA, 23 nt, Rattus norvegicus
ugugcaaaucuaugcaaaacuga

SEQ ID NO: 376 - RNA, 22 nt, Rattus norvegicus
uucccuuugucauccuaugccu

SEQ ID NO: 377 - RNA, 23 nt, Rattus norvegicus
uaaagugcuuauagugcagguag

SEQ ID NO: 378 - RNA, 22 nt, Rattus norvegicus
cugugcgugugacagcggcuga

SEQ ID NO: 379 - RNA, 22 nt, Rattus norvegicus
uagcuuaucagacugauguuga

SEQ ID NO: 380 - RNA, 21 nt, Rattus norvegicus
uugugcuugaucuaaccaugu

SEQ ID NO: 381 - RNA, 21 nt, Rattus norvegicus
aucacauugccagggauuacc

SEQ ID NO: 382 - RNA, 22 nt, Rattus norvegicus
uggcucaguucagcaggaacag

SEQ ID NO: 383 - RNA, 22 nt, Rattus norvegicus
cauugcacuugucucggucuga

SEQ ID NO: 384 - RNA, 21 nt, Rattus norvegicus
uucacaguggcuaaguucugc

SEQ ID NO: 385 - RNA, 22 nt, Rattus norvegicus
uagcaccauuugaaaucgguua

SEQ ID NO: 386 - RNA, 23 nt, Rattus norvegicus
uguaaacauccuacacucucagc

SEQ ID NO: 387 - RNA, 22 nt, Rattus norvegicus
uguaaacauccccgacuggaag

SEQ ID NO: 388 - RNA, 22 nt, Rattus norvegicus
uguaaacauccuugacuggaag

SEQ ID NO: 389 - RNA, 22 nt, Rattus norvegicus
cuccugacuccagguccugugu

SEQ ID NO: 390 - RNA, 22 nt, Rattus norvegicus
aaaagcuggguugagagggcga

SEQ ID NO: 391 - RNA, 20 nt, Rattus norvegicus
ccacugccccaggugcugcu

SEQ ID NO: 392 - RNA, 22 nt, Rattus norvegicus
cuggcccucucugcccuuccgu

SEQ ID NO: 393 - RNA, 21 nt, Rattus norvegicus
gccccugggccuauccuagaa

SEQ ID NO: 394 - RNA, 22 nt, Rattus norvegicus
uuuuucauuauugcuccugacc

SEQ ID NO: 395 - RNA, 21 nt, Rattus norvegicus
aauauaacacagauggccugu

SEQ ID NO: 396 - RNA, 22 nt, Rattus norvegicus
uuugaaccaucacucgacuccu

SEQ ID NO: 397 - RNA, 22 nt, Rattus norvegicus
cacgcucaugcacacacccaca

SEQ ID NO: 398 - RNA, 21 nt, Rattus norvegicus
aauggcgccacuaggguugug

SEQ ID NO: 399 - RNA, 23 nt, Rattus norvegicus
caaagugcuguucgugcagguag

SEQ ID NO: 400 - RNA, 22 nt, Rattus norvegicus
cacccguagaaccgaccuugcg

SEQ ID NO: 401 - RNA, 22 nt, Rattus norvegicus
uagguaguuucguguuguuggg

SEQ ID NO: 402 - RNA, 22 nt, Rattus norvegicus
cuauacaaucuacugucuuucc

SEQ ID NO: 403 - RNA, 22 nt, Rattus norvegicus
acaagucagguucuugggaccu

SEQ ID NO: 404 - RNA, 22 nt, Rattus norvegicus
uauagggauuggagccguggcg

SEQ ID NO: 405 - RNA, 22 nt, Rattus norvegicus
aucaucgucucaaaugagucuu

SEQ ID NO: 406 - RNA, 22 nt, Rattus norvegicus
cggcuacuucacaacaccaggg

SEQ ID NO: 407 - RNA, 22 nt, Rattus norvegicus
caucuuccagugcaguguugga

SEQ ID NO: 408 - RNA, 22 nt, Rattus norvegicus
gcccuaaggugaauuuuuuggg

SEQ ID NO: 409 - RNA, 21 nt, Rattus norvegicus
ugauauguuugauauuggguu

SEQ ID NO: 410 - RNA, 22 nt, Rattus norvegicus
uggaauguaaggaagugugugg

SEQ ID NO: 411 - RNA, 22 nt, Rattus norvegicus
ccuguucuccauuacuuggcuc

SEQ ID NO: 412 - RNA, 22 nt, Rattus norvegicus
gcugguuucauauggugguuua

SEQ ID NO: 413 - RNA, 22 nt, Rattus norvegicus
aaucacuaaccacacagccagg

SEQ ID NO: 414 - RNA, 21 nt, Rattus norvegicus
acuggacuuggagucagaagg

SEQ ID NO: 415 - RNA, 21 nt, Rattus norvegicus
uauacauacacgcacacauag

SEQ ID NO: 416 - RNA, 22 nt, Rattus norvegicus
auauacauacacacaccaacac

SEQ ID NO: 417 - RNA, 22 nt, Rattus norvegicus
cucacagcucugguccuuggag

SEQ ID NO: 418 - RNA, 22 nt, Rattus norvegicus
cacagcucccaucucagaacaa

SEQ ID NO: 419 - RNA, 22 nt, Rattus norvegicus
aaaggcuaggcucacaaccaaa

SEQ ID NO: 420 - RNA, 18 nt, Rattus norvegicus
gcgugugcuugcuguggg

SEQ ID NO: 421 - RNA, 21 nt, Rattus norvegicus
aacauccugguccuguggaga

SEQ ID NO: 422 - RNA, 21 nt, Rattus norvegicus
cuccgugcacacccccgcgug

SEQ ID NO: 423 - RNA, 18 nt, Rattus norvegicus
aucucgcuggggccucca

SEQ ID NO: 424 - RNA, 21 nt, Rattus norvegicus
cagugcaauuaaaagggggaa

SEQ ID NO: 425 - RNA, 20 nt, Rattus norvegicus
cggcucugggucugugggga

SEQ ID NO: 426 - RNA, 24 nt, Rattus norvegicus
gauugcugugcgugcggaaucgac

SEQ ID NO: 427 - RNA, 20 nt, Rattus norvegicus
gaauugaucaggacauaggg

SEQ ID NO: 428 - RNA, 22 nt, Rattus norvegicus
ugaacuauugcaguagccuccu

SEQ ID NO: 429 - RNA, 22 nt, Rattus norvegicus
uguccucuucucccuccuccca

SEQ ID NO: 430 - RNA, 20 nt, Rattus norvegicus
guagaggagauggcgcaggg

SEQ ID NO: 431 - RNA, 22 nt, Rattus norvegicus
gcaugacaccacacuggguaga

SEQ ID NO: 432 - RNA, 22 nt, Rattus norvegicus
acugcugagcuagcacuucccg

SEQ ID NO: 433 - RNA, 22 nt, Rattus norvegicus
caagcucgugucuguggguccg

SEQ ID NO: 434 - RNA, 23 nt, Rattus norvegicus
uacccuguagauccgaauuugug

SEQ ID NO: 435 - RNA, 24 nt, Rattus norvegicus
ucccugagacccuuuaaccuguga

SEQ ID NO: 436 - RNA, 21 nt, Rattus norvegicus
ucacagucaaccggucucuuu

SEQ ID NO: 437 - RNA, 22 nt, Rattus norvegicus
aagcccuuaccccaaaaagcau

SEQ ID NO: 438 - RNA, 22 nt, Rattus norvegicus
ugagaacugaauuccauggguu

SEQ ID NO: 439 - RNA, 21 nt, Rattus norvegicus
cuagacugaggcuccuugagg

SEQ ID NO: 440 - RNA, 22 nt, Rattus norvegicus
ucgugucuuguguugcagccgg

SEQ ID NO: 441 - RNA, 21 nt, Rattus norvegicus
caucccuugcaugguggaggg

SEQ ID NO: 442 - RNA, 22 nt, Rattus norvegicus
uucaccaccuucuccacccagc

SEQ ID NO: 443 - RNA, 22 nt, Rattus norvegicus
acaguagucugcacauugguua

SEQ ID NO: 444 - RNA, 22 nt, Rattus norvegicus
uauucauuuacuccccagccua

SEQ ID NO: 445 - RNA, 22 nt, Rattus norvegicus
gaaagacgccaaacuggguaga

SEQ ID NO: 446 - DNA, 57 bp, Rattus norvegicus; identical to SEQ ID NO: 157

SEQ ID NO: 447 - DNA, 72 bp, Rattus norvegicus; identical to SEQ ID NO: 158

SEQ ID NO: 448 - RNA, 23 nt, Rattus norvegicus
uacccuguagauccgaauuugug

SEQ ID NO: 449 - RNA, 23 nt, Rattus norvegicus
uguaguguuuccuacuuuaugga

SEQ ID NO: 450 - RNA, 22 nt, Rattus norvegicus
ugagaacugaauuccauggguu

SEQ ID NO: 451 - RNA, 22 nt, Rattus norvegicus
ucagugcaucacagaacuuugu

SEQ ID NO: 452 - RNA, 21 nt, Rattus norvegicus
caucccuugcaugguggaggg

SEQ ID NO: 453 - RNA, 22 nt, Rattus norvegicus
uagguaguuuccuguuguuggg

SEQ ID NO: 454 - RNA, 22 nt, Rattus norvegicus
uucccuuugucauccuaugccu

SEQ ID NO: 455 - RNA, 22 nt, Rattus norvegicus
cugugcgugugacagcggcuga

SEQ ID NO: 456 - RNA, 22 nt, Rattus norvegicus
uagcuuaucagacugauguuga

SEQ ID NO: 457 - RNA, 21 nt, Rattus norvegicus
aucacauugccagggauuacc

SEQ ID NO: 458 - RNA, 22 nt, Rattus norvegicus
uucaaguaauccaggauaggcu

SEQ ID NO: 459 - RNA, 22 nt, Rattus norvegicus
uagcaccauuugaaaucgguua

SEQ ID NO: 460 - RNA, 22 nt, Rattus norvegicus
aaaagcuggguugagagggcga

SEQ ID NO: 461 - RNA, 22 nt, Rattus norvegicus
cagcagcaauucauguuuugga

SEQ ID NO: 462 - RNA, 22 nt, Rattus norvegicus
uuuuucauuauugcuccugacc

SEQ ID NO: 463 - RNA, 24 nt, Rattus norvegicus
ucccugaggagcccuuugagccug

SEQ ID NO: 464 - RNA, 21 nt, Rattus norvegicus
aauggcgccacuaggguugug

SEQ ID NO: 465 - RNA, 23 nt, Rattus norvegicus
ugacaccugccacccagcccaag

SEQ ID NO: 466 - RNA, 21 nt, Rattus norvegicus
uguucagacugguguccauca

SEQ ID NO: 467 - RNA, 24 nt, Rattus norvegicus
ucccugaggagcccuuugagccug

SEQ ID NO: 468 - RNA, 22 nt, Rattus norvegicus
acucuuucccuguugcacuacu

SEQ ID NO: 469 - RNA, 22 nt, Rattus norvegicus
uauagggauuggagccguggcg

SEQ ID NO: 470 - RNA, 23 nt, Rattus norvegicus
ucuggcuccgugucuucacuccc

SEQ ID NO: 471 - RNA, 22 nt, Rattus norvegicus
uggaauguaaggaagugugugg

SEQ ID NO: 472 - RNA, 22 nt, Rattus norvegicus
ccuguucuccauuacuuggcuc

SEQ ID NO: 473 - RNA, 22 nt, Rattus norvegicus
gcugguuucauauggugguuua

SEQ ID NO: 474 - RNA, 21 nt, Rattus norvegicus
aaacaugaagcgcugcaacac

SEQ ID NO: 475 - RNA, 21 nt, Rattus norvegicus
acuggacuuggagucagaagg

SEQ ID NO: 476 - RNA, 21 nt, Rattus norvegicus
uauacauacacgcacacauag

SEQ ID NO: 477 - RNA, 22 nt, Rattus norvegicus
auauacauacacacaccaacac

SEQ ID NO: 478 - RNA, 22 nt, Rattus norvegicus
cucacagcucugguccuuggag

SEQ ID NO: 479 - RNA, 22 nt, Rattus norvegicus
cacagcucccaucucagaacaa

SEQ ID NO: 480 - RNA, 22 nt, Rattus norvegicus
gucucggugcaaggacuggagg

SEQ ID NO: 481 - RNA, 18 nt, Rattus norvegicus
aucucgcuggggccucca

SEQ ID NO: 482 - RNA, 21 nt, Rattus norvegicus
cagugcaauuaaaagggggaa

SEQ ID NO: 483 - RNA, 20 nt, Rattus norvegicus
cggcucugggucugugggga

SEQ ID NO: 484 - RNA, 24 nt, Rattus norvegicus
gauugcugugcgugcggaaucgac

SEQ ID NO: 485 - RNA, 22 nt, Rattus norvegicus
uauaccucaguuuuaucaggug

SEQ ID NO: 486 - RNA, 20 nt, Rattus norvegicus
guagaggagauggcgcaggg

SEQ ID NO: 487 - RNA, 22 nt, Rattus norvegicus
caagcucgugucuguggguccg

SEQ ID NO: 488 - RNA, 22 nt, Rattus norvegicus
ugauagacgccaauuuggguag

SEQ ID NO: 489 - DNA, 57 bp, Rattus norvegicus; identical to SEQ ID NO: 157

SEQ ID NO: 490 - RNA, 22 nt, Rattus norvegicus
caagcucgugucuguggguccg

SEQ ID NO: 491 - DNA, 57 bp, Rattus norvegicus; identical to SEQ ID NO: 157

SEQ ID NO: 492 - RNA, 23 nt, Homo sapiens
uuaaugcuaaucgugauaggggu

SEQ ID NO: 493 - RNA, 22 nt, Homo sapiens
ugagguaguagguugugugguu

SEQ ID NO: 494 - RNA, 22 nt, Homo sapiens
agagguaguagguugcauaguu

SEQ ID NO: 495 - RNA, 22 nt, Homo sapiens
ugagguaggagguuguauaguu

SEQ ID NO: 496 - RNA, 22 nt, Homo sapiens
aacccguagauccgaacuugug

SEQ ID NO: 497 - RNA, 24 nt, Homo sapiens
ucccugagacccuuuaaccuguga

SEQ ID NO: 498 - RNA, 22 nt, Homo sapiens
ucccugagacccuaacuuguga

SEQ ID NO: 499 - RNA, 22 nt, Homo sapiens
ucguaccgugaguaauaaugcg

SEQ ID NO: 500 - RNA, 22 nt, Homo sapiens
ugugacugguugaccagagggg

SEQ ID NO: 501 - RNA, 23 nt, Homo sapiens
agcugguguugugaaucaggccg

SEQ ID NO: 502 - RNA, 22 nt, Homo sapiens
ucuacagugcacgugucuccag

SEQ ID NO: 503 - RNA, 22 nt, Homo sapiens
cagugguuuuacccuaugguag

SEQ ID NO: 504 - RNA, 21 nt, Homo sapiens
ugagaugaagcacuguagcuc

SEQ ID NO: 505 - RNA, 23 nt, Homo sapiens
guccaguuuucccaggaaucccu

SEQ ID NO: 506 - RNA, 23 nt, Homo sapiens
ucuggcuccgugucuucacuccc

SEQ ID NO: 507 - RNA, 21 nt, Homo sapiens
ucagugcaugacagaacuugg

SEQ ID NO: 508 - RNA, 22 nt, Homo sapiens
uugcauagucacaaaagugauc

SEQ ID NO: 509 - RNA, 22 nt, Homo sapiens
uagcagcacaucaugguuuaca

SEQ ID NO: 510 - RNA, 22 nt, Homo sapiens
uagcagcacguaaauauuggcg

SEQ ID NO: 511 - RNA, 23 nt, Homo sapiens
caaagugcuuacagugcagguag

SEQ ID NO: 512 - RNA, 23 nt, Homo sapiens
aacauucaacgcugucggugagu

SEQ ID NO: 513 - RNA, 22 nt, Homo sapiens
uggacggagaacugauaagggu

SEQ ID NO: 514 - RNA, 22 nt, Homo sapiens
caaagaauucuccuuuugggcu

SEQ ID NO: 515 - RNA, 23 nt, Homo sapiens
caacggaaucccaaaagcagcug

SEQ ID NO: 516 - RNA, 22 nt, Homo sapiens
aacuggcccucaaagucccgcu

SEQ ID NO: 517 - RNA, 22 nt, Homo sapiens
uguaacagcaacuccaugugga

SEQ ID NO: 518 - RNA, 22 nt, Homo sapiens
uucaccaccuucuccacccagc

SEQ ID NO: 519 - RNA, 22 nt, Homo sapiens
acaguagucugcacauugguua

SEQ ID NO: 520 - RNA, 23 nt, Homo sapiens
ugugcaaauccaugcaaaacuga

SEQ ID NO: 521 - RNA, 22 nt, Homo sapiens
uucccuuugucauccuaugccu

SEQ ID NO: 522 - RNA, 22 nt, Homo sapiens
auaagacgagcaaaaagcuugu

SEQ ID NO: 523 - RNA, 21 nt, Homo sapiens
uaacagucuccagucacggcc

SEQ ID NO: 524 - RNA, 22 nt, Homo sapiens
uagcuuaucagacugauguuga

SEQ ID NO: 525 - RNA, 23 nt, Homo sapiens
agcuacauugucugcuggguuuc

SEQ ID NO: 526 - RNA, 21 nt, Homo sapiens
agcuacaucuggcuacugggu

SEQ ID NO: 527 - RNA, 22 nt, Homo sapiens
ugucaguuugucaaauacccca

SEQ ID NO: 528 - RNA, 22 nt, Homo sapiens
uucaaguaauccaggauaggcu

SEQ ID NO: 529 - RNA, 21 nt, Homo sapiens
uucacaguggcuaaguuccgc

SEQ ID NO: 530 - RNA, 22 nt, Homo sapiens
cacuagauugugagcuccugga

SEQ ID NO: 531 - RNA, 22 nt, Homo sapiens
uagcaccaucugaaaucgguua

SEQ ID NO: 532 - RNA, 23 nt, Homo sapiens
uaagugcuuccauguuuugguga

SEQ ID NO: 533 - RNA, 23 nt, Homo sapiens
uaagugcuuccauguuuuaguag

SEQ ID NO: 534 - RNA, 22 nt, Homo sapiens
uguaaacauccuacacucagcu

SEQ ID NO: 535 - RNA, 23 nt, Homo sapiens
uguaaacauccuacacucucagc

SEQ ID NO: 536 - RNA, 21 nt, Homo sapiens
aggcaagaugcuggcauagcu

SEQ ID NO: 537 - RNA, 22 nt, Homo sapiens
aaaagcuggguugagagggcga

SEQ ID NO: 538 - RNA, 21 nt, Homo sapiens
cacauuacacggucgaccucu

SEQ ID NO: 539 - RNA, 22 nt, Homo sapiens
cuggcccucucugcccuuccgu

SEQ ID NO: 540 - RNA, 23 nt, Homo sapiens
ucucacacagaaaucgcacccgu

SEQ ID NO: 541 - RNA, 22 nt, Homo sapiens
uaaugccccuaaaaauccuuau

SEQ ID NO: 542 - RNA, 22 nt, Homo sapiens
uuauaauacaaccugauaagug

SEQ ID NO: 543 - RNA, 21 nt, Homo sapiens
aucauagaggaaaauccacgu

SEQ ID NO: 544 - RNA, 21 nt, Homo sapiens
aacauagaggaaauuccacgu

SEQ ID NO: 545 - RNA, 23 nt, Homo sapiens
uagugcaauauugcuuauagggu

SEQ ID NO: 546 - RNA, 22 nt, Homo sapiens
aagacgggaggaaagaagggag

SEQ ID NO: 547 - RNA, 22 nt, Homo sapiens
aguggggaacccuuccaugagg

SEQ ID NO: 548 - RNA, 21 nt, Homo sapiens
caaagcgcuucccuuuggagc

SEQ ID NO: 549 - RNA, 21 nt, Homo sapiens
gaaagcgcuucucuuuagagg

SEQ ID NO: 550 - RNA, 23 nt, Homo sapiens
gaacgcgcuucccuauagagggu

SEQ ID NO: 551 - RNA, 22 nt, Homo sapiens
caugccuugaguguaggaccgu

SEQ ID NO: 552 - RNA, 22 nt, Homo sapiens
cacgcucaugcacacacccaca

SEQ ID NO: 553 - RNA, 23 nt, Homo sapiens
aaacucuacuuguccuucugagu

SEQ ID NO: 554 - RNA, 23 nt, Homo sapiens
ugugcuugcucgucccgcccgca

SEQ ID NO: 555 - RNA, 23 nt, Homo sapiens
caaagugcuguucgugcagguag

SEQ ID NO: 556 - RNA, 22 nt, Homo sapiens
cacccguagaaccgaccuugcg

SEQ ID NO: 557 - DNA, 57 bp, Homo sapiens
gatgaccccaggtaactctgagtgtgtcgctgatgccatcaccgcagcgctctgacc

SEQ ID NO: 558 - RNA, 22 nt, Homo sapiens
uuuucaacucuaaugggagaga

SEQ ID NO: 559 - RNA, 20 nt, Homo sapiens
ucacuguucagacaggcgga

SEQ ID NO: 560 - RNA, 22 nt, Homo sapiens
cggaugagcaaagaaagugguu

SEQ ID NO: 561 - RNA, 22 nt, Homo sapiens
cggaugagcaaagaaagugguu

SEQ ID NO: 562 - RNA, 22 nt, Homo sapiens
augggugaauuuguagaaggau

SEQ ID NO: 563 - RNA, 18 nt, Homo sapiens
gucccuguucaggcgcca

SEQ ID NO: 564 - RNA, 17 nt, Homo sapiens
ucccuguucgggcgcca

SEQ ID NO: 565 - RNA, 22 nt, Homo sapiens
uucauucggcuguccagaugua

SEQ ID NO: 566 - RNA, 23 nt, Homo sapiens
uauggcuuuucauuccuauguga

SEQ ID NO: 567 - RNA, 20 nt, Homo sapiens
uacaguauagaugauguacu

SEQ ID NO: 568 - RNA, 21 nt, Homo sapiens
cuagacugaagcuccuugagg

SEQ ID NO: 569 - RNA, 21 nt, Homo sapiens
ugauauguuugauauuggguu

SEQ ID NO: 570 - RNA, 23 nt, Homo sapiens
ugugcaaauccaugcaaaacuga

SEQ ID NO: 571 - RNA, 22 nt, Homo sapiens
uagcuuaucagacugauguuga

SEQ ID NO: 572 - RNA, 22 nt, Homo sapiens
cuuucagucggauguuuacagc

SEQ ID NO: 573 - RNA, 23 nt, Homo sapiens
ucaagagcaauaacgaaaaaugu

SEQ ID NO: 574 - RNA, 22 nt, Homo sapiens
uggcagugucuuagcugguugu

SEQ ID NO: 575 - RNA, 21 nt, Homo sapiens
acuggacuuggagucagaagg

SEQ ID NO: 576 - RNA, 22 nt, Homo sapiens
aaagugcuuccuuuuagagggu

SEQ ID NO: 577 - RNA, 21 nt, Homo sapiens
ugaguuggccaucugagugag

SEQ ID NO: 578 - RNA, 22 nt, Homo sapiens
uggucuaggauuguuggaggag

SEQ ID NO: 579 - RNA, 21 nt, Homo sapiens
agggggaaaguucuauagucc

SEQ ID NO: 580 - RNA, 23 nt, Homo sapiens
aucgcugcgguugcgagcgcugu

SEQ ID NO: 581 - RNA, 22 nt, Homo sapiens
acuuguaugcuagcucagguag

SEQ ID NO: 582 - RNA, 17 nt, Homo sapiens
ucucgcuggggccucca

SEQ ID NO: 583 - RNA, 23 nt, Homo sapiens
ucugcucauaccccaugguuucu

SEQ ID NO: 584 - RNA, 22 nt, Homo sapiens
uauaccucaguuuuaucaggug

SEQ ID NO: 585 - RNA, 22 nt, Homo sapiens
cacuggcuccuuucuggguaga

SEQ ID NO: 586 - RNA, 23 nt, Homo sapiens
caaagugcuguucgugcagguag

SEQ ID NO: 587 - DNA, 56 bp, Homo sapiens
gatgaccccaggtaactctgagtgtgtcgcgatgccatcaccgcagcgctctgacc

SEQ ID NO: 588 - RNA, 22 nt, Rattus norvegicus
uucccuuugucauccuaugccu

SEQ ID NO: 589 - RNA, 22 nt, Rattus norvegicus
cugugcgugugacagcggcuga

SEQ ID NO: 590 - RNA, 21 nt, Rattus norvegicus
agcuacaucuggcuacugggu

SEQ ID NO: 591 - RNA, 21 nt, Rattus norvegicus
aucacauugccagggauuacc

SEQ ID NO: 592 - RNA, 22 nt, Rattus norvegicus
uagcaccauuugaaaucgguua

SEQ ID NO: 593 - RNA, 22 nt, Rattus norvegicus
aaaagcuggguugagagggcga

SEQ ID NO: 594 - RNA, 22 nt, Rattus norvegicus
uuuuucauuauugcuccugacc

SEQ ID NO: 595 - RNA, 22 nt, Rattus norvegicus
uuuugcgauguguuccuaauau

SEQ ID NO: 596 - RNA, 22 nt, Rattus norvegicus
ugaaacauacacgggaaaccuc

SEQ ID NO: 597 - RNA, 22 nt, Rattus norvegicus
ugugacagauugauaacugaaa

SEQ ID NO: 598 - RNA, 21 nt, Rattus norvegicus
aauggcgccacuaggguugug

SEQ ID NO: 599 - RNA, 22 nt, Rattus norvegicus
ugcggggcuagggcuaacagca

SEQ ID NO: 600 - RNA, 22 nt, Rattus norvegicus
uagguaguuucguguuguuggg

SEQ ID NO: 601 - RNA, 22 nt, Rattus norvegicus
uauagggauuggagccguggcg

SEQ ID NO: 602 - RNA, 22 nt, Rattus norvegicus
acugcccuaagugcuccuucug

SEQ ID NO: 603 - RNA, 22 nt, Rattus norvegicus
uggaauguaaggaagugugugg

SEQ ID NO: 604 - RNA, 22 nt, Rattus norvegicus
ugccugucuacacuugcugugc

SEQ ID NO: 605 - RNA, 22 nt, Rattus norvegicus
ccuguucuccauuacuuggcuc

SEQ ID NO: 606 - RNA, 22 nt, Rattus norvegicus
gcugguuucauauggugguuua

SEQ ID NO: 607 - RNA, 22 nt, Rattus norvegicus
cuuucagucggauguuuacagc

SEQ ID NO: 608 - RNA, 21 nt, Rattus norvegicus
ccucugggcccuuccuccagu

SEQ ID NO: 609 - RNA, 21 nt, Rattus norvegicus
acuggacuuggagucagaagg

SEQ ID NO: 610 - RNA, 21 nt, Rattus norvegicus
uauacauacacgcacacauag

SEQ ID NO: 611 - RNA, 22 nt, Rattus norvegicus
auauacauacacacaccaacac

SEQ ID NO: 612 - RNA, 22 nt, Rattus norvegicus
cacagcucccaucucagaacaa

SEQ ID NO: 613 - RNA, 18 nt, Rattus norvegicus
aucucgcuggggccucca

SEQ ID NO: 614 - RNA, 21 nt, Rattus norvegicus
cagugcaauuaaaagggggaa

SEQ ID NO: 615 - RNA, 24 nt, Rattus norvegicus
gauugcugugcgugcggaaucgac

SEQ ID NO: 616 - RNA, 20 nt, Rattus norvegicus
guagaggagauggcgcaggg

SEQ ID NO: 617 - RNA, 22 nt, Rattus norvegicus
auaaagcuagauaaccgaaagu